(19) United States
(12) Patent Application Publication
(10) Pub. No.: US 2024/0384272 A1
(43) Pub. Date: Nov. 21, 2024

(54) ANGPTL4 OLIGONUCLEOTIDES INFLUENCING THE REGULATION OF THE FATTY ACID METABOLISM

(71) Applicant: Lipigon Pharmaceuticals AB, UMEÅ (SE)

(72) Inventors: Frank JASCHINSKI, Marburg (DE); Anne SADEWASSER, Marburg (DE); Sven MICHEL, Marburg (DE)

(21) Appl. No.: 18/671,009

(22) Filed: May 22, 2024

Related U.S. Application Data
(62) Division of application No. 17/293,356, filed on May 12, 2021, now Pat. No. 12,024,708.

(30) Foreign Application Priority Data
Nov. 13, 2018 (EP) 18206087.1

(51) Int. Cl. C12N 15/113

(52) U.S. Cl. CPC C12N 15/113; C12N 2310/11; C12N 2310/315; C12N 2310/3231

(57) ABSTRACT

An ANGPTL4 inhibitor consisting of an oligonucleotide has 12 to 22 nucleotides. At least one of the nucleotides is modified, and the oligonucleotide hybridizes with a nucleic acid sequence of human and/or mouse ANGPTL4 and inhibits the expression of ANGPTL4. Also disclosed herein is a pharmaceutical composition of the ANGPTL4 inhibitor and a pharmaceutically acceptable carrier, excipient, diluent, or a combination thereof.

### PRIORITY AND CROSS REFERENCE TO RELATED APPLICATIONS

This application is a Divisional Application of U.S. patent application Ser. No. 17/293,356, filed May 12, 2021, which is the U.S. National Stage Application under 35 U.S.C. § 371 of International Application No. PCT/EP2019/081161, filed Nov. 13, 2019, designating the U.S. and published in the English language as WO 2020/099478 A2 on May 22, 2020, which claims the benefit of European Patent Application No. EP 18206087.1, filed Nov. 13, 2018. Any and all applications for which a foreign or a domestic priority is claimed is/are identified in the Application Data Sheet filed herewith and is/are hereby incorporated by reference in their entirety under 37 C.F.R. § 1.57.

### SEQUENCE LISTING IN ELECTRONIC FORMAT

The present application is being filed along with an Electronic Sequence Listing in ST.26 format via Patent Center. The Electronic Sequence Listing is provided as a file entitled ZACC231001D2SEQLIST.xml, created and last saved on May 21, 2024, which is 347,135 bytes in size. The information in the Electronic Sequence Listing is incorporated herein by reference in its entirety.

### FIELD

The present invention refers to an inhibitor of ANGPTL4 such as an antisense oligonucleotide and a pharmaceutical composition comprising such an inhibitor as well as its use for treating a cardiovascular disease, obesity, diabetes type II, homozygote familial hypercholesterolemia (HoFH), heterozygote familial hypercholesterolemia (HeFH) or dyslipidemia.

### TECHNICAL BACKGROUND

Disturbed plasma lipids are well-known risk factors in cardiometabolic disease. Successful treatment for elevated LDL-cholesterol has been given since the mid-80's and the following decades focus has broadened towards other lipid classes such as HDL-cholesterol and triglycerides. Epidemiological studies have revealed that increased plasma triacylglycerol (TG) and concomitant remnant cholesterol is an independent risk factor for coronary heart disease (Cullen, 2000). Furthermore, hypertriglyceridemia (HTG) is a hallmark of the metabolic syndrome (MS) and is often accompanied by obesity and insulin resistance (Reaven, 1995). The increased risk of type 2 diabetes and cardiovascular disease (CVD) associated with metabolic syndrome and HTG suggests that maintenance of plasma TG homeostasis is highly desirable.

Patients with severe hypertriglyceridemia can also develop pancreatitis (Athyros, 2002), particularly when TG levels exceed 1000-1500 mg/dl (Tsuang, 2009). As many as 40 different genes are now known to regulate plasma TG (Johansen, 2011), but only a few monogenetic disorders are known to markedly increase TG (Nordestgaard and Varbo, 2014). These comprises FCS which is described in more detail below.

Lipolysis is a key step in clearance of TG-rich lipoproteins that takes place on the luminal surface of capillaries of heart, skeletal muscle, and adipose tissues. LPL synthesized in muscle and adipocytes is translocated to capillary endothelial cells. Rare genetic defects in lipoprotein lipase (LPL) (Benlian, 1996), the main enzyme responsible for the hydrolysis of TG on lipoproteins, can lead to familial chylomicronemia syndrome (FCS) characterized by plasma TG levels well over >10 mmol. Homozygous defects in apolipoprotein C-II (apoC-II), a key protein activator of LPL, can also lead to a similar hypertriglyceridemic phenotype (Breckenridge, 1978). More recently, defects in GPIHBP1, a protein that links LPL to the surface of endothelial cells (Beigneux, 2007), and mutations in apolipoprotein A-V (ApoA-V) (Ishihara, 2005) have also been described to cause hypertriglyceridemia in humans. Genetic defects in the lipase specific chaperone LMF1 has also been found to promote FCS. Taken together about 2-3:1000000 patients have FCS.

Not only LPL activating factors affect the LPL system; loss of function mutations in LPL negative regulators such as apoC3 and ANGPTL4, ANGPTL3 or ANGPTL8 has been shown to promote a favorable plasma lipid profile and a reduced risk for metabolic diseases. ANGPTL4 is a regulator of different lipases and LPL in particular. The protein is unfolding chaperones that break up the dimeric catalytically active form of LPL into inactive monomers which is an irreversible event. The ANGPTLs are the only known factors to regulate LPL in this manner, compared to e.g. apoC3 which displaces LPL from lipid substrates. In addition ANGPTL4 affects hepatic lipase and endothelial lipase thus affecting not only the TG moiety of plasma lipids but also LDL-c and HDL-c. ANGPTL4 is a fasting induced factor expressed also by the liver but to a relevant extent by adipose tissue and skeletal muscle as well, i.e., ANGPTL4 is expressed ubiquitary. The expression of ANGPTL4 is regulated by different stimuli; it is for example induced in the liver by Peroxisome Proliferator-Activator Receptor (PPAR)α, PPARδ and the glucocorticoid receptor (GR), respectively. Animal models deficient for these ANGPTLs show increased LPL activity and decreased plasma lipids and mice with transgenic overexpression for the human variants show the opposite. The findings from animal studies are supported by human deficiency and loss of function mutations which correlates for ANGPTL4 with plasma TG levels and HDL-c. The ANGPTL4 gene shows a link to cardiometabolic diseases.

Thus, information to date provides new insights into the coordinate activities of LPL, GPIHBP1, ANGPTLs and apoA-V in plasma TG homeostasis. Among these factors, ANGPTL4 also regulates plasma cholesterol levels i.e. LDL-c, HDL-c and remnant-c intriguingly without being all dependent on the LDL-receptor which in most cases is non-functional in homozygote familial hypercholesterolemia (HoFH) and heterozygote familial hypercholesterolemia (HeFH). This provides an opportunity for an “all-purpose” plasma lipid drug while targeting ANGPTL4.

ANGPTL4 regulates the activity of the lipoprotein lipase that plays an important role in the intake of free fatty acids into the liver. Dysregulation of lipoprotein lipase can lead to a lipid excess in the cells, which results for example in obesity, diabetes type II or cardiovascular diseases.

ANGPTL4 knock-out mice show reduced triglyceride (TG) level based on increased degradation of very low-density lipoprotein (VLDL) and reduced VLDL production. The cholesterol level is influenced moderately. Food having high lipid level results in ANGPTL4 knock-out mice which are treated with monoclonal antibodies to reduced viability due to lesions of lipogranuloma of the intestinal tissue, the effluent lymphatic system and/or the mesenteric lymph nodes (Desai et al., 2007 PNAS). Humans being heterozygous for the ANGPTL4 variant E40K show significant lower plasma TG level when fasting. Also the high density lipoprotein (HDL) cholesterol levels were significantly higher in E40K heterozygous humans. As the combination of high TG and low HDL cholesterol level leads to an increased risk to suffer from cardiovascular diseases, the reduction or inhibition of ANGPTL4 may reduce the risk. ANGPTL4 null alleles exist in humans, but a pathology comparable to ANGPTL4 knock-out mice has not been identified so far.

Oligonucleotides of the present invention inhibiting the expression of ANGPTL4 reduce for example the plasma lipid level independent of LDL receptor functionality, which is relevant for example for use of the oligonucleotides in treating homozygote familial hypercholesterolemia (HoFH) or heterozygote familial hypercholesterolemia (HeFH), where the LDL receptor is defect.

ANGPTL4 is not only involved in the regulation of the fatty acid metabolism, but it is also involved in influenza infection. ANGPTL4 is for example upregulated by STAT3-mediated mechanism during influenza pneumonia and is a potential biomarker for respiratory infection and pneumonia (Li et al., Cell Reports 10, Feb. 2015).

So far no antisense oligonucleotide exists which is highly efficient in reduction and inhibition, respectively, of ANGPTL4 expression and hybridizes with ANGPTL4 mRNA and/or pre-mRNA. Studies with siRNA to inhibit ANGPTL4 expression showed that in vivo inhibition is only possible if siRNA is packed in suitable packaging material. Even if siRNA is packed the efficiency on the inhibition of mRNA expression can often not be improved.

An oligonucleotide of the present invention is very successful in the inhibition of the expression of ANGPTL4. The mode of action of an oligonucleotide differs from the mode of action of an antibody or small molecule, and oligonucleotides are highly advantageous regarding for example
(i) the penetration into tissues,
(ii) the blocking of multiple functions and activities, respectively, of a target,
(iii) the combination of oligonucleotides with each other or an antibody or a small molecule, and
(iv) the inhibition of intracellular effects which are not accessible or not specifically accessible for an antibody or inhibitable via a small molecule.

### SUMMARY

The present invention refers to an ANGPTL4 inhibitor consisting of an oligonucleotide comprising or consisting of for example 12 to 22 nucleotides, 15 to 20 nucleotides, or 15, 16, 17, 18, 19 or 20 nucleotides, wherein at least one of the nucleotides is modified. The ANGPTL4 oligonucleotide hybridizes for example with a nucleic acid sequence of ANGPTL4 of SEQ ID NO.1 (human; NM_139314), ANGPTL4 of SEQ ID NO. 2 (human; GRCh38_19_8364151_8374373) ANGPTL4 of SEQ ID NO.58 (mouse; NM_020581.2) and/or ANGPTL4 of SEQ ID NO.59 (mouse; GRCm38: 17:33773750:33781575), wherein the oligonucleotide inhibits the expression of ANGPTL4. The modified nucleotide is for example selected from the group consisting of a bridged nucleic acid such as LNA, cET, ENA, 2′Fluoro modified nucleotide, 2′O-Methyl modified nucleotide, 2′ O-Methoxyethyl modified nucleotide and a combination thereof.

The ANGPTL4 oligonucleotide of the present invention hybridizes for example with an active area selected from position 1732-1759 (e.g., A24044He, SEQ ID NO.47; A24076He, SEQ ID NO.47) and/or from position 234-261 (e.g. A24102He, SEQ ID NO. 177; A24103He, SEQ ID NO. 178) and/or from position 1264-1293 (e.g. A24110He, SEQ ID NO. 186; A24111He, SEQ ID NO. 187) of SEQ ID NO. 1 and/or from position 2800-2872 (eg. A24083Hi, SEQ ID NO. 158; A24085Hi, SEQ ID NO. 160; A24086Hi, SEQ ID NO. 161; A24087Hi, SEQ ID NO. 162) and/or from position 3415-3442 (e.g. A24089Hi, SEQ ID NO. 164) and/or from position 4968-4994 (e.g. A24097Hi, SEQ ID NO. 172) of SEQ ID NO.2 or a combination thereof. It inhibits the expression of ANGPTL4 for example at a nanomolar or micromolar concentration.

The present invention is further directed to a pharmaceutical composition comprising an ANGPTL4 inhibitor of the present invention and a pharmaceutically acceptable carrier, excipient, dilutant or a combination thereof. The inhibitor and the pharmaceutical composition, respectively, are for use in a method of preventing and/or treating a disorder, where an ANGPTL4 imbalance is involved. Such disorder is for example a cardiometabolic disease, obesity, diabetes such as type 2 diabetes mellitus, hypercholesterolemia, hypertriglyceridemia (HTG), dyslipidemia, pancreatitis, metabolic syndrome, familial chylomicronemia syndrome (FCS), influenza infection and/or cancer. Hypercholesterolemia is for example homozygote familial hypercholesterolemia (HoFH) and heterozygote familial hypercholesterolemia (HeFH), cancer is for example breast cancer, lung cancer, malignant melanoma, lymphoma, skin cancer, bone cancer, prostate cancer, liver cancer, brain cancer, cancer of the larynx, gall bladder, pancreas, testicular, rectum, parathyroid, thyroid, adrenal, neural tissue, head and neck, colon, stomach, bronchi, kidneys, basal cell carcinoma, squamous cell carcinoma, metastatic skin carcinoma, osteo sarcoma, Ewing's sarcoma, reticulum cell sarcoma, liposarcoma, myeloma, giant cell tumor, small-cell lung tumor, islet cell tumor, primary brain tumor, meningioma, acute and chronic lymphocytic and granulocytic tumors, acute and chronic myeloid leukemia, hairy-cell tumor, adenoma, hyperplasia, medullary carcinoma, intestinal ganglioneuromas, Wilm's tumor, seminoma, ovarian tumor, leiomyomata tumor, cervical dysplasia, retinoblastoma, soft tissue sarcoma, malignant carcinoid, topical skin lesion, rhabdomyosarcoma, Kaposi's sarcoma, osteogenic sarcoma, malignant hypercalcemia, renal cell tumor, polycythemia vera, adenocarcinoma, anaplastic astrocytoma, glioblastoma multiforme, leukemia, or epidermoid carcinoma.

The ANGPTL4 inhibitor or the pharmaceutical composition of the present invention comprising the ANGPTL4 inhibitor are administered locally or systemically.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### BRIEF DESCRIPTION OF THE DRAWINGS

FIG. 1 shows an initial screen of 42 human ANGPTL4-specific antisense oligonucleotides. A control oligonucleotide that does not have sequence complementarity to any human or mouse mRNA (Neg1; SEQ ID NO.57) was included as a control. Human epithelioid cervix carcinoma cells (HeLa) were treated with the respective oligonucleotides at a single concentration of 10 μM for three days. Three days after start of treatment, cells were lysed and ANGPTL4 and HPRT1 mRNA levels were determined using the QuantiGene Singleplex RNA assay. HPRT1 was used as a housekeeping gene for normalization of ANGPTL4 expression. Residual ANGPTL4-mRNA expression relative to mock-treated cells (“no oligo” set as 1). Triplicate wells, mean+/−SD (FIG. 1).

FIG. 2 depicts another screen of human ANGPTL4-specific antisense oligonucleotides in SK-OV3 cells to investigate the residual ANGPTL4 mRNA expression.

FIG. 3 depicts further human ANGPTL4-specific antisense oligonucleotides tested in HeLa cells at a concentration of 10 μM. Four of the tested antisense oligonucleotides show more than 50% knockdown of ANGPTL4 mRNA (equivalent to residual mRNA level of <0.5).

FIG. 4 shows another screen of further ANGPTL4-specific antisense oligonucleotides in SK-OV3 cells to investigate the residual ANGPTL4 mRNA expression.

FIG. 5 depicts HEK-Blue™ hTLR9 cells used to study the stimulation of human TLR9 by monitoring the activation of nuclear factor ‘kappa-light-chain-enhancer’ of activated B-cells (NF-KB)-dependent secreted embryonic alkaline phosphatase (SEAP)-production in vitro. ANGPTL4 oligonucleotides A24022Hi (SEQ ID NO.25), A24023Hi (SEQ ID NO.26), A24071Hi (SEQ ID NO.54) and A24076He (SEQ ID NO.47) as well as controls Neg1 (SEQ ID NO.57) and ODN2006 (5′-TCGTCGTTTTGTCGTTTTGTCGTT-3′ PTO-modified-Invivogen cat. no. tlrl-2006; SEQ ID NO.153) were analyzed.

FIG. 6 shows the human ANGPTL4-specific antisense oligonucleotides A24022Hi (SEQ ID NO. 25), A24023Hi (SEQ ID NO. 26), A24071Hi (SEQ ID NO.54) and A24076He (SEQ ID NO.47) with most potent knockdown efficacy in HeLa and SK-OV3 cells, which were selected for determination of half maximal inhibitory concentration (IC₅₀) values. Primary hepatocytes were treated with the respective ANGPTL4 antisense oligonucleotides at different concentrations (5000 nM, 1000 nM, 200 nM, 40 nM, 8 nM, 1.6 nM) for three days and simultaneously treated with PPARγ (1 M). Three days after start of treatment, mRNA expression was analyzed using the QuantiGene Singleplex RNA assay as depicted in FIG. 6. For graphic representation mock-treated cells were set as 0.32 nM.

FIG. 7 shows a first single dose efficacy screen of the additional ANGPTL4-specific antisense oligonucleotides (ASOs) in primary hepatocytes. 25,000 cells/well were seeded in 96-well collagen I-coated flat bottom plates and treated with the respective ASOs at a final concentration of 5 μM. To induce ANGPTL4 mRNA expression, cells were simultaneously treated with 1 μM PPARγ. As a vehicle control, cells were treated with equal amount of DMSO (“DMSO”). Every 24 h, 70 μl of supernatant was replaced with fresh medium containing PPARγ as well as the respective ASOs at 5 μM or DMSO. After three days, cells were lysed and human HPRT1 as well as human ANGPTL4 mRNA expression was measured using the QuantiGene RNA Singleplex assay. ANGPTL4-mRNA expression values were normalized to expression of the housekeeping gene HPRT1. Residual ANGPTL4-mRNA expression relative to mock-treated cells (“no oligo” set as 1 (n=42), SD=0.24) is shown. Solid line and dotted lines indicate 70% and 0% knockdown efficacy (upper graph) or 100% HPRT1 expression of mock-treated cells (“no oligo” set as 100 (n=42), SD=35.8, graph below). respectively. Data are represented as mean of triplicate wells+/−SD. Positive control ASOs are marked by an asterix.

FIG. 8 depicts a second single dose efficacy screen of the additional ANGPTL4-specific ASOs in primary hepatocytes. 25,000 cells/well were seeded in 96-well collagen I-coated flat bottom plates and treated with the respective ASOs at a final concentration of 5 μM. To induce ANGPTL4 mRNA expression, cells were simultaneously treated with 1 μM PPARγ. As a vehicle control, cells were treated with equal amount of DMSO (“DMSO”) or left untreated (“-DMSO”). Every 24 h, 70 μl of supernatant was replaced with fresh medium containing PPARγ as well as the respective ASOs at 5 μM or DMSO or medium only. After three days, cells were lysed and human HPRT1 as well as human ANGPTL4 mRNA expression were measured using the QuantiGene RNA Singleplex assay. ANGPTL4-mRNA expression values were normalized to expression of the housekeeping gene HPRT1. Residual ANGPTL4-mRNA expression relative to mock-treated cells (“no oligo” set as 1 (n=36), SD=0.24) is shown. Solid line and dotted lines indicate 70% and 0% knockdown efficacy (upper graph) or 100% HPRT1 expression of mock-treated cells (“no oligo” set as 100 (n=36), SD=19.3. graph below), respectively. Data are represented as mean of sextuplicate (A24076He and negative control oligonucleotides), nonuplicate (-DMSO) or triplicate (all other ASOs) wells+/−SD. Positive control ASO is marked by an asterix.

FIGS. 9A-9D depict NF-KB-activation in HEK-Blue hTLR9 SEAP reporter cells. HEK-Blue-hTLR9 cells were seeded in flat-bottom 96-well plates and treated with the indicated oligonucleotides for 24 h. Cell supernatants were harvested after the incubation and incubated for 4 h (FIGS. 9A, 9C, and 9D) or 3.5 h (FIG. 9B) with QUANTI-Blue solution. SEAP activity was determined by measurement of the optical density (OD) at 620 nm. Data for ANGPTL4-specific ASOs and neg1 treated cells are depicted as mean of triplicates+/−SD of OD units relative to OD units from cells stimulated with 5000 nM ODN2006 (set to 100%). Data for ODN2006 treated cells are depicted as mean+/−SD of sextuplicates (A), as mean+/−SD of triplicates (D) or as the mean+/−SD of the means of triplicates on each plate (B-C).

FIG. 10 shows IC₅₀ determination of selected ANGPTL4 ASOs. 25,000 primary human hepatocytes/well were seeded in 96-well plates and treated with different concentrations (5000 nM, 1000 nM, 200 nM, 40 nM, 8 nM, 1.6 nM) of the respective ASO. Every 24 h, 70 μl of supernatant was replaced with fresh medium containing the respective ASOs at indicated concentrations. After three days, cells were lysed and HPRT1 and ANGPTL4 mRNA expression was measured using the QuantiGene RNA Singleplex assay. ANGPTL4-mRNA expression values were normalized to expression of the housekeeping gene HPRT1. Residual ANGPTL4-mRNA expression relative to mock-treated cells (set as 1 (n=36), SD=0.26). Data are represented as mean of triplicate wells+/−SD.

FIG. 11 shows the efficacy of selected ANGPTL4 ASOs on target gene expression after transfection of cynomolgus hepatocytes. 25,000 primary cynomolgus hepatocytes/well were seeded in 96-well plates and transfected with different concentrations (2 nM, 0.2 nM) of the respective ASO. After 24 h incubation at 37° C., cells were lysed and HPRT1 and ANGPTL4 mRNA expression were measured using the QuantiGene RNA Singleplex assay. ANGPTL4-mRNA expression values were normalized to expression of the housekeeping gene HPRT1. Residual ANGPTL4-mRNA expression relative to mock-treated cells (set as 1 (n=12), SD=0.25) is depicted. Data are represented as mean of triplicate wells+/−SD.

FIG. 12 depicts mouse embryonic fibroblasts (3T3 cells) separately treated with the mouse ANGPTL4-specific antisense oligonucleotides as shown in FIG. 12 at a single concentration of 10 μM. Three days after start of treatment, the mRNA levels were determined by QuantiGene RNA Singleplex assay. Hprt1 was used as a housekeeping gene for normalization of ANGPTL4 expression. Eight ANGPTL4 antisense oligonucleotides, which are A24047M (SEQ ID NO.106), A24020M (SEQ ID NO.79), A24017M (SEQ ID NO.76), A24021M (SEQ ID NO.80), A24049M (SEQ ID NO.108), A24018M (SEQ ID NO.77), A24041M (SEQ ID NO.100) and A24010M (SEQ ID NO.69), reduced the normalized ANGPTL4 expression by more than 50%.

FIG. 13 depicts mouse renal carcinoma Renca cells separately treated with the ANGPTL4-specific antisense oligonucleotides as shown in FIG. 13 at a single concentration of 10 μM. After three days the mRNA levels were determined by QuantiGene RNA Singleplex assay. Hprt1 was used as a housekeeping gene for normalization of ANGPTL4 expression. A24020M (SEQ ID NO.79) and A24019M (SEQ ID NO.78) resulted in ANGPTL4 knockdown of more than 50% (equivalent to residual mRNA level of <0.5).

FIG. 14 depicts 9 already tested as well as 24 further mouse ANGPTL4-specific antisense oligonucleotides tested in mouse breast cancer cells 4T1. Cells were treated with 5 μM of respective ANGPTL4 antisense oligonucleotides as shown in FIG. 14 without using a transfection reagent. After three days, cell supernatant was replaced by fresh medium containing 5 μM of the respective ANGPTL4 antisense oligonucleotides and incubated for further three days. Afterwards, the mRNA levels were determined by QuantiGene RNA Singleplex assay. Gapdh was used as a housekeeping gene for normalization of ANGPTL4 expression. 12 of the tested antisense oligonucleotides, which are A24017M (SEQ ID NO.76), A24070M (SEQ ID NO.127), A24020M (SEQ ID NO.79), A24019M (SEQ ID NO.78), A24069M (SEQ ID NO.126), A24021M (SEQ ID NO.80), A24011M (SEQ ID NO.70), A24073M (SEQ ID NO. 130), A24018M (SEQ ID NO.77), A24055M (SEQ ID NO.114), A24010M (SEQ ID NO.69), and A24065M (SEQ ID NO.79) show more than 80% knockdown of ANGPTL4 mRNA (equivalent to residual mRNA level of <0.2).

FIG. 15 shows 21 mouse ANGPTL4-specific antisense oligonucleotides tested in 4T1 cells by treating them with 5 μM of the respective ANGPTL4 antisense oligonucleotide as shown in FIG. 15. After three days, cell supernatant was replaced by fresh medium containing 5 μM of the respective ANGPTL4 antisense oligonucleotide and incubated for additional three days. Afterwards, the mRNA levels were determined by QuantiGene RNA Singleplex assay. Hprt1 was used as a housekeeping gene for normalization of ANGPTL4 expression. A24047M (SEQ ID NO.106), A24095Mi (SEQ ID NO. 151), A24093Mi (SEQ ID NO. 149), A24020M (SEQ ID NO.79), A24090Mi (SEQ ID NO. 146), and A24082Mi (SEQ ID NO. 139) show more than 50% knockdown of ANGPTL4 mRNA (equivalent to residual mRNA level of <0.5).

FIG. 16 depicts A24018M (SEQ ID NO.77), A24019M (SEQ ID NO.78), A24020M (SEQ ID NO.79), A24021M (SEQ ID NO.80), A24047M (SEQ ID NO.106), A24054M (SEQ ID NO.113), A24065M (SEQ ID NO.79), A24070M (SEQ ID NO.127), A24072M (SEQ ID NO.129), A24082M (SEQ ID NO.139) and A24095Mi (SEQ ID NO.151) with most potent knockdown efficacy in 3T3, Renca and 4T1 cells, which were selected for determination of half maximal inhibitory concentration (IC₅₀) values. 4T1 cells were treated with these ANGPTL4 antisense oligonucleotides at different concentrations (5000 nM, 1000 nM, 200 nM, 40 nM, 8 nM, 1.6 nM) for three days. After three days, cell supernatant was replaced by fresh medium containing 5 μM of the respective ANGPTL4 antisense oligonucleotides and incubated for additional three days. Then, mRNA expression was analyzed using the QuantiGene Singleplex RNA assay.

FIG. 17 shows a 1^{st} single dose efficacy screen of additional mouse Angptl4-specific ASOs in 4T1 cells. 2,500 4T1 cells/well were seeded in 96-well flat bottom plates and treated with the respective ASOs at a final concentration of 5 μM. Three days after treatment, cell supernatant was replaced by fresh medium w/ASO and cells were incubated for further three days at 37° C. Afterwards, cells were lysed and mouse Hprt1 and mouse Angptl4 mRNA expression were measured using the QuantiGene RNA Singleplex assay. Angptl4-mRNA expression values were normalized to expression of the housekeeping gene Hprt1. Residual Angptl4-mRNA expression relative to mock-treated cells (“no oligo” set as 1 (n=37), SD=0.21) is shown. Solid line and dotted line indicate 75% and 0% knockdown efficacy (upper graph) or 100% Hprt1 level (graph below) (“no oligo” set as 1 (n=37), SD=0.17), respectively. Data are represented as mean of triplicate wells+/−SD.

FIG. 18 shows a 2^{nd} single-dose screen of mouse Angptl4-specific ASOs in Renca cells. 2,500 Renca cells/well were seeded in 96-well flat bottom plates and treated with the respective ASOs at a final concentration of 5 M. Three days after treatment, cell supernatant was replaced by fresh medium w/ASO and cells were incubated for further three days at 37° C. Afterwards, cells were lysed and mouse Hprt1 and mouse Angptl4 mRNA expression were measured using the QuantiGene RNA Singleplex assay. Angptl4-mRNA expression values were normalized to expression of the housekeeping gene Hprt1. Residual Angptl4-mRNA expression relative to mock-treated cells (“no oligo” set as 1 (n=37), SD=0.37) is shown. Solid line and dotted line indicate 75% and 0% knockdown efficacy (upper graph) or 100% Hprt1 level (graph below) (“no oligo” set as 1 (n=37), SD=0.36), respectively. Data are represented as mean of triplicate wells+/−SD.

FIG. 19 shows IC₅₀ determination of selected Angptl4 ASOs. 15,000 primary mouse hepatocytes/well were seeded in 96-well flat bottom plates and treated with different concentrations (5000 nM, 1000 nM, 200 nM, 40 nM, 8 nM, 1.6 nM) of the respective ASO. After three days, cells were lysed and Hprt1 and Angptl4 mRNA expression were measured using the QuantiGene RNA Singleplex assay. Angptl4-mRNA expression values were normalized to expression of the housekeeping gene Hprt1. Residual Angptl4-mRNA expression relative to mock-treated cells (set as 1 (n=36), SD=0.17). Neg1 is displayed in black. Graph below shows Hprt1 raw values relative to no oligo (set as 100 (n=36), SD=13.0). Data are represented as mean of triplicate wells+/−SD.

### DETAILED DESCRIPTION

The present invention provides a successful inhibitor of ANGPTL4 expression, which is a human or mouse oligonucleotide hybridizing with mRNA and/or pre-mRNA sequences of ANGPTL4 and inhibits the expression and activity, respectively, of ANGPTL4. mRNA comprises only exons of the ANGPTL4 encoding nucleic acid sequence, whereas pre-mRNA comprises exons and introns of the ANGPTL4 encoding nucleic acid sequence. Thus, the oligonucleotides of the present invention represent an interesting and highly efficient tool for use in a method of preventing and/or treating disorders, where the ANGPTL4 expression and activity, respectively, is increased.

In the following, the elements of the present invention will be described in more detail. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention.

An inhibitor which is an oligonucleotide of the present invention is for example an antisense oligonucleotide (ASO) consisting of or comprising 10 to 25 nucleotides, 12 to 22 nucleotides, 15 to 20 nucleotides or 16 to 18 nucleotides. The oligonucleotides for example consist of or comprise 10, 11, 12, 13, 14, 15, 16, 17, 18, 19, 20, 21, 22, 23, 24 or 25 nucleotides.

The oligonucleotide of the present invention forms for example a gapmer consisting of or comprising a central block of at least 5 nucleotides, i.e., deoxynucleotides and/or ribonucleotides, which is flanked by for example naturally and/or artificially modified nucleotides such as deoxynucleotides and/or ribonucleotides.

The oligonucleotides of the present invention comprise at least one nucleotide which is modified. The modified nucleotide is for example a bridged nucleotide such as a locked nucleic acid (LNA, e.g., 2′,4′-LNA), cET, ENA, a 2′-Fluoro modified nucleotide, a 2′O-Methyl modified nucleotide, 2′ O-Methoxyethyl modified nucleotide or a combination thereof. In some embodiments, the oligonucleotide of the present invention comprises one or more nucleotides having the same or different modifications. In addition, the oligonucleotide of the present invention optionally comprises a modified phosphate backbone, wherein the phosphate is for example a phosphorothioate.

The oligonucleotide of the present invention comprises the one or more modified nucleotide at the 3′- and/or 5′-end of the oligonucleotide and/or at any position within the oligonucleotide, wherein modified nucleotides follow in a row of for example 1, 2, 3, 4, 5, or 6 modified nucleotides, or a modified nucleotide is combined with one or more unmodified nucleotides. The following Tables 1 to 4 present examples of ANGPTL4 oligonucleotides comprising modified nucleotides for example LNA which are indicated by (+) and phosphorothioate (PTO) indicated by (*). The ANGPTL4 oligonucleotides consisting of or comprising the sequences of Table 1 or 2 (human), or Table 3 or 4 (mouse) may comprise any other modified nucleotide and/or any other combination of modified and unmodified nucleotides. ANGPTL4 oligonucleotides of Table 1 hybridize with mRNA and/or pre-m RNA of human ANGPTL4:

TABLE 1
List of antisense oligonucleotides hybridizing with
human ANGPLT4 mRNA and/or pre-mRNA for example of
SEQ ID NO. 1 and/or SEQ ID NO. 2; Neg1 is an oligonucleotide
representing a negative control which is not hybridizing with
ANGPTL4 of SEQ ID NO. 1 or SEQ ID NO. 2.
“He” means “human exonic region” and is an oligonucleotide primarily
hybridizing with mRNA of human ANGPTL4, and
“Hi” is an oligonucleotide hybridizing with introns of ANGPTL4 pre-mRNA.

| Seq | | Antisense | Antisense Sequence 5′-3′ with |
|---|---|---|---|
| ID | Name | Sequence 5′-3′ | PTO (*) and LNA (+) |
| 3 | A24001Hi | AGGCTAAGTTAGTGGCC | +A*G*G*C*T*A*A*G*T*T*A*G*T*G*G*+C*+C |
| 4 | A24002Hi | GCCAGATAGGCTAAGTT | +G*+C*C*A*G*A*T*A*G*G*C*T*A*A*+G*+T*+T |
| 5 | A24003He | CTGGAAAGAATCGGATC | +C*+T*G*+G*A*A*A*G*A*A*T*C*G*G*+A*+T*+C |
| 6 | A24004He | CGCTGGAAAGAATCGGA | +C*G*+C*T*G*G*A*A*A*G*A*A*T*+C*+G*+G*+A |
| 7 | A24005Hi | GCTAGTCTCGACAGCAG | +G*C*+T*A*G*T*C*T*C*G*A*C*A*G*C*+A*+G |
| 8 | A24006Hi | GATCCTCATGATAGGCC | +G*+A*T*C*C*T*C*A*T*G*A*T*A*G*G*+C*+C |
| 9 | A24007He | GGCCGTCGGAGCACCGC | +G*+G*C*C*G*T*C*G*G*A*G*C*A*C*C*G*+C |
| 10 | A24008Hi | CATGCTGTGGTTCGAGA | +C*+A*+T*G*C*T*G*T*G*G*T*T*C*G*+A*+G*+A |
| 11 | A24009He | CGTGCGCCAGGACATTC | +C*+G*T*G*C*G*C*C*A*G*G*A*C*A*+T*+T*+C |
| 12 | A24010He | CGTGCGCCAGGACATT | +C*+G*+T*G*C*G*C*C*A*G*G*A*C*+A*+T*+T |
| 13 | A24011He | TCCGTGCGCCAGGACAT | +T*C*C*G*T*G*C*G*C*C*A*G*G*A*+C*A*+T |
| 14 | A24012He | GGAGTCCGTGCGCCAGG | +G*G*A*G*T*C*C*G*T*G*C*G*C*C*+A*G*+G |
| 15 | A24013He | TGCGCTCCGCGTGTTCG | +T*+G*C*G*C*T*C*C*G*C*G*T*G*T*T*+C*+G |
| 16 | A24014He | TGCGCTCCGCGTGTTC | +T*+G*C*G*C*T*C*C*G*C*G*T*G*T*+T*+C |
| 17 | A24015He | GTGCGCTCCGCGTGTT | +G*+T*G*C*G*C*T*C*C*G*C*G*T*G*T*+T |
| 18 | A24016Hi | GTCGAAATGAGTCTGCA | +G*+T*+C*G*A*A*A*T*G*A*G*T*C*T*+G*+C*+A |
| 19 | A24017Hi | GCACGTCTGGAGGCTCA | +G*+C*+A*C*G*T*C*T*G*G*A*G*G*C*+T*+C*+A |
| 20 | A24018Hi | TTGAGCACGTCTGGAGG | +T*+T*+G*A*G*C*A*C*G*T*C*T*G*G*+A*+G*+G |
| 21 | A24019Hi | GGATCGCAGTGCCGCAA | +G*+G*+A*T*C*G*C*A*G*T*G*C*C*G*+C*+A*+A |
| 23 | A24020He | GCTGAATTCGCAGGTGC | +G*C*+T*G*A*A*T*T*C*G*C*A*G*G*+T*G*+C |
| 24 | A24021Hi | GATCGCAGTGCCGCAA | +G*+A*+T*C*G*C*A*G*T*G*C*C*G*+C*+A*+A |
| 25 | A24022Hi | GTGTTGTAACCTCTTGT | +G*+T*+G*T*T*G*T*A*A*C*C*T*C*T*+T*+G*+T |
| 26 | A24023Hi | CCGTGTTGTAACCTCTT | +C*+C*+G*T*G*T*T*G*T*A*A*C*C*T*+C*+T*+T |
| 27 | A24024He | CCTCATGGTCTAGGTGC | +C*+C*+T*+C*A*T*G*G*T*C*T*A*G*G*+T*+G*+C |
| 28 | A24025He | CACCTCATGGTCTAGGT | +C*A*+C*C*T*C*A*T*G*G*T*C*T*A*G*+G*+T |
| 29 | A24026He | GGCGCCTCTGAATTACT | +G*G*C*G*C*C*T*C*T*G*A*A*T*T*+A*+C*+T |
| 30 | A24027He | CGTGGCGCCTCTGAATT | +C*+G*+T*G*G*C*G*C*C*T*C*T*G*A*+A*+T*+T |
| 31 | A24028He | TCGTGGCGCCTCTGAAT | +T*C*G*T*G*G*C*G*C*C*T*C*T*G*A*+A*+T |
| 32 | A24029He | CCGCCTTGTAGGCTTCC | +C*+C*+G*C*C*T*T*G*T*A*G*G*C*T*T*C*+C |
| 33 | A24030He | ACTCGGCGTTGCCATCC | +A*+C*+T*C*G*G*C*G*T*T*G*C*C*A*T*C*+C |
| 34 | A24031He | GCCGTGTCCTCGCCACC | +G*+C*C*G*T*G*T*C*C*T*C*G*C*C*A*C*+C |
| 35 | A24032He | GAGGTCGTGATCCTGGT | +G*+A*G*G*T*C*G*T*G*A*T*C*C*T*G*+G*+T |
| 36 | A24033He | GCGGAGGTCGTGATCCT | +G*+C*+G*G*A*G*G*T*C*G*T*G*A*T*C*+C*+T |
| 37 | A24034He | CCTGCGGAGGTCGTGAT | +C*C*T*G*C*G*G*A*G*G*T*C*G*T*+G*+A*+T |
| 38 | A24035He | CTCTTGGCGCAGTTCTT | +C*+T*+C*T*T*G*G*C*G*C*A*G*T*T*+C*+T*+T |
| 39 | A24036He | GCTCTTGGCGCAGTTCT | +G*+C*+T*C*T*T*G*G*C*G*C*A*G*T*+T*+C*+T |
| 40 | A24037He | GGCTCTTGGCGCAGTTC | +G*+G*+C*T*C*T*T*G*G*C*G*C*A*G*T*+T*+C |
| 41 | A24038He | GGTGCCAAACCACCAGC | +G*+G*T*G*C*C*A*A*A*C*C*A*C*C*+A*+G*+C |
| 42 | A24039He | GGAGCGGAAGTACTGGC | +G*+G*+A*+G*C*G*G*A*A*G*T*A*C*T*G*+G*+C |
| 43 | A24040He | ATGGAGCGGAAGTACTG | +A*+T*+G*G*A*G*C*G*G*A*A*G*T*+A*+C*+T*+G |
| 44 | A24041He | GGATGGAGCGGAAGTAC | +G*+G*+A*T*G*G*A*G*C*G*G*A*A*G*+T*+A*+C |
| 45 | A24042He | GGCTGGATCAACATGGT | +G*+G*C*T*G*G*A*T*C*A*A*C*A*T*+G*+G*+T |
| 46 | A24043He | ATGTGACTGAGTCCGCC | +A*+T*+G*T*G*A*C*T*G*A*G*T*C*C*+G*+C*+C |
| 47 | A24044He | CAATGTGACTGAGTCCG | +C*+A*+A*+T*G*T*G*A*C*T*G*A*G*T*+C*+C*+G |
| 48 | A24046He | GAACTCTGTGAGCTCCG | +G*+A*+A*C*T*C*T*G*T*G*A*G*C*T*C*C*+G |
| 49 | A24063Hi | CTGGTGGACTAACACAC | +C*+T*+G*G*T*G*G*A*C*T*A*A*C*A*+C*+A*+C |
| 50 | A24064Hi | TGGACCTGACAAGGAGA | +T*+G*+G*A*C*C*T*G*A*C*A*A*G*G*+A*+G*+A |
| 51 | A24065Hi | CACACGGCATGTAAGG | +C*+A*+C*A*C*G*G*C*A*T*G*T*A*+A*+G*+G |
| 4 | A24066Hi | GCCAGATAGGCTAAGTT | +G*+C*+C*A*G*A*T*A*G*G*C*T*A*A*+G*+T*+T |
| 52 | A24067Hi | AGGCTAGTCTCGACAGC | +A*+G*+G*C*T*A*G*T*C*T*C*G*A*C*+A*+G*+C |
| 8 | A24068Hi | GATCCTCATGATAGGCC | +G*+A*+T*C*C*T*C*A*T*G*A*T*A*G*+G*+C*+C |
| 53 | A24069Hi | GATCGCAGTGCCGCAAT | +G*+A*+T*C*G*C*A*G*T*G*C*C*G*C*+A*+A*+T |
| 53 | A24070Hi | GATCGCAGTGCCGCAAT | +G*A*T*C*G*C*A*G*T*G*C*C*G*C*+A*+A*+T |
| 54 | A24071Hi | CGTGTTGTAACCTCTTG | +C*+G*+T*G*T*T*G*T*A*A*C*C*T*C*+T*+T*+G |
| 55 | A24072Hi | GATCGCAGTGCCGCA | +G*+A*+T*C*G*C*A*G*T*G*C*C*+G*+C*+A |
| 55 | A24073Hi | GATCGCAGTGCCGCA | +G*+A*+T*C*G*C*A*G*T*G*C*C*G*+C*+A |
| 56 | A24074Hi | GGATCGCAGTGCCGC | +G*+G*+A*T*C*G*C*A*G*T*G*C*+C*+G*+C |
| 5 | A24075He | CTGGAAAGAATCGGATC | +C*+T*+G*G*A*A*A*G*A*A*T*C*G*G*+A*+T*+C |
| 47 | A24076He | CAATGTGACTGAGTCCG | +C*+A*+A*T*G*T*G*A*C*T*G*A*G*T*+C*+C*+G |
| 47 | A24077He | CAATGTGACTGAGTCCG | +C*+A*+A*T*G*T*G*A*C*T*G*A*G*+T*+C*+C*+G |
| 57 | Neg1 | | +C*+G*+T*T*T*A*G*G*C*T*A*T*G*T*A*+C*+T*+T |

ANGPTL4 oligonucleotides of Table 2 also hybridize with mRNA and/or pre-m RNA of human ANGPTL4:

TABLE 2
List of antisense oligonucleotides hybridizing with
human ANGPLT4 mRNA and/or pre-mRNA for example of
SEQ ID NO. 1 and/or SEQ ID NO. 2;
Neg1, R01002, R01009, R01014 and R01019 are oligonucleotides representing
negative controls which are not hybridizing with ANGPTL4 of SEQ ID NO. 1 or
SEQ ID NO. 2.
“He” means “human exonic region” and is an oligonucleotide primarily
hybridizing with mRNA of human ANGPTL4, and
“Hi” is an oligonucleotide hybridizing with introns of ANGPTL4
pre-mRNA.
“HMe” indicates an oligonucleotide hybridizing with “human and mouse
exonic region” of ANGPTL4.

| Seq | | Antisense | Antisense Sequence 5′-3′ with |
|---|---|---|---|
| ID | Name | Sequence 5′-3′ | PTO (*) and LNA (+) |
| 154 | A24078Hi | CACCGGAGAGCATCGA | +C*+A*+C*C*G*G*A*G*A*G*C*A*T*+C*+G*+A |
| 155 | A24079Hi | CACGGCATGTAAGGAAG | +C*+A*+C*G*G*C*A*T*G*T*A*A*G*G*+A*+A*+G |
| 156 | A24080Hi | ACACACGGCATGTAAGG | +A*+C*+A*C*A*C*G*G*C*A*T*G*T*A*+A*+G*+G |
| 157 | A24081Hi | TAGTCTCGACAGCAGGT | +T*+A*+G*T*C*T*C*G*A*C*A*G*C*A*+G*+G*+T |
| 158 | A24082Hi | GGTTCGAGATGAACGGA | +G*+G*+T*T*C*G*A*G*A*T*G*A*A*C*+G*+G*+A |
| 159 | A24083Hi | TCGAGATGAACGGAGA | +T*+C*+G*A*G*A*T*G*A*A*C*G*G*+A*+G*+A |
| 160 | A24084Hi | TCTGCATCGGACACACG | +T*+C*+T*G*C*A*T*C*G*G*A*C*A*C*+A*+C*+G |
| 161 | A24085Hi | AACTTAGAGAACCGCGA | +A*+A*+C*T*T*A*G*A*G*A*A*C*C*G*+C*+G*+A |
| 162 | A24086Hi | GCCGTGAACTTAGAGAA | +G*+C*+C*G*T*G*A*A*C*T*T*A*G*A*+G*+A*+A |
| 163 | A24087Hi | TTAGAGAACCGCGAGT | +T*+T*+A*G*A*G*A*A*C*C*G*C*G*+A*+G*+T |
| 164 | A24088Hi | TGACCAGGAAGACGCTT | +T*+G*+A*C*C*A*G*G*A*A*G*A*C*G*+C*+T*+T |
| 165 | A24089Hi | TCGAAATGAGTCTGCAC | +T*+C*+G*A*A*A*T*G*A*G*T*C*T*G*+C*+A*+C |
| 166 | A24090Hi | CGTTGAGCACGTCTGGA | +C*+G*+T*T*G*A*G*C*A*C*G*T*C*T*+G*+G*+A |
| 167 | A24091Hi | CGTTGAGCACGTCTGG | +C*+G*+T*T*G*A*G*C*A*C*G*T*C*+T*+G*+G |
| 168 | A24092Hi | CACACGTCCAGTTCTCA | +C*+A*+C*A*C*G*T*C*C*A*G*T*T*C*+T*+C*+A |
| 169 | A24093Hi | GCGGATCGGTCAGGAGT | +G*+C*+G*G*A*T*C*G*G*T*C*A*G*G*+A*+G*+T |
| 170 | A24094Hi | TACCACACACCGTCCTG | +T*+A*+C*C*A*C*A*C*A*C*C*G*T*C*+C*+T*+G |
| 171 | A24095Hi | CCTTGTACCACACACCG | +C*+C*+T*T*G*T*A*C*C*A*C*A*C*A*+C*+C*+G |
| 172 | A24096Hi | TTGTACCACACACCGT | +T*+T*+G*T*A*C*C*A*C*A*C*A*C*+C*+G*+T |
| 173 | A24097Hi | CTTAACAGTGGATGACC | +C*+T*+T*A*A*C*A*G*T*G*G*A*T*G*+A*+C*+C |
| 174 | A24098He | CGAGGACGGTTTTTAT | +C*+G*+A*+G*G*A*C*G*G*T*T*T*T*+T*+A*+T |
| 175 | A24099He | AGGATCCGCTCAGCTCG | +A*+G*G*A*T*C*C*G*C*T*C*A*G*C*+T*+C*+G |
| 176 | A24100He | TCGTGTGAGGATCCGCT | +T*+C*+G*T*G*T*G*A*G*G*A*T*C*C*+G*+C*+T |
| 177 | A24101He | AATCGGATCACAGTCGT | +A*+A*+T*C*G*G*A*T*C*A*C*A*G*T*+C*+G*+T |
| 178 | A24102He | TAGCACGGCGGTGGCGG | +T*+A*+G*C*A*C*G*G*C*G*G*T*G*G*+C*+G*+G |
| 179 | A24103He | TAGCACGGCGGTGGCG | +T*+A*+G*C*A*C*G*G*C*G*G*T*G*+G*+C*+G |
| 180 | A24104He | GCGGCGACTTGGACTG | +G*+C*+G*G*C*G*A*C*T*T*G*G*A*+C*+T*+G |
| 181 | A24105He | GGACGCAAAGCGCGGC | +G*+G*+A*C*G*C*A*A*A*G*C*G*C*+G*+G*+C |
| 182 | A24106He | AGGACGCAAAGCGCGG | +A*+G*+G*A*C*G*C*A*A*A*G*C*G*+C*+G*+G |
| 183 | A24107He | GCGGCTGACATTGTGAG | +G*+C*+G*G*C*T*G*A*C*A*T*T*G*T*+G*+A*+G |
| 184 | A24108He | ATAGGCCGTGTCCTCGC | +A*+T*+A*G*G*C*C*G*T*G*T*C*C*T*+C*+G*+C |
| 185 | A24109He | TATAGGCCGTGTCCTCG | +T*+A*+T*A*G*G*C*C*G*T*G*T*C*C*+T*+C*+G |
| 186 | A24110He | AGTACTGGCCGTTGAGG | +A*+G*+T*A*C*T*G*G*C*C*G*T*T*G*+A*+G*+G |
| 187 | A24111He | GAAGTACTGGCCGTTGA | +G*+A*+A*G*T*A*C*T*G*G*C*C*G*T*+T*+G*+A |
| 188 | A24112He | TCTTAAGCTTCTGCCGC | +T*+C*+T*T*A*A*G*C*T*T*C*T*G*C*+C*+G*+C |
| 189 | A24113He | AGTCACCGTCTTTCGTG | +A*+G*+T*C*A*C*C*G*T*C*T*T*T*C*+G*+T*+G |
| 190 | A24114He | CGCCATTGAGGCCAGTC | +C*+G*+C*C*A*T*T*G*A*G*G*C*C*A*+G*+T*+C |
| 191 | A24115He | CTGAGTCCGCCATTGAG | +C*+T*+G*A*G*T*C*C*G*C*C*A*T*T*+G*+A*+G |
| 192 | A24116He | ATTGGCGCCTGCTTGTG | +A*+T*+T*G*G*C*G*C*C*T*G*C*T*T*+G*+T*+G |
| 193 | A24117He | ATACCATTGGCGCCTGC | +A*+T*+A*C*C*A*T*T*G*G*C*G*C*C*+T*+G*+C |
| 190 | A24118He | CGCCATTGAGGCCAGTC | +C*+G*+C*C*A*T*T*G*A*G*G*C*C*A*+G*+T*+C |
| 194 | A24119He | TGAGTCCGCCATTGAGG | +T*+G*+A*G*T*C*C*G*C*C*A*T*T*G*+A*+G*+G |
| 191 | A24120He | CTGAGTCCGCCATTGAG | +C*+T*+G*A*G*T*C*C*G*C*C*A*T*T*+G*+A*+G |
| 195 | A24121He | ACTGAGTCCGCCATTGA | +A*+C*+T*G*A*G*T*C*C*G*C*C*A*T*+T*+G*+A |
| 196 | A24122HMe | TGACTGAGTCCGCCATT | +T*+G*+A*C*T*G*A*G*T*C*C*G*C*C*+A*+T*+T |
| 197 | A24123He | TGTGACTGAGTCCGCCA | +T*+G*+T*G*A*C*T*G*A*G*T*C*C*G*+C*+C*+A |
| 46 | A24124He | ATGTGACTGAGTCCGCC | +A*+T*+G*T*G*A*C*T*G*A*G*T*C*C*+G*+C*+C |
| 198 | A24125He | CCCCGTCAGTCAATGTG | +C*+C*+C*C*G*T*C*A*G*T*C*A*A*T*+G*+T*+G |
| 199 | A24126He | GGTCCCCGTCAGTCAAT | +G*+G*+T*C*C*C*C*G*T*C*A*G*T*C*+A*+A*+T |
| 57 | Neg1 | | +C*+G*+T*T*T*A*G*G*C*T*A*T*G*T*A*+C*+T*+T |
| 249 | R01002 | | +T*+A*+C*G*C*G*C*G*G*T*T*G*T*+T*+T*+A |
| 250 | R01009 | | +T*+T*+A*G*C*G*C*G*C*G*A*A*T*+A*+T*+G |
| 251 | R01014 | | +C*+G*+A*A*T*A*A*C*C*G*T*C*G*T*+G*+T*+T |
| 252 | R01019 | | +G*+A*+C*T*C*G*T*T*A*A*A*C*C*G*+A*+T*+A |

Oligonucleotides of Table 3 hybridize particularly with mRNA and/or pre-m RNA of mouse ANGPTL4:

TABLE 3
List of antisense oligonucleotides hybridizing with
mouse ANGPLT4 mRNA and/or pre-mRNA for example of
SEQ ID NO. 58 and/or SEQ ID NO. 59;
Neg1 is an oligonucleotide representing a negative control which is not
hybridizing with ANGPTL4 of SEQ ID NO. 58 or SEQ ID NO. 59.
Oligonucleotides primarily hybridizing with mouse ANGPLT4 mRNA are
indicated by “M”, and oligonucleotides primarily hybridizing with mouse
ANGPLT4 pre-mRNA are indicated by “Mi” as the oligonucleotides
hybridize with an intron.

| Seq | | Antisense | Antisense Sequence 5′-3′ with |
|---|---|---|---|
| ID | Name | Sequence 5′-3′ | PTO (*) and LNA (+) |
| 60 | A24001M | GTTGCACCTAAAGCC | +G*+T*+T*G*C*A*C*C*T*A*A*A*+G*+C*+C |
| 61 | A24002M | ACGGTTGCACCTAAAGC | +A*+C*+G*G*T*T*G*C*A*C*C*T*A*A*+A*+G*+C |
| 62 | A24003M | ACGGTTGCACCTAAAG | +A*+C*+G*G*T*T*G*C*A*C*C*T*A*+A*+A*+G |
| 63 | A24004M | TCACGGTTGCACCTAA | +T*+C*+A*C*G*G*T*T*G*C*A*C*C*+T*+A*+A |
| 64 | A24005M | TTCACGGTTGCACCTA | +T*+T*+C*A*C*G*G*T*T*G*C*A*C*+C*+T*+A |
| 65 | A24006M | ATAAGCGTTTCACGGTT | +A*+T*+A*A*G*C*G*T*T*T*C*A*C*G*+G*+T*+T |
| 66 | A24007M | CATAAGCGTTTCACGGT | +C*+A*+T*A*A*G*C*G*T*T*T*C*A*C*+G*+G*+T |
| 67 | A24008M | CGTAGCTCATAAGCGTT | +C*+G*+T*A*G*C*T*C*A*T*A*A*G*C*+G*+T*+T |
| 68 | A24009M | GCTAGGACTCCGGAAC | +G*+C*+T*A*G*G*A*C*T*C*C*G*G*+A*+A*+C |
| 69 | A24010M | ACGCTAGGACTCCGGAA | +A*+C*+G*C*T*A*G*G*A*C*T*C*C*G*+G*+A*+A |
| 70 | A24011M | AACGCTAGGACTCCG | +A*+A*+C*G*C*T*A*G*G*A*C*T*+C*+C*+G |
| 71 | A24012M | AGCGCATGATTCTGG | +A*G*+C*G*C*A*T*G*A*T*T*C*+T*G*+G |
| 72 | A24013M | CGGAGCGCAGCGCATGA | +C*G*+G*A*G*C*G*C*A*G*C*G*C*A*+T*G*+A |
| 73 | A24014M | AGCCGCGCATAGCACCA | +A*+G*+C*C*G*C*G*C*A*T*A*G*C*A*+C*+C*+A |
| 74 | A24015M | TAGCCGCGCATAGCAC | +T*+A*+G*C*C*G*C*G*C*A*T*A*G*+C*+A*+C |
| 75 | A24016M | AGTAGCCGCGCATAGCA | +A*+G*+T*A*G*C*C*G*C*G*C*A*T*A*+G*+C*+A |
| 76 | A24017M | CAGTAGCCGCGCATAGC | +C*+A*+G*T*A*G*C*C*G*C*G*C*A*T*+A*+G*+C |
| 77 | A24018M | CAGTAGCCGCGCATAG | +C*+A*+G*T*A*G*C*C*G*C*G*C*A*+T*+A*+G |
| 78 | A24019M | GCAGTAGCCGCGCATA | +G*+C*+A*G*T*A*G*C*C*G*C*G*C*+A*+T*+A |
| 79 | A24020M | CCTTGCGCGCTCAAA | +C*+C*T*T*G*C*G*C*G*C*T*C*+A*A*+A |
| 80 | A24021M | GATGCAAAGCGCGGTG | +G*+A*+T*G*C*A*A*A*G*C*G*C*G*+G*+T*+G |
| 81 | A24022M | AGGATGCAAAGCGCGGT | +A*+G*+G*A*T*G*C*A*A*A*G*C*G*C*+G*+G*+T |
| 82 | A24023M | CCACGTGTTCGCGCAG | +C*+C*+A*C*G*T*G*T*T*C*G*C*G*+C*+A*+G |
| 83 | A24024M | CTCCACGTGTTCGCGCA | +C*+T*+C*C*A*C*G*T*G*T*T*C*G*C*+G*+C*+A |
| 84 | A24025M | TGCGCTCCACGTGTTCG | +T*+G*+C*G*C*T*C*C*A*C*G*T*G*T*+T*+C*+G |
| 85 | A24026M | TGCGCTCCACGTGTTC | +T*+G*+C*G*C*T*C*C*A*C*G*T*G*+T*+T*+C |
| 86 | A24027M | TGCGCTCCACGTGTT | +T*+G*+C*G*C*T*C*C*A*C*G*T*+G*+T*+T |
| 87 | A24028M | ATGCGGCGCTCCAGCG | +A*+T*G*C*G*G*C*G*C*T*C*C*A*G*C*+G |
| 88 | A24029M | CTGACAAGCGTTACCAC | +C*+T*+G*A*C*A*A*G*C*G*T*T*A*C*+C*+A*+C |
| 89 | A24030M | CCTGACAAGCGTTACCA | +C*+C*+T*G*A*C*A*A*G*C*G*T*T*A*+C*+C*+A |
| 90 | A24031M | CCTGACAAGCGTTAC | +C*+C*+T*G*A*C*A*A*G*C*G*T*+T*+A*+C |
| 91 | A24032M | ATTGTCTAGGTGCGTG | +A*+T*+T*G*T*C*T*A*G*G*T*G*C*+G*+T*+G |
| 92 | A24033M | CCATTGTCTAGGTGCGT | +C*+C*+A*T*T*G*T*C*T*A*G*G*T*G*+C*+G*+T |
| 93 | A24034M | CGTTCAGGCGTCTCTGA | +C*G*+T*T*C*A*G*G*C*G*T*C*T*C*+T*G*+A |
| 94 | A24035M | AGAGCCGTTCAGGCGT | +A*G*+A*G*C*C*G*T*T*C*A*G*G*+C*G*+T |
| 95 | A24036M | GCTCATTGGCCGTGG | +G*C*+T*C*A*T*T*G*G*C*C*G*+T*G*+G |
| 96 | A24037M | AGTGGAAGTATTGTCCA | +A*+G*T*G*G*A*A*G*T*A*T*T*G*T*+C*C*+A |
| 97 | A24038M | TACGCTCCTGCCGTTGC | +T*+A*+C*G*C*T*C*C*T*G*C*C*G*T*+T*+G*+C |
| 98 | A24039M | TTACGCTCCTGCCGTTG | +T*+T*+A*C*G*C*T*C*C*T*G*C*C*G*+T*+T*+G |
| 99 | A24040M | TTTTTACGCTCCTGCCG | +T*+T*+T*T*T*A*C*G*C*T*C*C*T*G*+C*+C*+G |
| 100 | A24041M | TTTTACGCTCCTGCC | +T*+T*+T*T*A*C*G*C*T*C*C*T*+G*+C*+C |
| 101 | A24042M | AGAGGATAGTAGCGGCC | +A*+G*A*G*G*A*T*A*G*T*A*G*C*G*G*+C*+C |
| 102 | A24043M | ACAAGACGCAGATAGCC | +A*+C*+A*A*G*A*C*G*C*A*G*A*T*A*+G*+C*+C |
| 103 | A24044M | GGCGAGAAGTGATATTC | +G*+G*+C*G*A*G*A*A*G*T*G*A*T*A*+T*+T*+C |
| 104 | A24045M | TAGGCGAGAAGTGATAT | +T*+A*+G*G*C*G*A*G*A*A*G*T*G*A*+T*+A*+T |
| 105 | A24046M | GTAGGCGAGAAGTGAT | +G*+T*+A*G*G*C*G*A*G*A*A*G*T*+G*+A*+T |
| 106 | A24047M | GAGTCCGCCATTAAGG | +G*+A*+G*T*C*C*G*C*C*A*T*T*A*+A*+G*+G |
| 107 | A24048M | ACTGAGTCCGCCATTAA | +A*+C*+T*G*A*G*T*C*C*G*C*C*A*T*+T*+A*+A |
| 108 | A24049M | ATATGACTGAGTCCGCC | +A*+T*+A*T*G*A*C*T*G*A*G*T*C*C*+G*+C*+C |
| 109 | A24050M | AATATGACTGAGTCCGC | +A*+A*+T*A*T*G*A*C*T*G*A*G*T*+C*+C*+G*+C |
| 110 | A24051M | CGCTAGGACTCCGGAA | +C*+G*C*T*A*G*G*A*C*T*C*C*G*+G*+A*+A |
| 111 | A24052M | AACGCTAGGACTCCGGA | +A*+A*C*G*C*T*A*G*G*A*C*T*C*C*G*+G*+A |
| 112 | A24053M | AACGCTAGGACTCCGG | +A*+A*+C*G*C*T*A*G*G*A*C*T*C*C*+G*+G |
| 113 | A24054M | CAACGCTAGGACTCCG | +C*+A*+A*+C*G*C*T*A*G*G*A*C*T*+C*+C*+G |
| 114 | A24055M | GCAACGCTAGGACTCCG | +G*+C*+A*+A*C*G*C*T*A*G*G*A*C*T*+C*+C*+G |
| 115 | A24056M | GTAGCCGCGCATAGC | +G*+T*+A*G*C*C*G*C*G*C*A*T*+A*+G*+C |
| 116 | A24057M | AGTAGCCGCGCATAGC | +A*+G*+T*A*G*C*C*G*C*G*C*A*T*+A*+G*+C |
| 117 | A24058M | AGTAGCCGCGCATAG | +A*+G*+T*A*G*C*C*G*C*G*C*A*+T*+A*+G |
| 118 | A24059M | GCAGTAGCCGCGCATAG | +G*+C*+A*G*T*A*G*C*C*G*C*G*C*A*+T*+A*+G |
| 118 | A24060M | GCAGTAGCCGCGCATAG | +G*+C*+A*+G*T*A*G*C*C*G*C*G*C*A*+T*+A*+G |
| 119 | A24061M | CAGTAGCCGCGCATA | +C*+A*+G*T*A*G*C*C*G*C*G*C*+A*+T*+A |
| 120 | A24062M | CCGCAGTAGCCGCGCAT | +C*C*G*C*A*G*T*A*G*C*C*G*C*G*+C*+A*+T |
| 121 | A24063M | CCTTGCGCGCTCAAAA | +C*C*+T*T*G*C*G*C*G*C*T*C*A*A*+A*+A |
| 122 | A24064M | CCTTGCGCGCTCAAAA | +C*+C*T*T*G*C*G*C*G*C*T*C*A*+A*+A*+A |
| 79 | A24065M | CCTTGCGCGCTCAAA | +C*+C*+T*T*G*C*G*C*G*C*T*C*+A*+A*+A |
| 123 | A24066M | ATGCAAAGCGCGGTGG | +A*+T*+G*C*A*A*A*G*C*G*C*G*+G*+T*+G*+G |
| 124 | A24067M | ATGCAAAGCGCGGTG | +A*+T*+G*C*A*A*A*G*C*G*C*G*+G*+T*+G |
| 125 | A24068M | CGCTCCACGTGTTCG | +C*G*+C*T*C*C*A*C*G*T*G*T*+T*+C*+G |
| 126 | A24069M | GCGCTCCACGTGTTCG | +G*+C*G*C*T*C*C*A*C*G*T*G*T*+T*+C*+G |
| 127 | A24070M | TGAGTCCGCCATTAAGG | +T*+G*+A*+G*T*C*C*G*C*C*A*T*T*A*+A*+G*+G |
| 128 | A24071M | ATGACTGAGTCCGCCA | +A*T*G*A*C*T*G*A*G*T*C*C*G*+C*+C*+A |
| 129 | A24072M | TATGACTGAGTCCGCC | +T*+A*+T*G*A*C*T*G*A*G*T*C*C*+G*+C*+C |
| 130 | A24073M | AATATGACTGAGTCCGC | +A*+A*+T*+A*T*G*A*C*T*G*A*G*T*+C*+C*+G*+C |
| 131 | A24074M | AATATGACTGAGTCCG | +A*+A*+T*A*T*G*A*C*T*G*A*G*T*+C*C*+G |
| 132 | A24075Mi | CCTAGTAGATGCGCCTA | +C*+C*+T*A*G*T*A*G*A*T*G*C*G*C*C*+T*+A |
| 133 | A24076Mi | TCGTTAGTCTAAGTAGA | +T*+C*+G*T*T*A*G*T*C*T*A*A*G*T*+A*+G*+A |
| 134 | A24077Mi | AGTACTAGACTCGTTAG | +A*+G*+T*A*C*T*A*G*A*C*T*C*G*T*+T*+A*+G |
| 135 | A24078Mi | TCGGCAACCTCCTCTTA | +T*+C*+G*G*C*A*A*C*C*T*C*C*T*C*T*+T*+A |
| 136 | A24079Mi | GCTTTATGTCGGCAACC | +G*+C*+T*T*T*A*T*G*T*C*G*G*C*A*+A*+C*+C |
| 137 | A24080Mi | TTGACTCATGCCATAAC | +T*+T*+G*A*C*T*C*A*T*G*C*C*A*T*+A*+A*+C |
| 138 | A24081Mi | GTTAACGGCTAATAAGA | +G*+T*+T*A*A*C*G*G*C*T*A*A*T*A*+A*+G*+A |
| 139 | A24082Mi | TCTGGTTAACGGCTAAT | +T*+C*+T*G*G*T*T*A*A*C*G*G*C*T*+A*+A*+T |
| 140 | A24083Mi | ACTTTAGCTCCTTATGA | +A*+C*+T*T*T*A*G*C*T*C*C*T*T*A*+T*+G*+A |
| 141 | A24084Mi | GATCGAAACTGTTATGT | +G*+A*+T*C*G*A*A*A*C*T*G*T*T*+A*+T*+G*+T |
| 141 | A24085Mi | AGAACCTATGCCTGCCG | +A*+G*+A*A*C*C*T*A*T*G*C*C*T*G*C*+C*+G |
| 142 | A24086Mi | TCCTCTCATCCAATCGG | +T*+C*+C*T*C*T*C*A*T*C*C*A*A*T*+C*+G*+G |
| 143 | A24087Mi | ACGGCTATGTCTGTTAC | +A*+C*+G*G*C*T*A*T*G*T*C*T*G*T*+T*+A*+C |
| 144 | A24088Mi | GGACCTGTAACCACCTA | +G*+G*+A*C*C*T*G*T*A*A*C*C*A*C*+C*+T*+A |
| 145 | A24089Mi | GTATTGTCGCTGATGAA | +G*+T*+A*T*T*G*T*C*G*C*T*G*A*T*+G*+A*+A |
| 146 | A24090Mi | GATTTGCCTAAACTCGT | +G*+A*+T*T*T*G*C*C*T*A*A*A*C*T*+C*+G*+T |
| 147 | A24091Mi | GAGCTTGCGATGCCTGT | +G*A*+G*C*T*T*G*C*G*A*T*G*C*C*T*+G*+T |
| 148 | A24092Mi | GTAGATGACTAGGCCTG | +G*+T*+A*G*A*T*G*A*C*T*A*G*G*C*C*+T*+G |
| 149 | A24093Mi | TCAATGGAAGCGCTTTA | +T*+C*+A*A*T*G*G*A*A*G*C*G*C*T*+T*+T*+A |
| 150 | A24094Mi | GTAGCAGACTTGCACTA | +G*+T*+A*G*C*A*G*A*C*T*T*G*C*A*+C*+T*+A |
| 151 | A24095Mi | CTCATGTTAGGTAGGTT | +C*+T*+C*A*T*G*T*T*A*G*G*T*A*G*+G*+T*+T |
| 152 | A24096Mi | ACGTGGCCAAAGACAAT | +A*+C*+G*T*G*G*C*C*A*A*A*G*A*C*+A*+A*+T |
| 57 | Neg1 | | +C*+G*+T*T*T*A*G*G*C*T*A*T*G*T*A*+C*+T*+T |

Oligonucleotides of Table 4 also hybridize particularly with mRNA and/or pre-m RNA of mouse 10 ANGPTL4:

TABLE 4
List of antisense oligonucleotides hybridizing with
mouse ANGPLT4 mRNA and/or pre-mRNA for example of
SEQ ID NO. 58 and/or SEQ ID NO. 59;
Neg1, R01002, R01009, R01014 and R01019 are oligonucleotides representing
negative controls which are not hybridizing with ANGPTL4 of
SEQ ID NO. 58 or SEQ ID NO. 59.
Oligonucleotides primarily hybridizing with mouse ANGPLT4 mRNA are
indicated by “M”, and oligonucleotides primarily hybridizing with mouse
ANGPLT4 pre-mRNA are indicated by “Mi” as the oligonucleotides hybridize
with an intron.

| Seq | | Antisense | Antisense Sequence 5′-3′ with |
|---|---|---|---|
| ID | Name | Sequence 5′-3′ | PTO (*) and LNA (+) |
| 200 | A24097M | TTCACGGTTGCACCTAA | +T*+T*+C*A*C*G*G*T*T*G*C*A*C*C*+T*+A*+A |
| 201 | A24098M | ATAAGCGTTTCACGGT | +A*+T*+A*A*G*C*G*T*T*T*C*A*C*+G*+G*+T |
| 202 | A24099M | GCTAGGACTCCGGAACG | +G*+C*+T*A*G*G*A*C*T*C*C*G*G*A*+A*+C*+G |
| 203 | A24100M | CAGCAACGCTAGGACTC | +C*+A*+G*C*A*A*C*G*C*T*A*G*G*A*+C*+T*+C |
| 204 | A24101M | GTGCAGCAACGCTAGGA | +G*+T*+G*C*A*G*C*A*A*C*G*C*T*A*+G*+G*+A |
| 205 | A24102M | CCGCGCATAGCACCAGG | +C*+C*+G*C*G*C*A*T*A*G*C*A*C*C*+A*+G*+G |
| 206 | A24103M | ATGCAAAGCGCGGTGGC | +A*+T*+G*C*A*A*A*G*C*G*C*G*G*T*+G*+G*+C |
| 207 | A24104M | CCACGTGTTCGCGCAGC | +C*+C*+A*C*G*T*G*T*T*C*G*C*G*C*+A*+G*+C |
| 208 | A24105M | AGCGTTACCACAGGCAG | +A*+G*+C*G*T*T*A*C*C*A*C*A*G*G*+C*+A*+G |
| 209 | A24106M | TCTATCCTCGGAGTCTT | +T*+C*+T*A*T*C*C*T*C*G*G*A*G*T*+C*+T*+T |
| 210 | A24107M | CTCGAAGTCTTGTCTAC | +C*+T*+C*G*A*A*G*T*C*T*T*G*T*C*+T*+A*+C |
| 211 | A24108M | GAAGTATTGTCCATTGA | +G*+A*+A*G*T*A*T*T*G*T*C*C*A*T*+T*+G*+A |
| 212 | A24109M | CAGAGGATAGTAGCGGC | +C*+A*+G*A*G*G*A*T*A*G*T*A*G*C*+G*+G*+C |
| 213 | A24110M | CAGTCTCTCCAGTTACG | +C*+A*+G*T*C*T*C*T*C*C*A*G*T*T*+A*+C*+G |
| 214 | A24111M | GCGAGAAGTGATATTC | +G*+C*+G*A*G*A*A*G*T*G*A*T*A*+T*+T*+C |
| 107 | A24112M | ACTGAGTCCGCCATTAA | +A*+C*+T*G*A*G*T*C*C*G*C*C*A*T*+T*+A*+A |
| 108 | A24113M | ATATGACTGAGTCCGCC | +A*+T*+A*T*G*A*C*T*G*A*G*T*C*C*+G*+C*+C |
| 215 | A24114M | TCAATATGACTGAGTCC | +T*+C*+A*A*T*A*T*G*A*C*T*G*A*G*+T*+C*+C |
| 216 | A24115M | TCCAGTCAGTCAATATG | +T*+C*+C*A*G*T*C*A*G*T*C*A*A*T*+A*+T*+G |
| 217 | A24116M | CTCCAGTCAGTCAATAT | +C*+T*+C*C*A*G*T*C*A*G*T*C*A*A*+T*+A*+T |
| 218 | A24117Mi | GAGTCCTAGTAGATGCG | +G*+A*+G*T*C*C*T*A*G*T*A*G*A*T*+G*+C*+G |
| 219 | A24118Mi | GTTAGTCTAAGTAGAGT | +G*+T*+T*A*G*T*C*T*A*A*G*T*A*G*+A*+G*+T |
| 220 | A24119Mi | AGTACTAGACTCGTTA | +A*+G*+T*A*C*T*A*G*A*C*T*C*G*+T*+T*+A |
| 221 | A24120Mi | GCAACCTCCTCTTATTC | +G*+C*+A*A*C*C*T*C*C*T*C*T*T*A*+T*+T*+C |
| 222 | A24121Mi | AAGATATGCAAGGCTAG | +A*+A*+G*A*T*A*T*G*C*A*A*G*G*C*+T*+A*+G |
| 223 | A24122Mi | GACTCATGCCATAACAA | +G*+A*+C*T*C*A*T*G*C*C*A*T*A*A*+C*+A*+A |
| 224 | A24123Mi | GTGGACCTGACAAGAAG | +G*+T*+G*G*A*C*C*T*G*A*C*A*A*G*+A*+A*+G |
| 225 | A24124Mi | AACGGCTAATAAGATTT | +A*+A*+C*G*G*C*T*A*A*T*A*A*G*A*+T*+T*+T |
| 226 | A24125Mi | GTTCTGGTTAACGGCTA | +G*+T*+T*C*T*G*G*T*T*A*A*C*G*G*+C*+T*+A |
| 227 | A24126Mi | GGTGTGCTTACTCTGGT | +G*+G*+T*G*T*G*C*T*T*A*C*T*C*T*+G*+G*+T |
| 228 | A24127Mi | CCTAGAAATTGTGATCG | +C*+C*+T*A*G*A*A*A*T*T*G*T*G*A*+T*+C*+G |
| 229 | A24128Mi | AACGAATAGGCATGAAC | +A*+A*+C*G*A*A*T*A*G*G*C*A*T*G*+A*+A*+C |
| 230 | A24129Mi | ACTTTCACCTAGTTGGC | +A*+C*+T*T*T*C*A*C*C*T*A*G*T*T*+G*+G*+C |
| 231 | A24130Mi | CTCTACTTGGCTAGGCT | +C*+T*+C*T*A*C*T*T*G*G*C*T*A*G*+G*+C*+T |
| 232 | A24131Mi | GGTACTCTGAATTAGTA | +G*+G*+T*A*C*T*C*T*G*A*A*T*T*A*+G*+T*+A |
| 233 | A24132Mi | TGTAACCACCTAAAGCC | +T*+G*+T*A*A*C*C*A*C*C*T*A*A*A*+G*+C*+C |
| 234 | A24133Mi | AGGTATTGTCGCTGATG | +A*+G*+G*T*A*T*T*G*T*C*G*C*T*G*+A*+T*+G |
| 235 | A24134Mi | GAACAGAGGTATTGTCG | +G*+A*+A*C*A*G*A*G*G*T*A*T*T*G*+T*+C*+G |
| 236 | A24135Mi | CATGTATCACACCTTCC | +C*+A*+T*G*T*A*T*C*A*C*A*C*C*T*+T*+C*+C |
| 237 | A24136Mi | TAAACTCGTTCCTGCCT | +T*+A*+A*A*C*T*C*G*T*T*C*C*T*G*+C*+C*+T |
| 238 | A24137Mi | GCCTAAACTCGTTCCTG | +G*+C*+C*T*A*A*A*C*T*C*G*T*T*C*+C*+T*+G |
| 239 | A24138Mi | ATCTCAGGAGCTTATAC | +A*+T*+C*T*C*A*G*G*A*G*C*T*T*A*+T*+A*+C |
| 240 | A24139Mi | ACAAGCTGCATAATAGG | +A*+C*+A*A*G*C*T*G*C*A*T*A*A*T*+A*+G*+G |
| 241 | A24140Mi | TATCACTGAGCTTGCGA | +T*+A*+T*C*A*C*T*G*A*G*C*T*T*G*+C*+G*+A |
| 242 | A24141Mi | AATGGAAGCGCTTTACC | +A*+A*+T*G*G*A*A*G*C*G*C*T*T*T*+A*+C*+C |
| 243 | A24142Mi | TAGCAGACTTGCACTAT | +T*+A*+G*C*A*G*A*C*T*T*G*C*A*C*+T*+A*+T |
| 244 | A24143Mi | AGGCTCAACTCTCGCAC | +A*+G*+G*C*T*C*A*A*C*T*C*T*C*G*+C*+A*+C |
| 245 | A24144Mi | AAGAGCTAGTACTGTAG | +A*+A*+G*A*G*C*T*A*G*T*A*C*T*G*+T*+A*+G |
| 246 | A24145Mi | TATAATTTGATCCTGAC | +T*+A*+T*A*A*T*T*T*G*A*T*C*C*T*+G*+A*+C |
| 247 | A24146Mi | GGTTCTCTGCCAAATGA | +G*+G*+T*T*C*T*C*T*G*C*C*A*A*A*+T*+G*+A |
| 248 | A24147Mi | CGTGGCCAAAGACAATT | +C*+G*+T*G*G*C*C*A*A*A*G*A*C*A*+A*+T*+T |
| 151 | A24148Mi | CTCATGTTAGGTAGGTT | +C*+T*+C*A*T*G*T*T*A*G*G*T*A*G*+G*+T*+T |
| 57 | Neg1 | | +C*+G*+T*T*T*A*G*G*C*T*A*T*G*T*A*+C*+T*+T |
| 249 | R01002 | | +T*+A*+C*G*C*G*C*G*G*T*T*G*T*+T*+T*+A |
| 250 | R01009 | | +T*+T*+A*G*C*G*C*G*C*G*A*A*T*+A*+T*+G |
| 251 | R01014 | | +C*+G*+A*A*T*A*A*C*C*G*T*C*G*T*+G*+T*+T |
| 252 | R01019 | | +G*+A*+C*T*C*G*T*T*A*A*A*C*C*G*+A*+T*+A |

The oligonucleotides hybridize for example within a hybridizing active area which is an area enriched for ASOs with high activity. The hybridizing active area is for example one or more region(s) on the ANGPTL4 mRNA, e.g., of SEQ ID NO.1 and/or the ANGPTL4 pre-mRNA, e.g., of SEQ ID NO.2, where hybridization with an oligonucleotide highly likely results in a potent knockdown of the ANGPTL4 expression. In the present invention surprisingly several hybridizing active areas were identified for example selected from hybridizing active areas for example selected from position 1732-1759 (e.g., A24044He, SEQ ID NO.47; A24076He, SEQ ID NO.47) of SEQ ID NO. 1 and/or from position 6603-6631 (e.g., A24022Hi, SEQ ID NO.25; A24023Hi, SEQ ID NO.26; A24071Hi, SEQ ID NO.54) of SEQ ID NO.2. Further hybridizing active areas are from position 234-261 (e.g. A24102He, SEQ ID NO.178; A24103He, SEQ ID NO.179) and/or from position 1264-1293 (e.g. A24110He, SEQ ID NO.186; A24111He, SEQ ID NO.187) of human SEQ ID NO. 1 and/or from position 2800-2872 (e.g. A24083Hi, SEQ ID NO. 159; A24085Hi, SEQ ID NO.161; A24086Hi, SEQ ID NO.162; A24087Hi, SEQ ID NO. 163) and/or from position 3415-3442 (e.g. A24089Hi, SEQ ID NO.165) and/or from position 4968-4994 (e.g. A24097Hi, SEQ ID NO. 173) of human SEQ ID NO.2. Hybridizing active areas on mouse SEQ ID NO.58 or SEQ ID NO.59 are for example from position 137-163 (e.g. A24054M, SEQ ID NO.113) and/or from position 215-299 (e.g. A24018M, SEQ ID NO.77; A24019M, SEQ ID NO.78; A24020M, SEQ ID NO.79; A24021M, SEQ ID NO.80; A24065M, SEQ ID NO.79) and/or from position 1343-1371 (e.g. A24042M, SEQ ID NO.101; A24109M, SEQ ID NO.212) and/or from position 1738-1771 (e.g. A24047M, SEQ ID NO.106; A24070M, SEQ ID NO. 127; A24072M, SEQ ID NO. 129) of SEQ ID NO.58 and/or from position 1286-1314 (e.g. A24082Mi, SEQ ID NO.139; A24125Mi, SEQ ID NO.226) and/or from position 5485-5511 (e.g. A24095Mi, SEQ ID NO.151; A24148Mi, SEQ ID NO.151) of SEQ ID NO.59.

The oligonucleotide of the present invention inhibits for example at least about 50%, 55%, 60%, 65%, 70%, 75%, 80%, 85%, 90%, 92%, 93%, 94%, 95%, 96%, 97%, 98%, 99% or 100% of ANGPTL4 such as the, e.g., human or mouse, ANGPTL4 expression. The oligonucleotide of the present invention inhibits the expression of ANGPTL4 at a nanomolar or micromolar concentration for example in a concentration range of 0.1 nM to 100 μM, 0.5 nM to 15 nM, 0,6 nM to 10 nM, 1 nM to 10 μM, 5 nM to 5 μM, 10 nM to 1 μM, 15 nM to 950 nM, 20 nM to 900 nM, 25 nM to 850 nM, 30 nM to 800 nM, 35 nM to 750 nM, 40 nM to 700 nM, 45 nM to 650 nM, 50 nM to 500 nM, or 40 nM to 150 nM, or in a concentration of 0.1, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900 or 950 nM, or 1, 10 or 100 μM.

The ANGPTL4 oligonucleotide of the present invention is for example used in a concentration range of 1 nM to 10 μM, 5 nM to 6.6 μM, 10 nM to 5 μM, 15 nM to 3 μM, 20 nM to 2.2 μM, 25 nM to 1 μM, 30 nM to 800 nM, 50 nM to 500 nM, 60 nM to 300 nM, 70 nM to 250 nM, 80 nM to 200 nM, 90 nM to 120 nM, or in a concentration of 1, 1.6, 3, 5, 8, 9, 10, 15, 20, 25, 27, 30, 40, 50, 75, 82, 100, 200, 250, 300, 500, or 740 nM, or 1, 2.2, 3, 5, 6.6 or 10 μM.

The ANGPTL4 oligonucleotide of the present invention is for example administered once or repeatedly, e.g., every 12 h, every 24 h, every 48 h for some weeks, months or years, or it is administered every week, every two weeks, every three weeks or every months or every three or six months.

In some embodiments the present invention refers to a pharmaceutical composition comprising an ANGPTL4 oligonucleotide of the present invention and a pharmaceutically acceptable carrier, excipient and/or dilutant. Optionally, the pharmaceutical composition further comprises a chemotherapeutic, another disease specific active agent such as insulin, angiotensin-converting enzyme inhibitor, angiotensin receptor blocker, another oligonucleotide not of the present invention, an antibody, a HERA fusion protein, a ligand trap, a Fab fragment, a nanobody, a BiTe and/or a small molecule which is for example effective in tumor treatment, treatment of diabetes and its side effects, treatment of a cardiovascular disease, obesity, diabetes type II, hypercholesterolemia such as homozygote familial hypercholesterolemia (HoFH), heterozygote familial hypercholesterolemia (HeFH) or dyslipidemia.

The ANGPTL4 oligonucleotide or the pharmaceutical composition of the present invention is for use in a method of preventing and/or treating a disorder for example a disorder where an ANGPTL4 imbalance is involved. Optionally, the use of the oligonucleotide or the pharmaceutical composition of the present invention in a method of preventing and/or treating a disorder is combined with radiotherapy. The radiotherapy may be further combined with a chemotherapy (e.g., platinum, gemcitabine). The disorder is for example characterized by an ANGPTL4 imbalance, i.e., the ANGPTL4 level is increased in comparison to the level in a normal, healthy cell, tissue, organ or subject. The ANGPTL4 level is for example increased by an increased ANGPTL4 expression and activity, respectively. The ANGPTL4 level is measured by any standard method such as immunohistochemistry, western blot, quantitative real time PCR or QuantiGene assay known to a person skilled in the art.

The ANGPTL4 oligonucleotide or a pharmaceutical composition of the present invention is administered locally or systemically for example orally, sublingually, nasally, subcutaneously, intravenously, intraperitoneally, intramuscularly, intratumoral, intrathecal, transdermal, and/or rectal. Alternatively or in combination ex vivo treated immune cells are administered. The ANGPTL4 oligonucleotide is administered alone or in combination with another ANGPTL4 antisense oligonucleotide of the present invention and optionally in combination with another compound such as another oligonucleotide not of the present invention, an antibody, a HERA fusion protein, a ligand trap, a Fab fragment, a nanobody, a BiTe, a small molecule and/or a chemotherapeutic (e.g., platinum, gemcitabine) and/or another disease specific agent such as insulin, angiotensin-converting enzyme inhibitor, and/or angiotensin receptor blocker.

The oligonucleotide not of the present invention, an antibody, a HERA fusion protein, a ligand trap, a Fab fragment, a nanobody, a BiTe, and/or the small molecule are effective in preventing and/or treating a tumor, influenza infection, diabetes such as diabetes type II and its side effects, a cardiovascular disease, obesity, hypercholesterolemia such as homozygote familial hypercholesterolemia (HoFH), heterozygote familial hypercholesterolemia (HeFH) or dyslipidemia. An ANGPTL4 oligonucleotide or a pharmaceutical composition of the present invention is used for example in a method of preventing and/or treating a solid tumor or a hematologic tumor. Examples of cancers preventable and/or treatable by use of the oligonucleotide or pharmaceutical composition of the present invention are breast cancer, lung cancer, malignant melanoma, lymphoma, skin cancer, bone cancer, prostate cancer, liver cancer, brain cancer, cancer of the larynx, gall bladder, pancreas, testicular, rectum, parathyroid, thyroid, adrenal, neural tissue, head and neck, colon, stomach, bronchi, kidneys, basal cell carcinoma, squamous cell carcinoma, metastatic skin carcinoma, osteo sarcoma, Ewing's sarcoma, reticulum cell sarcoma, liposarcoma, myeloma, giant cell tumor, small-cell lung tumor, islet cell tumor, primary brain tumor, meningioma, acute and chronic lymphocytic and granulocytic tumors, acute and chronic myeloid leukemia, hairy-cell tumor, adenoma, hyperplasia, medullary carcinoma, intestinal ganglioneuromas, Wilm's tumor, seminoma, ovarian tumor, leiomyomater tumor, cervical dysplasia, retinoblastoma, soft tissue sarcoma, malignant carcinoid, topical skin lesion, rhabdomyosarcoma, Kaposi's sarcoma, osteogenic sarcoma, malignant hypercalcemia, renal cell tumor, polycythermia vera, adenocarcinoma, anaplastic astrocytoma, glioblastoma multiforma, leukemia, or epidermoid carcinoma.

Further examples of diseases preventable and/or treatable by use of the ANGPTL4 oligonucleotide or the pharmaceutical composition of the present invention other than cancer are for example diabetes such as diabetes type II and its side effects, a cardiovascular disease, obesity, hypercholesterolemia such as homozygote familial hypercholesterolemia (HoFH), heterozygote familial hypercholesterolemia (HeFH) or dyslipidemia.

In some examples two or more ANGPTL4 oligonucleotides of the present invention are administered together, at the same time point for example in a pharmaceutical composition or separately, or on staggered intervals. In some examples two or more ANGPTL4 oligonucleotides of the present invention are administered together, at the same time point for example in a pharmaceutical composition or separately, or on staggered intervals. In other examples, one or more oligonucleotides of the present invention are administered together with another compound such as another oligonucleotide not of the present invention, an antibody, a HERA fusion protein, a ligand trap, a Fab fragment, a nanobody, a BiTe, a small molecule and/or a chemotherapeutic, at the same time point for example in a pharmaceutical composition or separately, or on staggered intervals.

A subject of the present invention is for example a mammalian, a bird or a fish.

### EXAMPLES

The following examples illustrate different embodiments of the present invention, but the invention is not limited to these examples. The following experiments are performed on cells endogenously expressing ANGPTL4, i.e., the cells do not represent an artificial system comprising transfected reporter constructs. Such artificial systems generally show a higher degree of inhibition and lower IC₅₀ values than endogenous systems which are closer to therapeutically relevant in vivo systems. Further, no transfecting agent is used in the following experiments, i.e., gymnotic delivery is performed. Transfecting agents are known to increase the activity of an oligonucleotide which influences the IC₅₀ value (see for example Zhang et al., Gene Therapy, 2011, 18, 326-333; Stanton et al., Nucleic Acid Therapeutics, Vol. 22, NO. 5, 2012). As artificial systems using a transfecting agent are hard or impossible to translate into therapeutic approaches and no transfection formulation has been approved so far for oligonucleotides, the following experiments are performed without any transfecting agent, except for the experiments of Example 11, where a transfection reagent was used.

### Example 1: 1^{st} Screen of Human ANGPTL4-Specific Antisense Oligonucleotides in HeLa Cells

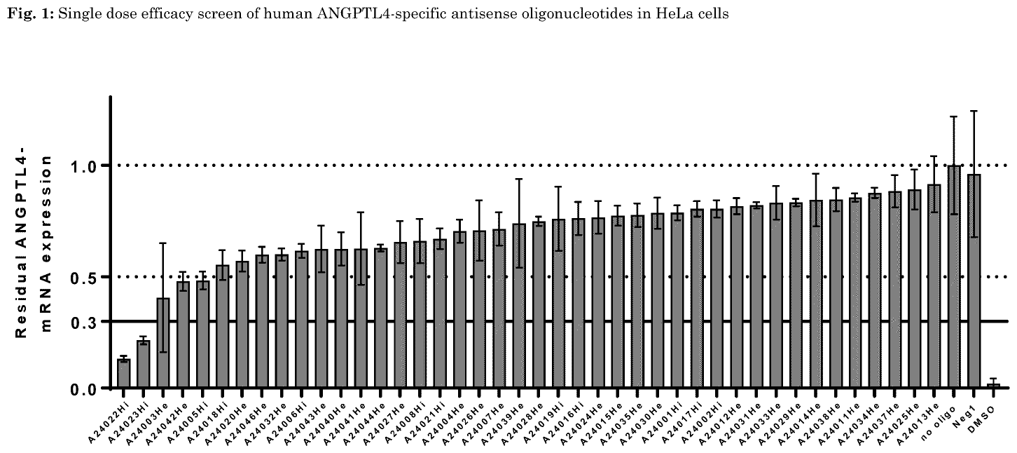

5,000 HeLa cells/well were seeded in 96-well plates and treated with the respective antisense oligonucleotides as indicated in FIG. 1 at a final concentration of 10 μM. To induce ANGPTL4 mRNA expression, cells were simultaneously treated with 1 μM PPARδ (Sigma Aldrich, Cat. No. SML1491). PPARδ used in the example is for example based on a PPARδ stock solution (10 mM) that was prepared by dissolving 5 mg PPARδ (Molecular weight: 453.50) in 1.1 ml DMSO. For final concentration of 1 μM PPARδ, cells seeded in 96-well plates were incubated with 100 μl medium supplemented with 0.01 μl PPARδ stock solution. As a negative control, cells were treated with equal volume of DMSO. Three days after start of treatment, cells were lysed and human HPRT1 as well as human ANGPTL4 mRNA expression was measured using the QuantiGene RNA Singleplex assay (FIG. 1). The QuantiGene Assay used in the examples is for example built upon the branched DNA technology (bDNA), which relies on cooperative hybridization between a target mRNA and a specific probe set (part of QuantiGene Reagent System). The assay is performed according to manufacturer's protocol (Thermo Fisher Scientific) and is used for determination of RNA levels. It combines the QuantiGene Sample Processing Kit that is used for cell lysis and the QuantiGene Reagent System that is used for hybridization, amplification and detection of RNA of interest. The QuantiGene Reagent System is based on an RNA-specific probe set, designed to detect a particular RNA of interest.

ANGPTL4-mRNA expression values were normalized to expression of the housekeeping gene HPRT1. Residual ANGPTL4-mRNA expression relative to mock-treated cells (“no oligo” set as 1) is shown. Solid line and dotted lines indicate 70% and 50% or 0% knockdown efficacy, respectively. Data are represented as mean of triplicate wells+/−SD.

As shown in FIG. 1, two antisense oligonucleotides (A24022Hi (SEQ ID NO.25) and A24023Hi (SEQ ID NO.26)) reduced the normalized ANGPTL4 expression by more than 70% (equivalent to residual mRNA level of <0.3). Further, three antisense oligonucleotides (A24003He (SEQ ID NO.5), A24042He (SEQ ID NO.45), A24005Hi (SEQ ID NO.7)) showed knockdown efficacy between 70% and 50%, while control oligonucleotide (Neg1) did not reduce ANGPTL4 mRNA expression.

### Example 2: 2^{nd} Screen of Human ANGPTL4-Specific Antisense Oligonucleotides in SK-OV3 Cells

5,000 SK-OV3 cells/well were seeded in 96-well plates and treated with the respective ANGPTL4 antisense oligonucleotides at a final concentration of 10 μM. To induce ANGPTL4 mRNA expression, cells were simultaneously treated with 1 μM PPARδ (Sigma Aldrich, cat. no. SML1491; for preparation of 10 mM stock solution see example 1). For final concentration of 1 μM PPARδ, cells seeded in 96-well plates were incubated with 100 μl medium supplemented with 0.01 μl PPARδ stock solution. As a negative control, cells were treated with equal volume of DMSO.

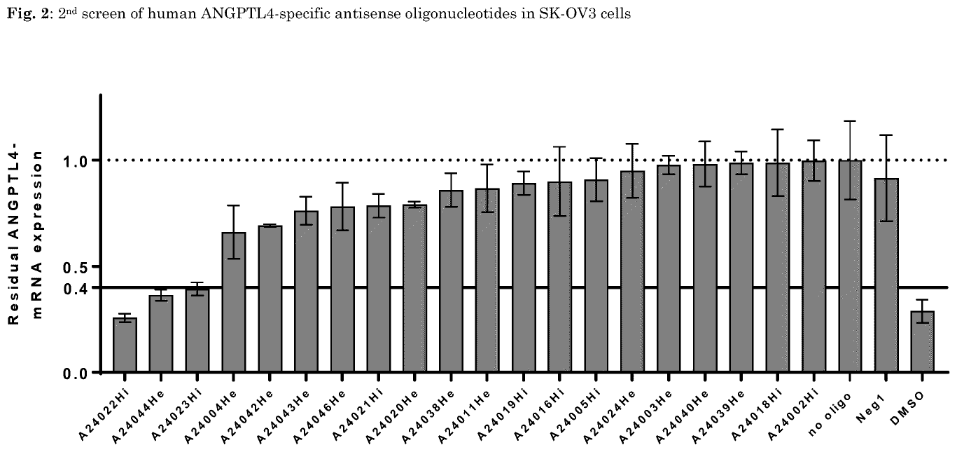

Three days after start of treatment, cells were lysed and human HPRT1 as well as human ANGPTL4 mRNA expression was measured using the QuantiGene RNA Singleplex assay. ANGPTL4-mRNA expression values were normalized to expression of the housekeeping gene HPRT1. Residual ANGPTL4-mRNA expression relative to mock-treated cells (“no oligo” set as 1) is shown in FIG. 2. Solid line and dotted lines indicate 60% and 0% knockdown efficacy, respectively. Data are represented as mean of triplicate wells+/−SD.

Repetition of the ANGPTL4 antisense oligonucleotide screening in SK-OV3 cells resulted in three ANGPTL4 antisense oligonucleotides (A24022Hi (SEQ ID NO.25), A24044He (SEQ ID NO.47), A24023Hi (SEQ ID NO.26)) with more than 60% knockdown efficiency.

### Example 3: 1^{st} Single Dose Efficacy Screen of Further Human ANGPTL4-Specific Antisense Oligonucleotides in HeLa Cells

5,000 HeLa cells/well were seeded in 96-well plates and treated with the most efficient ANGPTL4 antisense oligonucleotides (A24022Hi (SEQ ID NO.25), A24023Hi (SEQ ID NO.26), A24044He (SEQ ID NO.47)) from 1st screenings in HeLa (FIG. 1) and SK-OV3 (FIG. 2) as well as further ANGPTL4-specific antisense oligonucleotides (A24071Hi (SEQ ID NO.54), A24076He (SEQ ID NO.47), A24075He (SEQ ID NO.5), A24073Hi (SEQ ID NO.55), A24065Hi (SEQ ID NO.51), A24067Hi (SEQ ID NO.52), A24077He (SEQ ID NO.47), A24074Hi (SEQ ID NO.56)) at a final concentration of 10 μM. To induce ANGPTL4 mRNA expression, cells were simultaneously treated with 1 μM PPARδ (Sigma Aldrich, cat. no. SML1491; for preparation of 10 mM stock solution see example 1). For final concentration of 1 μM PPARδ, cells seeded in 96-well plates were incubated with 100 μl medium supplemented with 0.01 μl PPARδ stock solution. As a negative control, cells were treated with equal volume of DMSO.

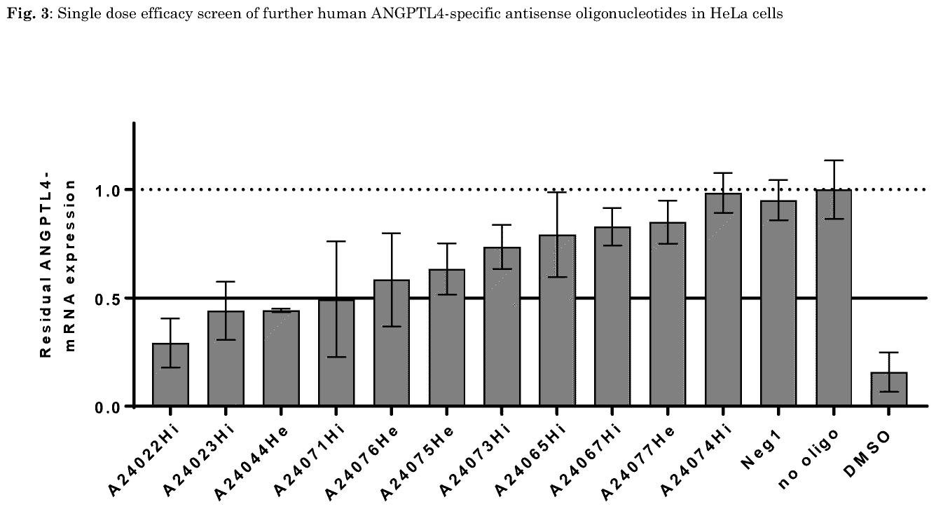

Three days after start of treatment, cells were lysed and human HPRT1 as well as human ANGPTL4 mRNA expression was measured using the QuantiGene RNA Singleplex assay. ANGPTL4-mRNA expression values were normalized to expression of the housekeeping gene HPRT1. Residual ANGPTL4-mRNA expression relative to mock-treated cells (“no oligo” set as 1) is shown in FIG. 3. Solid line and dotted lines indicate 50% and 0% knockdown efficacy, respectively. Data are represented as mean of triplicate wells+/−SD.

As shown in FIG. 3, four of the tested ANGPTL4 antisense oligonucleotides (A24022Hi (SEQ ID NO.25), A24023Hi (SEQ ID NO.26), A24044He (SEQ ID NO.47), A24071Hi (SEQ ID NO.54)) show more than 50% knockdown of ANGPTL4 mRNA (equivalent to residual mRNA level of <0.5), while treatment with Neg1 negative control oligonucleotide did not result in decreased ANGPTL4 mRNA levels.

### Example 4: 2^{nd} Single Dose Efficacy Screen of Further Human ANGPTL4-Specific Antisense Oligonucleotides in SK-OV3 Cells

5,000 SK-OV3 cells/well were seeded in 96-well plates and treated with the respective ANGPTL4 antisense oligonucleotides (A24071Hi (SEQ ID NO.54), A24022Hi (SEQ ID NO.25), A24076He (SEQ ID NO.47), A24044He (SEQ ID NO.47), A24023Hi (SEQ ID NO.26), A24077He (SEQ ID NO.47), A24075He (SEQ ID NO.5), A24073Hi (SEQ ID NO.55)) at a final concentration of 10 μM. To induce ANGPTL4 mRNA expression, cells were simultaneously treated with 1 μM PPARδ. (Sigma Aldrich, cat. no. SML1491; for preparation of 10 mM stock solution see example 1). For final concentration of 1 μM PPARδ, cells seeded in 96-well plates were incubated with 100 μM medium supplemented with 0.01 μM PPARδ stock solution. As a negative control, cells were treated with equal volume of DMSO.

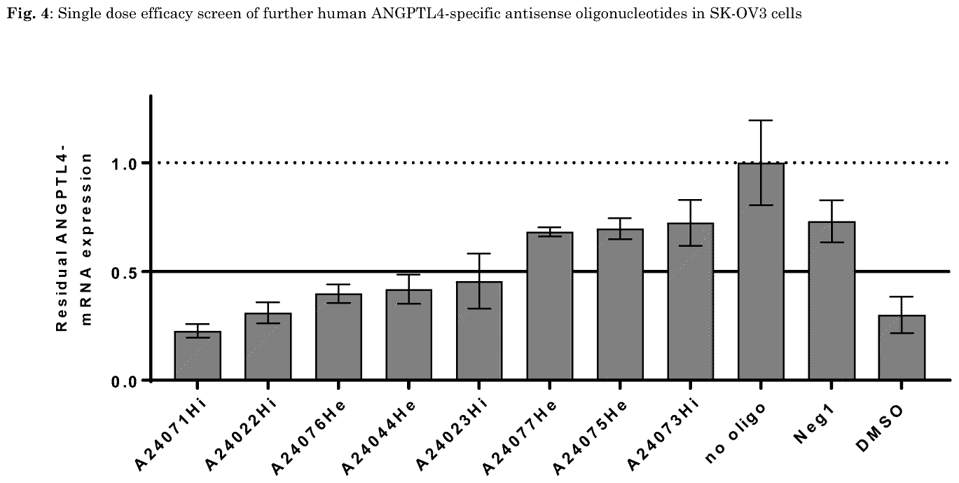

Three days after start of treatment, cells were lysed and human HPRT1 as well as human ANGPTL4 mRNA expression was measured using the QuantiGene RNA Singleplex assay. ANGPTL4-mRNA expression values were normalized to expression of the housekeeping gene HPRT1. Residual ANGPTL4-mRNA expression relative to mock-treated cells (“no oligo” set as 1) is shown in FIG. 4. Solid line and dotted lines indicate 50% and 0% knockdown efficacy, respectively. Data are represented as mean of triplicate wells+/−SD.

Repetition of the optimized ANGPTL4-specific antisense oligonucleotide screening in SK-OV3 cells resulted in five ANGPTL4 antisense oligonucleotides (A24071Hi (SEQ ID NO.54), A24022Hi (SEQ ID NO.25), A24076He (SEQ ID NO.47), A24044He (SEQ ID NO.47), A24023Hi (SEQ ID NO.26)) with more than 50% knockdown efficiency (FIG. 4).

### Example 5: In Vitro TLR9 Assay of Selected Human ANGPTL4-Specific Antisense Oligonucleotides

Binding of immune stimulatory ligands, e.g. bacterial DNA or immune stimulatory oligonucleotides with or without unmethylated CpG dinucleotides results in TLR activation. As immune activation can lead to severe, possibly life threatening condition of excessive cytokine release, there is an urgent need for a preclinical test system that predicts cytokine release in humans.

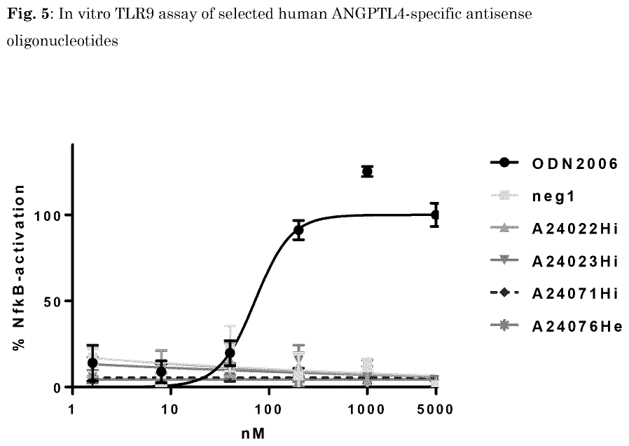

HEK-Blue-hTLR9 (Invivogen cat. no. hkb-htlr9) cells were seeded in flat-bottom 96-well plates and treated with ANGPTL4 oligonucleotides A24022Hi (SEQ ID NO. 25), A24023Hi (SEQ ID NO. 26), A24071Hi (SEQ ID NO.54) and A24076He (SEQ ID NO.47) for 24 h. Then, cell supernatants were harvested and incubated for 4 h with QUANTI-Blue solution (Invivogen cat. no. rep-qbs). SEAP activity was determined by measurement of the optical density. Means and standard deviations of OD units relative to OD units from cells stimulated with 5000 nM ODN2006 (set as 100) are depicted in FIG. 5. Data are represented as mean of triplicate wells+/−SD.

As shown in FIG. 5, none of the tested ANGPTL4-specific antisense oligonucleotides induced TLR9 activation. In contrast, positive control CpG oligonucleotide ODN2006 (5′-TCGTCGTTTTGTCGTTTTGTCGTT-3′ PTO-modified-Invivogen cat. no. tlrl-2006; SEQ ID NO. 153) clearly stimulated NFκB activation (FIG. 5).

### Example 6: IC₅₀ Determination of Selected Human ANGPTL4-Specific Antisense Oligonucleotides

30,000 primary human hepatocytes/well were seeded in 96-well plates and treated with different ANGPTL4 antisense oligonucleotides in different concentrations of 5000 nM, 1000 nM, 200 nM, 40 nM, 8 nM and 1.6 nM: A24022Hi (SEQ ID NO.25), A24023Hi (SEQ ID NO.26), A24071Hi (SEQ ID NO.54) and A24076He (SEQ ID NO.47). To induce ANGPTL4 mRNA expression, cells were simultaneously treated with 1 μM PPARγ. (Sigma Aldrich, cat. no. R2408). PPARγ stock solution (10 mM) was prepared by dissolving 10 mg PPARγ (Molecular weight: 357.43) in 2.8 ml DMSO. For final concentration of 1 μM PPARγ, cells seeded in 96-well plates were incubated with 100 μl medium supplemented with 0.01 μM PPARγ stock solution. As a negative control, cells were treated with equal volume of DMSO. Every 24 h, 70 μl of supernatant was replaced with fresh medium containing 1 μM PPARγ as well as the respective ANGPTL4 antisense oligonucleotides at indicated concentrations. Three days after start of treatment, cells were lysed and HPRT1 and ANGPTL4 mRNA expression was measured using the QuantiGene RNA Singleplex assay. ANGPTL4-mRNA expression values were normalized to expression of the housekeeping gene HPRT1. Residual ANGPTL4-mRNA expression relative to mock-treated cells (set as 1). For graphic representation mock-treated cells were set as 0.32 nM. Data are represented as mean of triplicate wells+/−SD.

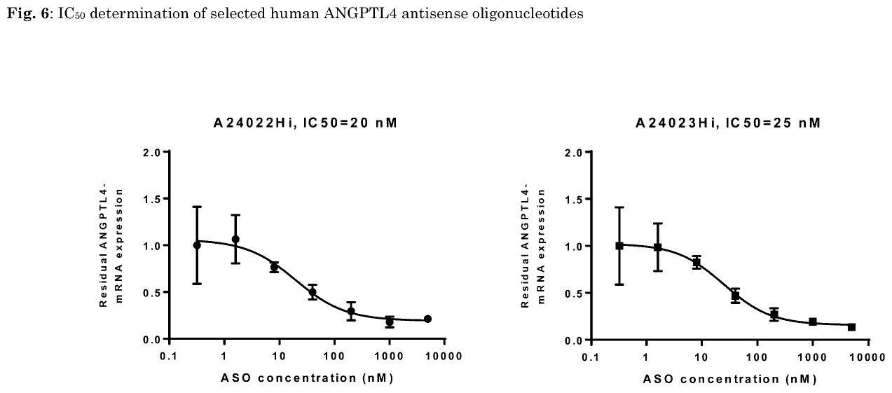

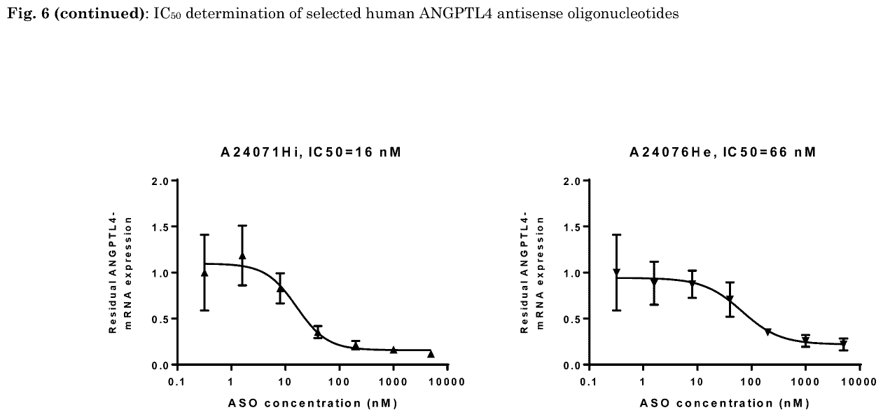

FIG. 6 and Table 5 demonstrate that the selected ANGPTL4-specific antisense oligonucleotides inhibit ANGPTL4 mRNA expression dose-dependently with IC₅₀ values in the nanomolar range.

TABLE 5
IC₅₀ values of selected human
ANGPTL4-specific antisense
oligonucleotides determined in
primary human hepatocytes.

| | IC50 | |
|---|---|---|
| ASO | [nM] | R square |
| A24022Hi | 20 | 0.82 |
| A24023Hi | 25 | 0.83 |
| A24071Hi | 16 | 0.83 |
| A24076He | 66 | 0.76 |

### Example 7: 1^{st} Single-Dose Screens of Additional Human ANGPTL4-Specific Antisense Oligonucleotides (ASOs) in Primary Hepatocytes

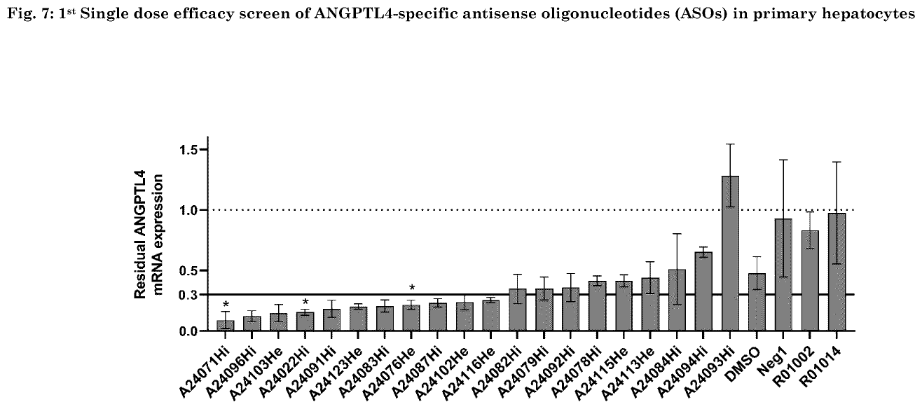

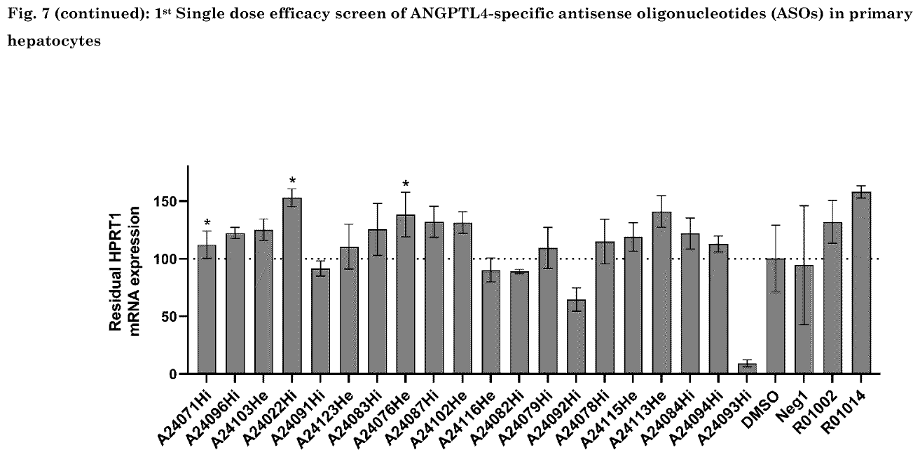

In total further 49 ANGPTL4-specific antisense-oligonucleotides were designed. Based on two initial screens in human cell lines (data not shown), 17 promising ASOs were selected for first screen in primary human hepatocytes (FIG. 7). Three control oligonucleotide (R01002, R01014, Neg1) with different lengths (16, 17 and 18 nucleotides, respectively) that do not have sequence complementarity to any human or mouse mRNA were included as negative controls, while three ANGPTL4-specific oligonucleotides with verified knockdown efficiency were used as positive controls (A24022Hi (SEQ ID NO.25), A24071Hi (SEQ ID NO.54), A24076He (SEQ ID NO.47)). Human primary hepatocytes (Lonza) were treated with the respective oligonucleotides at a single concentration of 5 μM for three days without the use of a transfection reagent. To induce ANGPTL4 mRNA expression, cells were simultaneously treated with 1 μM PPARγ (Sigma Aldrich, cat. no. R2408; for preparation of 10 mM stock solution see example 6). For final concentration of 1 μM PPARγ, cells seeded in 96-well plates were incubated with 100 μl medium supplemented with 0.01 μl PPARγ stock solution. As a negative control, cells were treated with equal volume of DMSO.

Three days after start of treatment, cells were lysed and mRNA levels were determined by QuantiGene RNA Singleplex assay. Hypoxanthine phosphoribosyltransferase 1 (HPRT1) was used as a housekeeping gene for normalization of ANGPTL4 expression.

As shown in FIG. 7, PPARγ stimulation resulted in ˜50% increased ANGPTL4 levels (no oligo) compared to DMSO-treated cells.

Treatment with eight ANGPTL4-specific ASOs (A24096Hi (SEQ ID NO.172), A24103He (SEQ ID NO.179), A24091Hi (SEQ ID NO.167), A24123He (SEQ ID NO.197), A24083Hi (SEQ ID NO.159), A24087Hi (SEQ ID NO.163), A24102He (SEQ ID NO.178), A24116He (SEQ ID NO.192)) as well as with the positive control ASOs (A24022Hi (SEQ ID NO.25), A24071Hi (SEQ ID NO.54), A24076He (SEQ ID NO.47)) reduced ANGPTL4 expression by more than 70% (equivalent to residual ANGPTL4-mRNA expression of <0.3). Control oligonucleotides (Neg1, R01002, R01014) did not reduce ANGPTL4 mRNA expression.

### Example 8: 2^{nd} Single-Dose Screens of Additional Human ANGPTL4-Specific Antisense Oligonucleotides (ASOs) in Primary Hepatocytes

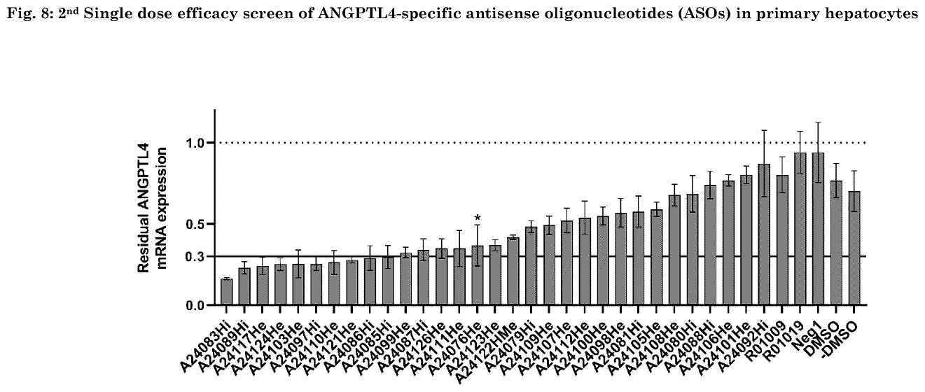

Remaining newly designed ANGPTL4-specific ASOs were tested in primary hepatocytes under the experimental conditions of Example 7 (FIG. 8). These ASOs were well tolerated in a subsequent in vitro test (data not shown). One ANGPTL4-specific oligonucleotide with verified knockdown efficiency was used as positive control (A24076He (SEQ ID NO.47)), while three control oligonucleotides (R01009, R01019, Neg1) with different lengths (16, 17 and 18 nucleotides, respectively), that do not have sequence complementarity to any human or mouse mRNA were included as negative controls.

Treatment with ten ANGPTL4-specific ASOs (A24083Hi (SEQ ID NO.159), A24089Hi (SEQ ID NO.165), A24117He (SEQ ID NO.193), A24124He (SEQ ID NO.46), A24103He (SEQ ID NO.179), A24097Hi (SEQ ID NO.173), A24110He (SEQ ID NO.186), A24121He (SEQ ID NO.195), A24086Hi (SEQ ID NO.162), A24085Hi (SEQ ID NO.161)) reduced ANGPTL4 expression by more than 70% (equivalent to residual ANGPTL4-mRNA expression of <0.3), while incubation with positive control ASO A24076H decreased ANGPTL4 mRNA expression by about 63% (FIG. 8). Control oligonucleotides (Neg1, R01009, R01019) only slightly reduced ANGPTL4 mRNA expression (by 6-20%).

### Example 9: Activation of Human Toll-Like Receptor 9 (hTLR9) in Response to Human Angiopoietin-Like Protein 4 (ANGPTL4)-Specific LNA-Modified Antisense Oligonucleotides

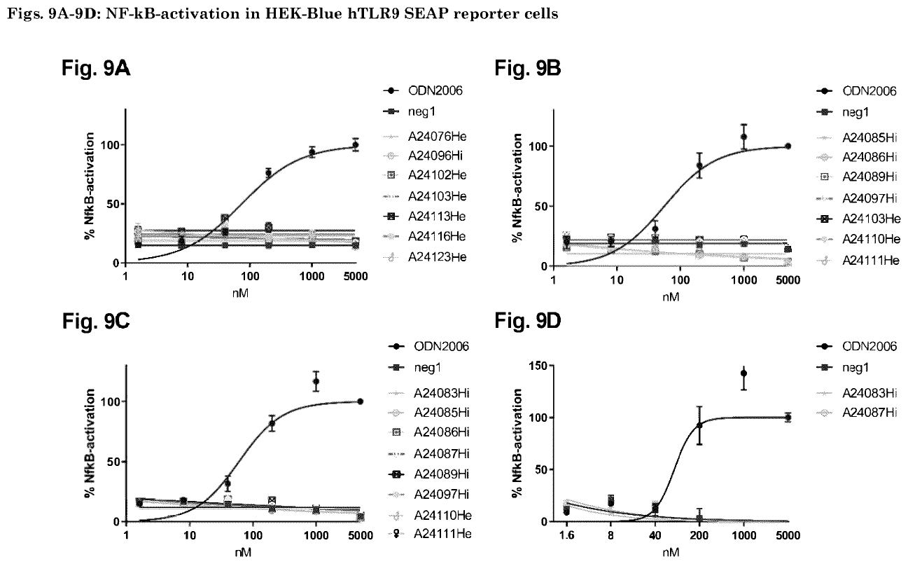

A potential of human ANGPTL4-specific LNA-modified antisense oligonucleotides A24076H (SEQ ID NO.47), A24083Hi (SEQ ID NO.159), A24085Hi (SEQ ID NO.161), A24086Hi (SEQ ID NO.162), A24087Hi (SEQ ID NO.163), A24089Hi (SEQ ID NO.165), A24096Hi (SEQ ID NO.172), A24102He (SEQ ID NO.178), A24103He (SEQ ID NO.179), A24110He (SEQ ID NO.186), A24111He (SEQ ID NO.187), A24113He (SEQ ID NO.189), A24116He (SEQ ID NO.192) and A24123He (SEQ ID NO.197) were tested to activate TLR9. The experiment was conducted once (A24076H, A24096H, A24102He, A24113He, A24116He and A24123He) or twice (all other ASOs) in a TLR9 reporter cell line HEK-Blue-hTLR9 cells (Invivogen cat. no. hkb-htlr9) under the experimental conditions of Example 5. As shown in FIGS. 9A, 9B, 9C, and 9D, no dose-dependent activation of NF-κB upon treatment of the cells with different concentrations of the respective human ANGPTL4 ASO was observed. In contrast, NF-κB was activated in a dose-dependent manner upon treatment with the positive control ODN2006 (5′-TCGTCGTTTTGTCGTTTTGTCGTT-3′ PTO-modified-Invivogen cat. no. tlrl-2006; SEQ ID NO. 153).

### Example 10: IC₅₀ Determination of Selected Human ANGPTL4-Specific Antisense Oligonucleotides (ASOs)

Based on knockdown efficiency in primary hepatocytes (FIGS. 7 and 8), ASOs with most potent knockdown efficacy and no induction of caspase3/7 upon transfection (data not shown) were selected for determination of IC₅₀ values. As a positive control ANGPTL4-specific ASO A24076He (SEQ ID NO.47) with verified knockdown efficiency was used.

Primary human hepatocytes (Primacyt) were treated with ANGPTL4-specific ASOs or negative control oligonucleotides Neg1, R01009 and R01019 at different concentrations for three days. Simultaneously, cells were treated with PPARγ (1 μM) (Sigma Aldrich, cat. no. R2408; for preparation of 10 mM stock solution see example 6) to induce ANGPTL4 expression. For final concentration of 1 μM PPARγ, cells seeded in 96-well plates were incubated with 100 μl medium supplemented with 0.01 μl PPARγ stock solution. As a negative control, cells were treated with equal volume of DMSO. After three days, mRNA expression was analyzed using the QuantiGene Singleplex RNA assay.

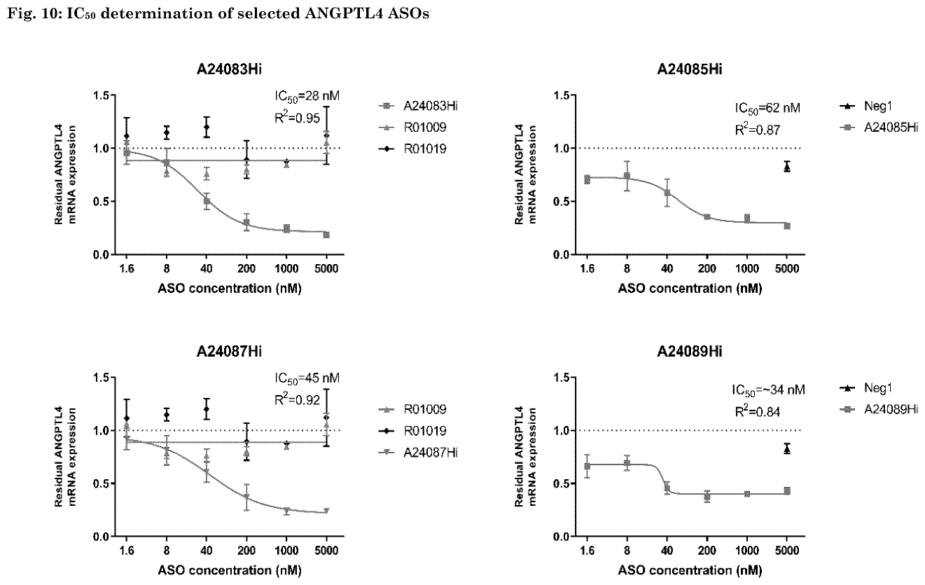

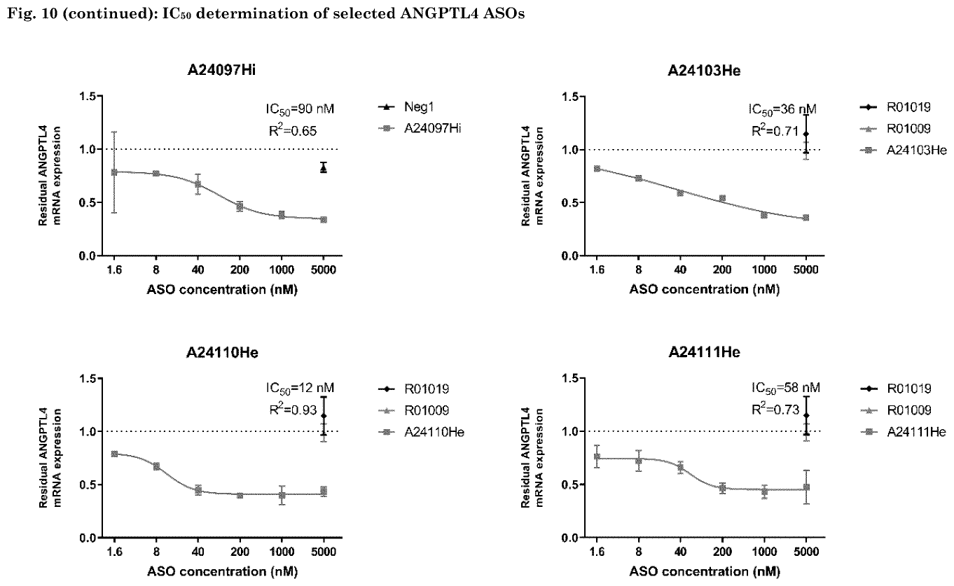

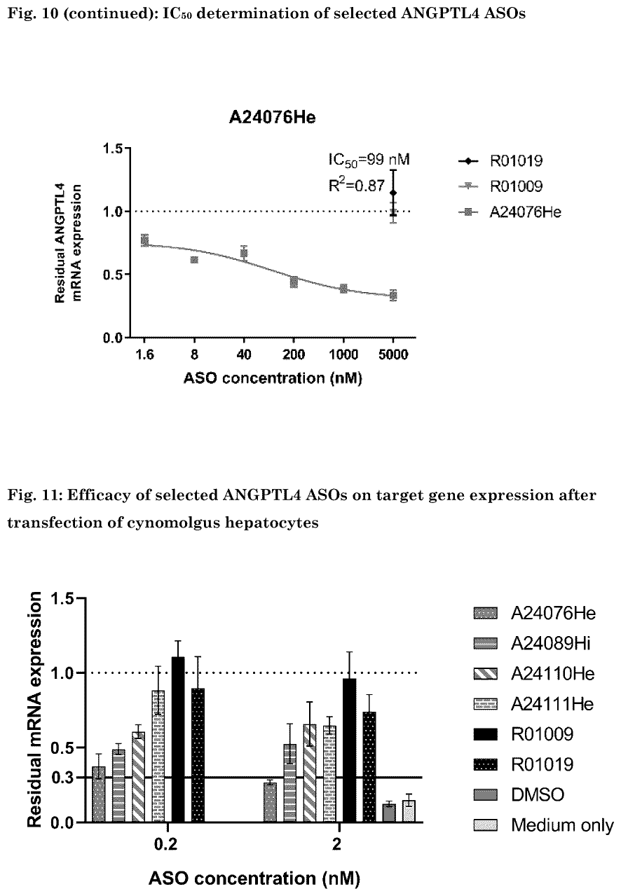

Table 6 and FIG. 10 demonstrate that the eight ANGPTL4-specific ASOs (A24083Hi (SEQ ID NO.159), A24085Hi (SEQ ID NO.161), A24087Hi (SEQ ID NO.163), A24089Hi (SEQ ID NO.165), A24097Hi (SEQ ID NO.173), A24103He (SEQ ID NO.179), A24110He (SEQ ID NO.186) and A24111He (SEQ ID NO.187)) and the positive control A24076He (SEQ ID NO.47) inhibit ANGPTL4 mRNA expression dose-dependently with IC₅₀ values in the nanomolar range. Treatment with ASO A24086Hi (SEQ ID NO.162) did not result in dose-dependent reduction of ANGPTL4 mRNA expression (R squared=0.5). Therefore, data were not included.

Following Table 6 shows IC₅₀ values and R squares of selected human ANGPTL4-specific antisense oligonucleotides determined in primary human hepatocytes. *, R square below 0.85.

| | IC₅₀ | |
|---|---|---|
| ASO | [nM] | R square |
| A24083Hi | 28 | 0.95 |
| A24085Hi | 62 | 0.87 |
| A24087Hi | 45 | 0.92 |
| A24089Hi | ~34 | 0.84* |
| A24097Hi | 90 | 0.65* |
| A24103He | 36 | 0.71* |
| A24110He | 12 | 0.93 |
| A24111He | 58 | 0.73* |
| A24076He | 99 | 0.87 |

### Example 11: In Vitro Efficacy of Human ANGPTL4-Specific Oligonucleotides In Cynomolgus Hepatocytes

In vivo tolerated human ANGPTL4-specific antisense oligonucleotides A24076H (SEQ ID NO.47) as well as three further human ANGPTL4-specific ASOs having only 1 mismatch to cynomolgus ANGPTL4 sequence (Table 7) were tested in primary cynomolgus hepatocytes (FIG. 11). All ASOs tested were shown to have verified knockdown efficiency in human cell lines and primary hepatocytes. Two control oligonucleotides (R01009, R01019) with different lengths (16 and 17 nucleotides, respectively), that do not have sequence complementarity to any human or mouse RNA were included as negative controls.

Table 7 shows human ANGPTL4-specific ASOs with proven knock-down efficiency in human cells that do not lead to caspase 3/7 induction in vitro. Depicted are cross-reactivity (CrossReact) as well as number of mismatches to cynomolgus (Mfa, macaca fascicularis) ANGPTL4 sequence, as well as activity in primary cynomolgus hepatocytes in vitro (FIG. 11):

| | | | | Mis- | Activity |
|---|---|---|---|---|---|
| | | SEQ | | matches | in Mfa |
| | | ID | | to Mfa | hepa- |
| Target | ASO ID | NO. | CrossReact_Mfa | sequence | tocytes |
| ANGPTL4 | A24076He | 47 | yes | 0 | + |
| | A24083Hi | 159 | no | 4 | NA |
| | A24085Hi | 161 | no | 4 | NA |
| | A24086Hi | 162 | no | 6 | NA |
| | A24087Hi | 163 | no | 3 | NA |
| | A24089Hi | 165 | no | 1 | + |
| | A24097Hi | 173 | no | 3 | NA |
| | A24103He | 179 | no | 5 | NA |
| | A24110He | 186 | no | 1 | + |
| | A24111He | 187 | no | 1 | + |

Primary cynomolgus hepatocytes (Primacyt) were transfected with ANGPTL4-specific ASOs or negative control oligonucleotides R01009 and R01019 at different concentrations for three days. Simultaneously, cells were treated with PPARδ (1 μM) (Sigma Aldrich, cat. no. R2408; for preparation of 10 mM stock solution see example 6) to induce ANGPTL4 expression. For final concentration of 1 μM PPARγ, cells seeded in 96-well plates were incubated with 100 μl medium supplemented with 0.01 μl PPARγ stock solution. As a negative control, cells were treated with equal volume of DMSO. After three days, mRNA expression was analyzed using the QuantiGene Singleplex RNA assay.

As shown in FIG. 11, treatment with PPARδ induced ANGPTL4 expression about 8-fold (FIG. 11, DMSO control). Treatment with the ANGPTL4-specific ASO A24076He (SEQ ID NO.47), which is completely cross-reactive to cynomolgus ANGPTL4 sequence led to more than 70% knock-down of ANGPTL4 (equivalent to residual mRNA expression of 0.3). Human ANGPTL4-specific oligonucleotides having one mismatch to cynomolgus sequence (A24089Hi (SEQ ID NO.165), A24110He (SEQ ID NO.186), A24111He (SEQ ID NO.187)) also reduced cynomolgus ANGPTL4 mRNA up to 51% (equivalent to residual ANGPTL4-mRNA expression of 0.49). Negative control oligonucleotide R01009 did not reduce Angptl4 expression in primary cynomolgus hepatocytes, while control oligonucleotide R01019 only slightly decreased ANGPTL4 mRNA expression by about 25% (equivalent to residual ANGPTL4-mRNA expression of 0.75) using a concentration of 2 nM.

### Conclusion Based on Example 11

Taken together, the in vitro experiments led to the identification of highly potent human ANGPTL4 specific ASOs that are eligible for testing of an ASO-based ANGPTL4-targeting therapeutic in cynomolgus monkeys. Such a drug is used for example for the systemic treatment of dyslipidemia patients in order to reduce the ANGPTL4-mediated inhibition of lipoprotein lipase L preventing cellular lipid overload, obesity, diabetes type II and cardiovascular disease.

### Example 12: 1^{st} Single-Dose Screen of Mouse ANGPTL4-Specific Antisense Oligonucleotides in 3T3 Cells

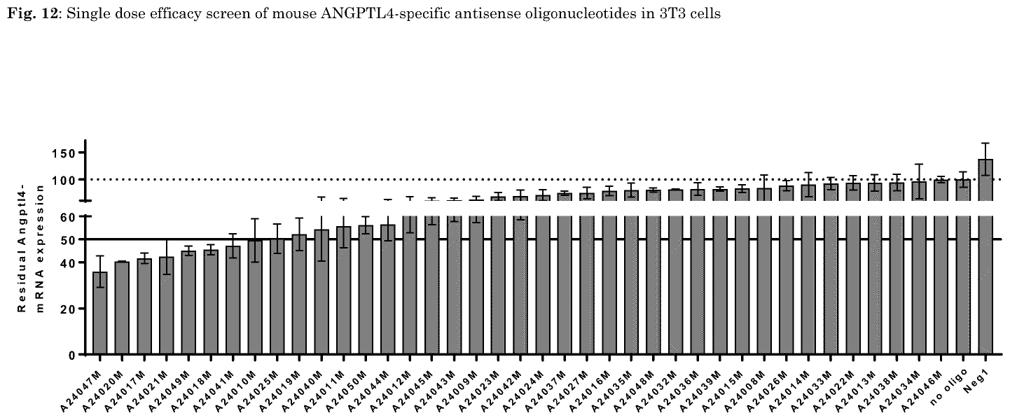

4,500 3T3 cells/well were seeded in 96-well plates and treated with the ANGPTL4 antisense oligonucleotides as shown in FIG. 12 at a final concentration of 10 μM. Cells were lysed and mouse Hprt1 and mouse ANGPTL4 mRNA expression was measured using the QuantiGene RNA Singleplex assay. ANGPTL4-mRNA expression values were normalized to expression of the housekeeping gene Hprt1. Residual ANGPLT4-mRNA expression relative to mock-treated cells (“no oligo” set as 100) is shown in FIG. 12. Solid line and dotted line indicate 50% and 0% knockdown efficacy, respectively. Data are represented as mean of triplicate wells+/−SD.

As shown in FIG. 12, eight ANGPTL4 antisense oligonucleotides, i.e., A24047M (SEQ ID NO.106), A24020M (SEQ ID NO.79), A24017M (SEQ ID NO.76), A24021M (SEQ ID NO.80), A24049M (SEQ ID NO.108), A24018M (SEQ ID NO.77), A24041M (SEQ ID NO. 100) and A24010M (SEQ ID NO.69), reduced the normalized ANGPTL4 expression by more than 50%, while control oligonucleotide (Neg1) did not reduce ANGPTL4 mRNA expression (FIG. 12).

### Example 13: 2^{nd} Single-Dose Efficacy Screen of Mouse ANGPTL4-Specific Antisense Oligonucleotides in Renca Cells

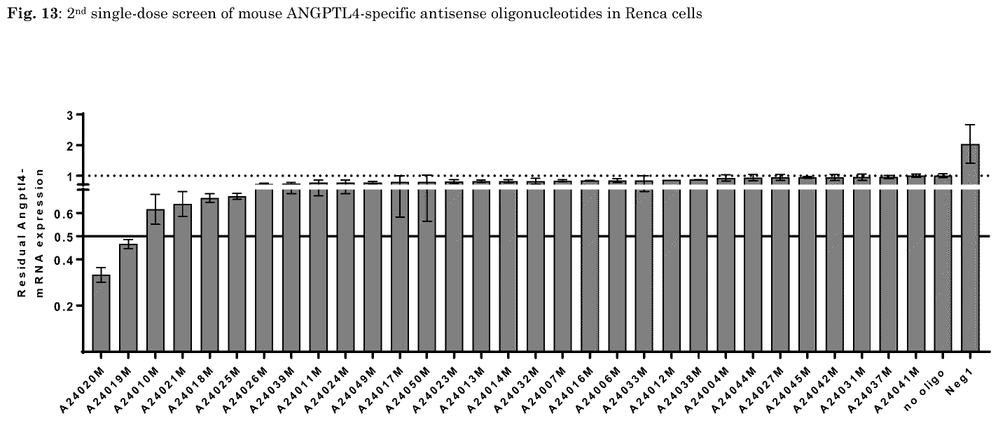

5,000 Renca cells/well were seeded in 96-well plates and treated with the ANGPTL4 antisense oligonucleotides as shown in FIG. 13 at a final concentration of 10 μM. Three days after start of treatment, cells were lysed and mouse Hprt1 and mouse ANGPTL4 mRNA expression was measured using the QuantiGene RNA Singleplex assay. ANGPTL4-mRNA expression values were normalized to expression of the housekeeping gene Hprt1. Residual ANGPTL4-mRNA expression relative to mock-treated cells (“no oligo” set as 1) is shown. Solid line and dotted line indicate 50% and 0% knockdown efficacy, respectively. Data are represented as mean of triplicate wells+/−SD. As shown in FIG. 13, treatment with two ANGPTL4-specific antisense oligonucleotides, which are A24020M (SEQ ID NO.79) and A24019M (SEQ ID NO.78) resulted in ANGPTL4 knockdown of more than 50% (equivalent to residual mRNA level of <0.5) (FIG. 13).

### Example 14: Test of Further Mouse ANGPTL4-Specific Antisense Oligonucleotides in 4T1 Cells

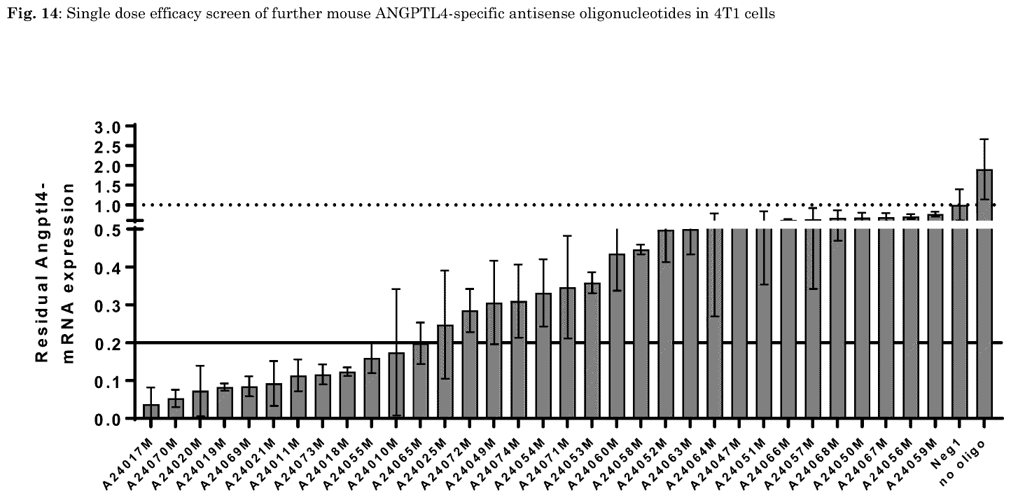

2,500 4T1 cells/well were seeded in 96-well plates and treated with the ANGPTL4 antisense oligonucleotides (ASO) as shown in FIG. 14 at a final concentration of 5 μM. After 3 d, cell supernatant was replaced by fresh medium w/ASO. Six days after start of treatment, cells were lysed and mouse Gapdh and mouse ANGPTL4 mRNA expression were measured using the QuantiGene RNA Singleplex assay. ANGPTL4-mRNA expression values were normalized to expression of the housekeeping gene Gapdh. Residual ANGPTL4-mRNA expression relative to Neg1-treated cells (set as 1) is shown. Solid line and dotted lines indicate 80% and 0% knockdown efficacy, respectively. Data are represented as mean of triplicate wells+/−SD.

As shown in FIG. 14, 12 of the tested antisense oligonucleotides, which are A24017M (SEQ ID NO.76), A24070M (SEQ ID NO.127), A24020M (SEQ ID NO.79), A24019M (SEQ ID NO.78), A24069M (SEQ ID NO.126), A24021M (SEQ ID NO.80), A24011M (SEQ ID NO.70), A24073M (SEQ ID NO.130), A24018M (SEQ ID NO.77), A24055M (SEQ ID NO.114), A24010M (SEQ ID NO.69), and A24065M (SEQ ID NO.79) show more than 80% knockdown of ANGPTL4 mRNA (equivalent to residual mRNA level of <0.2).

### Example 15: Single-Dose Efficacy Screen of Intron-Targeting Mouse ANGPTL4-Specific Antisense Oligonucleotides in 4T1 Cells

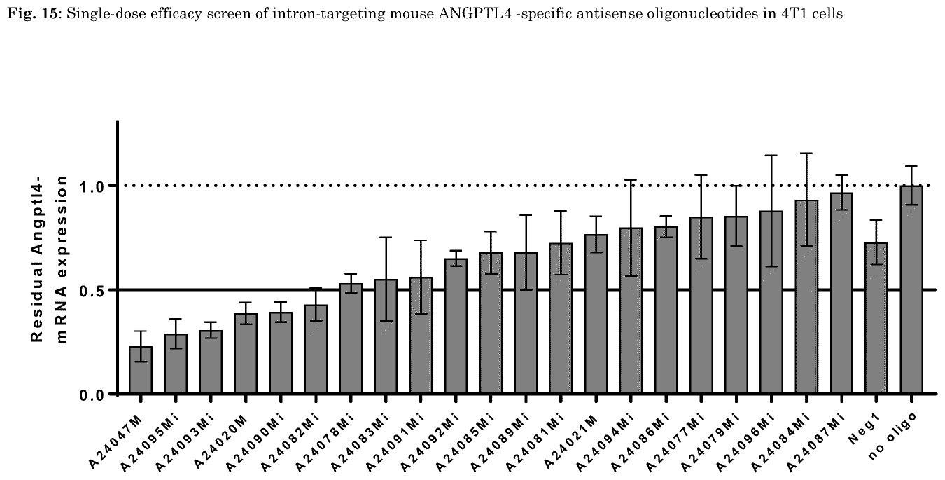

2,500 4T1 cells/well were seeded in 96-well plates and treated with the ANGPTL4 antisense oligonucleotides (ASO) as shown in FIG. 15 at a final concentration of 5 μM. Three days after start of treatment, cell supernatant was replaced by fresh medium w/ASO and cells were incubated for additional 3 d. Then, cells were lysed and mouse Hprt1 and mouse ANGPTL4 mRNA expression was measured using the QuantiGene RNA Singleplex assay. ANGPTL4-mRNA expression values were normalized to expression of the housekeeping gene Hprt1. Residual ANGPTL4-mRNA expression relative to mock-treated cells (“no oligo” control set as 1) is shown. Solid line and dotted lines indicate 50% and 0% knockdown efficacy, respectively. Data are represented as mean of triplicate wells+/−SD.

As shown in FIG. 15, six of the tested ANGPTL4 antisense oligonucleotides, which are A24047M (SEQ ID NO.106), A24095Mi (SEQ ID NO.151), A24093Mi (SEQ ID NO.149), A24020M (SEQ ID NO.79), A24090Mi (SEQ ID NO.146), and A24082Mi (SEQ ID NO.139) show more than 50% knockdown of ANGPTL4 mRNA (equivalent to residual mRNA level of <0.5).

### Example 16: IC₅₀ Determination of Selected Mouse ANGPTL4-Specific Antisense Oligonucleotides

2,500 4T1 cells/well were seeded in 96-well plates and treated with the respective ANGPTL4 antisense oligonucleotides (ASO) A24018M (SEQ ID NO.77), A24019M (SEQ ID NO.78), A24020M (SEQ ID NO.79), A24021M (SEQ ID NO.80), A24047M (SEQ ID NO.106), A24054M (SEQ ID NO.113), A24065M (SEQ ID NO.79), A24070M (SEQ ID NO.127), A24072M (SEQ ID NO.129), A24082M (SEQ ID NO.139) and A24095Mi (SEQ ID NO.151) at different concentrations of 5000 nM, 1000 nM, 200 nM, 40 nM, 8 nM and 1.6 nM. Three days after start of treatment, cell supernatant was replaced by fresh medium w/ASO and cells were incubated for additional 3 d. Then, cells were lysed and mouse Hprt1 and mouse ANGPTL4 mRNA expression was measured using the QuantiGene RNA Singleplex assay. ANGPTL4-mRNA expression values were normalized to expression of the housekeeping gene Hprt1. Residual ANGPTL4-mRNA expression relative to mock-treated cells (“no oligo” control set as 1) is shown. Data are represented as mean of triplicate wells+/−SD.

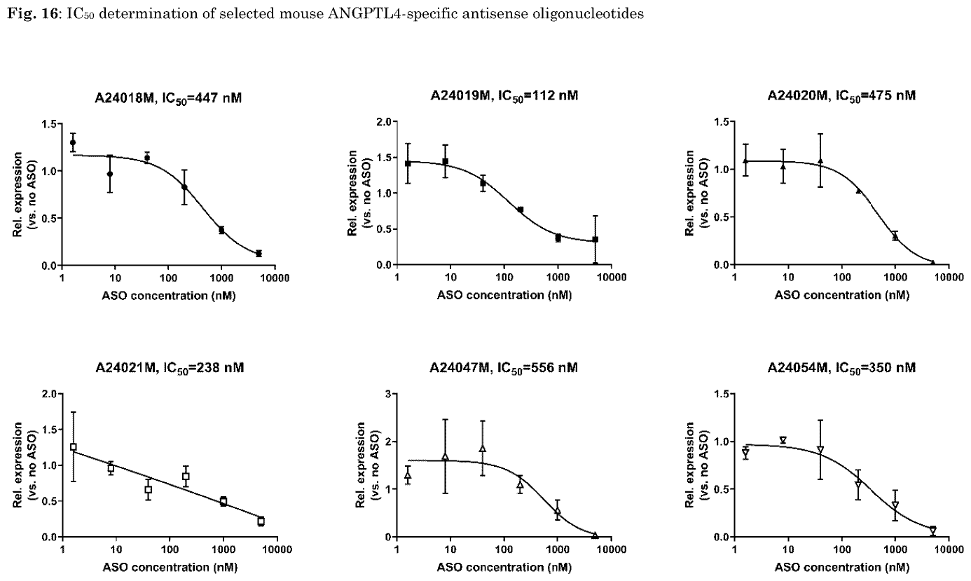

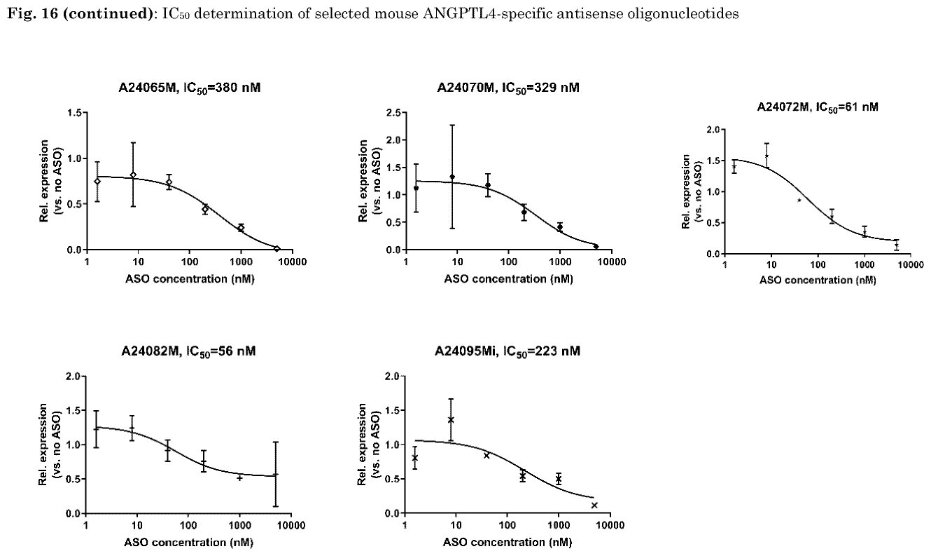

FIG. 16 and Table 8 demonstrate that the selected ANGPTL4-specific antisense oligonucleotides inhibit ANGPTL4 mRNA expression dose-dependently with IC₅₀ values in the nanomolar range.

TABLE 8
IC₅₀ values of selected mouse ANGPTL4-specific
antisense oligonucleotides determined in 4T1 cells.

| | IC₅₀ | |
|---|---|---|
| ASO | [nM] | R square |
| A24018M | 447 | 0.90 |
| A24019M | 122 | 0.87 |
| A24020M | 475 | 0.91 |
| A24021M | 283 | 0.68 |
| A24047M | 556 | 0.70 |
| A24054M | 350 | 0.85 |
| A24065M | 380 | 0.80 |
| A24070M | 329 | 0.59 |
| A24072M | 61 | 0.92 |
| A24082Mi | 56 | 0.65 |
| A24095Mi | 223 | 0.64 |

### Example 17: 1^{st} Single-Dose Screen of Additional Mouse Angptl4-Specific ASOs in 4T1 Cells

Mouse Angptl4-specific antisense-oligonucleotides were designed. In the initial screen 53 ASOs targeting Angptl4 mRNA were tested. Three control oligonucleotides (R01002, R01014, Neg1) with different lengths (16, 17 and 18 nucleotides, respectively), that do not have sequence complementarity to any human or mouse mRNA were included as negative controls, whereas three ANGPTL4-specific oligonucleotides (A24047M (SEQ ID NO.106), A24072M (SEQ ID NO.129), A24095Mi (SEQ ID NO.151)) with verified knockdown efficiency were used as positive controls. Mouse breast cancer cells (4T1 cells) were treated with the respective oligonucleotides at a single concentration of 5 μM. After three days, cell supernatant was replaced by fresh medium containing 5 μM of the respective ASO and incubated for further three days. Afterwards, cells were lysed and mRNA levels were determined by QuantiGene RNA Singleplex assay. Hprt1 was used as a housekeeping gene for normalization of Angptl4 expression.

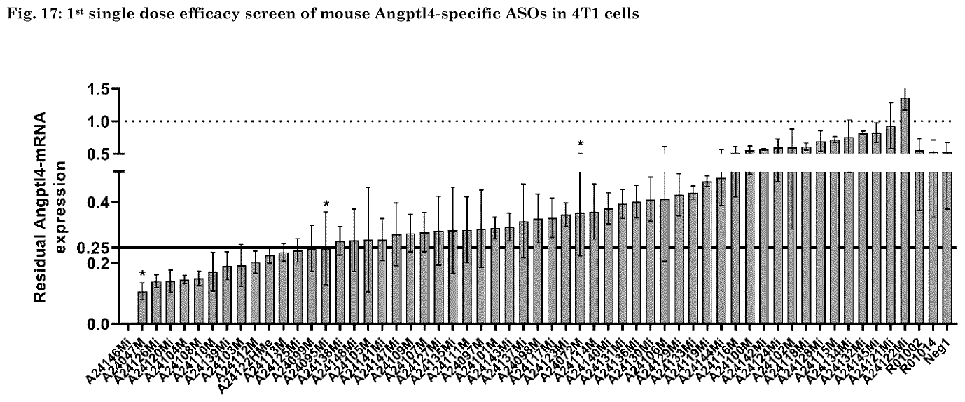

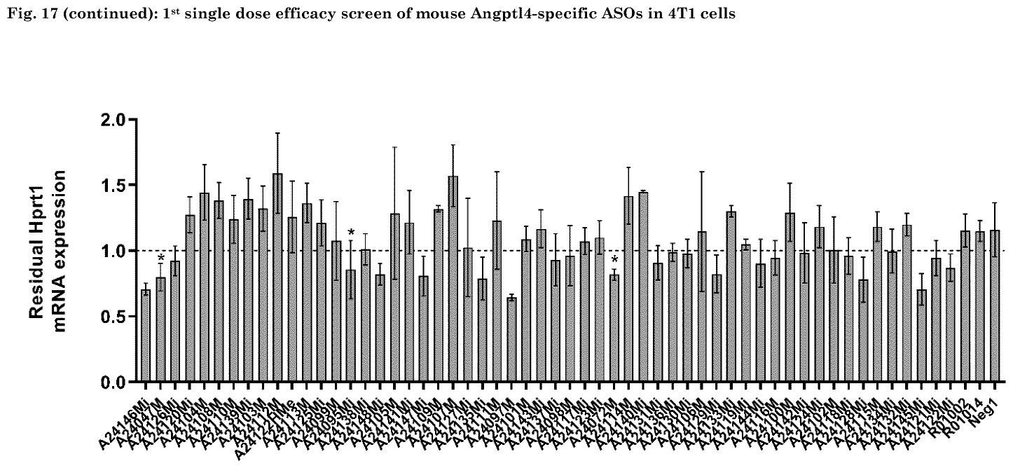

As shown in FIG. 17, 15 ASOs (A24146Mi (SEQ ID NO.247), A24047M (SEQ ID NO.106), A24126Mi (SEQ ID NO.227), A24120Mi (SEQ ID NO.221), A24104M (SEQ ID NO.207), A24108M (SEQ ID NO.211), A24110M (SEQ ID NO.213), A24139Mi (SEQ ID NO.240), A24103M (SEQ ID NO.206), A24112M (SEQ ID NO.107), A24122HMe (SEQ ID NO.196), A24113M (SEQ ID NO.108), A24125Mi (SEQ ID NO.226), A24099M (SEQ ID NO.202), A24095Mi (SEQ ID NO.151)) reduced Angptl4 expression by more than 75% (equivalent to residual Angptl4-mRNA expression of <0.25), while control oligonucleotides (Neg1, R01002, R01014) reduced Angptl4 mRNA expression by less than 50% (equivalent to residual Angptl4-mRNA expression of >0.5) (FIG. 17).

### Example 18: 2^{nd} Single-Dose Screen of Mouse Angptl4-Specific ASOs in Renca Cells

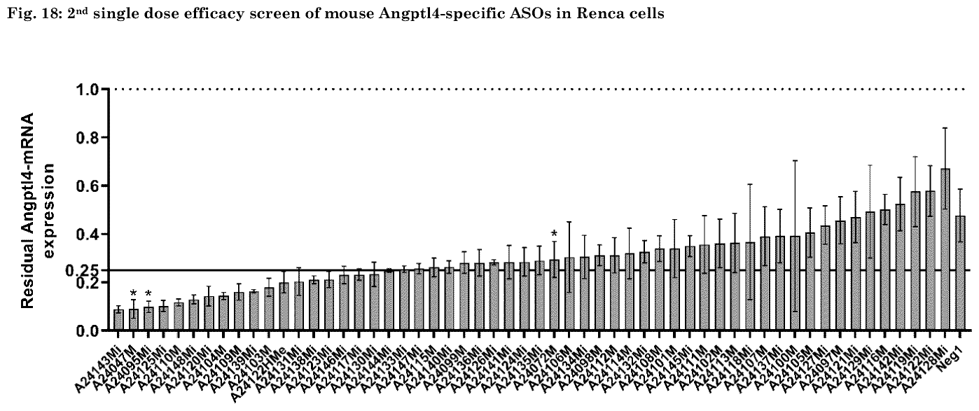

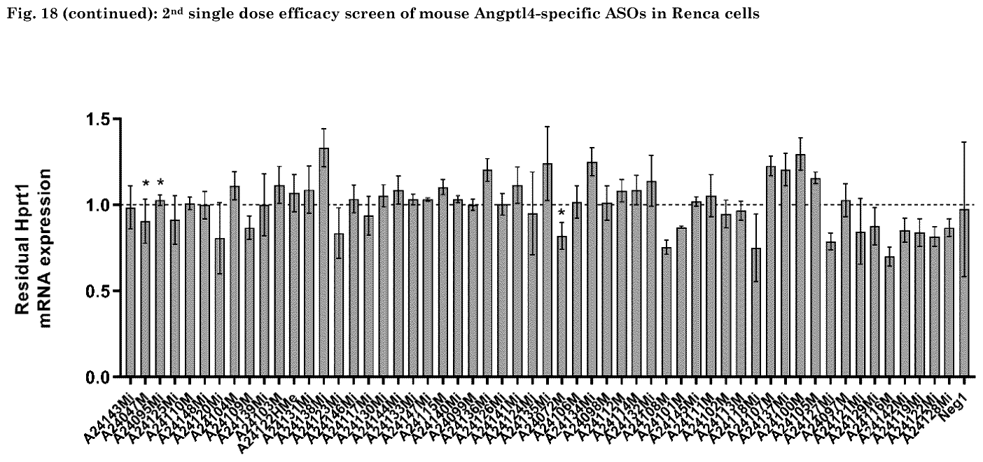

For further confirmation in a different cell line, the same treatment as in Example 17 was applied to mouse Renca cells. Thereby, treatment with 19 Angptl4-specific ASOs (A24143Mi (SEQ ID NO.244), A24047M (SEQ ID NO.106), A24095Mi (SEQ ID NO.151), A24125Mi (SEQ ID NO.226), A24110M (SEQ ID NO.213), A24148Mi (SEQ ID NO.151), A24120Mi (SEQ ID NO.221), A24104M (SEQ ID NO.207), A24109M (SEQ ID NO.212), A24139Mi (SEQ ID NO.240), A24103M (SEQ ID NO.206), A24122HMe (SEQ ID NO.196), A24131Mi (SEQ ID NO.232), A24138Mi (SEQ ID NO.239), A24123Mi (SEQ ID NO.224), A24146Mi (SEQ ID NO.247), A24117Mi (SEQ ID NO.218), A24130Mi (SEQ ID NO.231), A24144Mi (SEQ ID NO.245)) resulted in Angptl4 knockdown of more than 75% (equivalent to residual Angptl4-mRNA expression of <0.25) (FIG. 18).

### Example 19: IC₅₀ Determination of Selected Mouse ANGPTL4-Specific Antisense Oligonucleotides

Based on results of knockdown efficiencies in 4Tl and Renca cells (FIG. 17, 18), nine ASOs (A24103M (SEQ ID NO.206), A24110M (SEQ ID NO.213), A24122HMe (SEQ ID NO.196), A24120Mi (SEQ ID NO.221), A24125Mi (SEQ ID NO.226), A24139Mi (SEQ ID NO.240), A24143Mi (SEQ ID NO.244), A24146Mi (SEQ ID NO.247), A24148Mi (SEQ ID NO.151)) with most potent knockdown efficacy Renca and 4T1 cells were selected for determination of half maximal inhibitory concentration (IC₅₀) values.

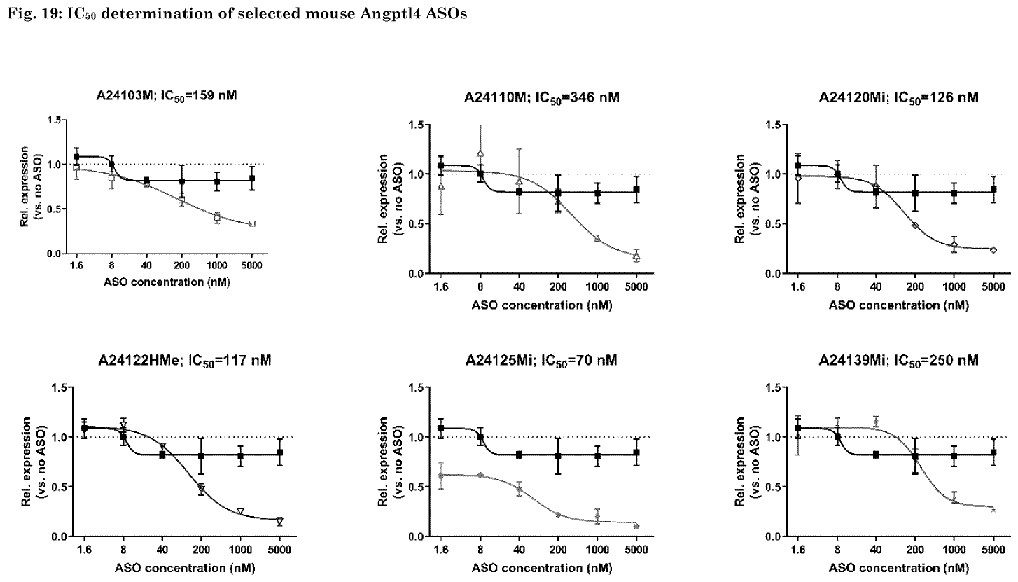

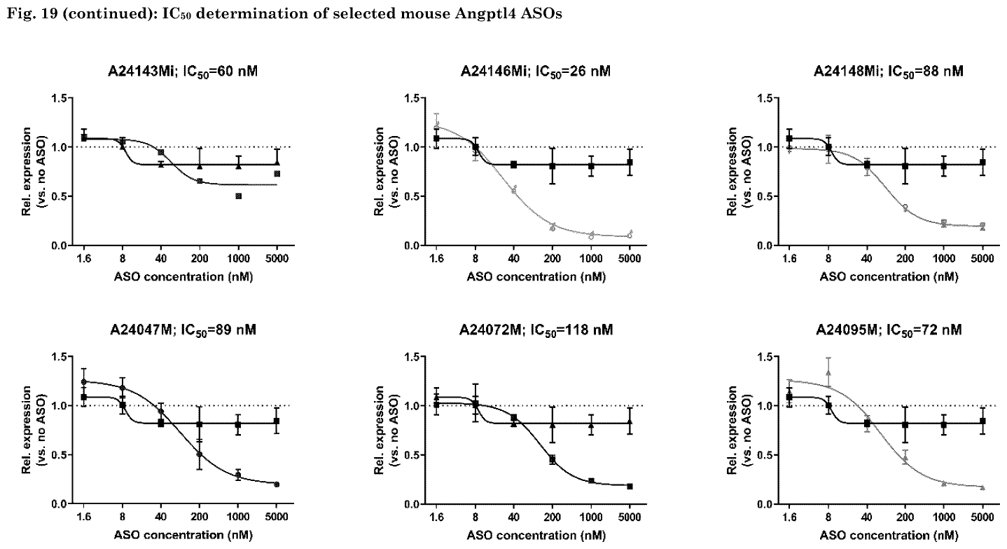

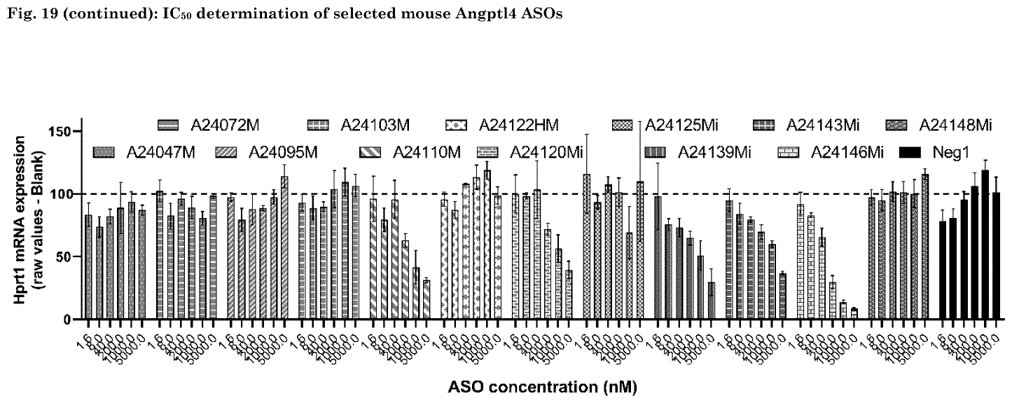

Primary mouse hepatocytes were treated with the respective ASO at different concentrations for three days. After three days, mRNA expression was analyzed using the QuantiGene Singleplex RNA assay. FIG. 19 and Table 9 demonstrate that the selected Angptl4-specific ASOs inhibit Angptl4 mRNA expression dose-dependently with IC₅₀ values in the nanomolar range.

Table 9 shows IC₅₀ values of selected Angptl4-specific ASOs determined in primary human hepatocytes. *, R square below 0.85; ASOs with increased potential to induce caspase3/7 leading to dose-dependent decrease of Hprt1 levels:

| | IC₅₀ | |
|---|---|---|
| ASO | [nM] | R square |
| A24103M | 159 | 0.90 |
| A24110M | 346 | 0.72* |
| A24122HMe | 117 | 0.98 |
| A24120Mi | 126 | 0.86 |
| A24125Mi | 70 | 0.91 |
| A24139Mi | 250 | 0.92 |
| A24143Mi | 60 | 0.84* |
| A24146Mi | 26 | 0.98 |
| A24148Mi | 88 | 0.97 |
| A24047M | 89 | 0.96 |
| A24072M | 118 | 0.96 |
| A24095Mi | 72 | 0.94 |

Conclusion Based on the Examples 7-11and 17-19

Testing a set of 47 further ASOs with specificity for human ANGPTL4, several ASOs were selected that potently reduce the expression of human ANGPTL4 in primary hepatocytes on the mRNA level. Treatment with 16 of the tested ASOs showed more than 70% knockdown of ANGPTL4 mRNA in primary human hepatocytes cells. Thereby, IC₅₀ values of most potent candidates were in low nanomolar range.

In order to conduct in vivo experiments in mouse models ASOs were designed with specificity for mouse Angptl4 and successful candidate ASOs were selected for in vivo studies that potently knock down mouse Angptl4 expression in vitro.

Taken together, a comprehensive set of in vitro experiments was conducted that led to the identification of highly potent human ANGPTL4 specific ASOs that are eligible for the development of an ASO-based ANGPTL4-targeting therapeutic.

What is claimed is:

1. A method of reducing ANGPTL4 expression in a subject, the method comprising administering a pharmaceutically active amount of an ANGPTL4 inhibitor consisting of an antisense oligonucleotide of 12 to 22 nucleotides, wherein at least one nucleotide in the antisense oligonucleotide is modified, and wherein the antisense oligonucleotide is complementary to a corresponding portion of 12 to 22 consecutive nucleotides of positions 1732-1759 of SEQ ID NO: 1 to the subject in need of the reducing, and wherein the antisense oligonucleotide inhibits the expression of ANGPTL4.

2. The method according to claim 1, wherein the antisense oligonucleotide of 12 to 22 nucleotides is a gapmer and comprises a central core of at least 5 consecutive deoxyribonucleotides and/or ribonucleotides.

3. The method according to claim 1, wherein at least one of the modified nucleotides of the antisense oligonucleotide is a 2′modified nucleotide, or a bridged nucleotide.

4. The method according to claim 3, wherein the bridged nucleotide is a 2′-4′ bridged nucleotide.

5. The method according to claim 1, wherein at least one of the modified nucleotides of the antisense oligonucleotide is selected from a locked nucleic acid (LNA), cET, ENA, a 2′Fluoro modified nucleotide, a 2′O-Methyl modified nucleotide, 2′-Methoxyethyl modified nucleotide or a combination thereof.

6. The method according to claim 1, wherein at least one of the modified nucleotides of the antisense oligonucleotide is LNA.

7. The method according to claim 1, wherein the antisense oligonucleotide comprises a modified phosphate backbone.

8. The method according to claim 7, wherein the modified phosphate is a phosphorothioate.

9. The method according to claim 1, wherein the subject has a cardiometabolic disease selected from the group consisting of obesity, diabetes, hypercholesterolemia, hypertriglyceridemia (HTG), dyslipidemia, pancreatitis, metabolic syndrome, and familial chylomicronemia syndrome (FCS).

10. The method according to claim 9, wherein the hypercholesterolemia is selected from the group consisting of homozygous familial hypercholesterolemia (HoFH) and heterozygous familial hypercholesterolemia (HeFH).

11. The method according to claim 9, wherein the diabetes is type 2 diabetes.

12. The method according to claim 1, wherein the subject has a respiratory infection.

13. The method according to claim 12, wherein the respiratory infection is influenza.

14. The method according to claim 1, wherein the subject has cancer.

15. The method according to claim 14, wherein the cancer is selected from the group consisting of breast cancer, lung cancer, malignant melanoma, lymphoma, skin cancer, bone cancer, prostate cancer, liver cancer, brain cancer, cancer of the larynx, gall bladder, pancreas, testicular, rectum, parathyroid, thyroid, adrenal, neural tissue, head and neck, colon, stomach, bronchi, kidneys, basal cell carcinoma, squamous cell carcinoma, metastatic skin carcinoma, osteo sarcoma, Ewing's sarcoma, reticulum cell sarcoma, liposarcoma, myeloma, giant cell tumor, small-cell lung tumor, islet cell tumor, primary brain tumor, meningioma, acute and chronic lymphocytic and granulocytic tumors, acute and chronic myeloid leukemia, hairy-cell tumor, adenoma, hyperplasia, medullary carcinoma, intestinal ganglioneuromas, Wilm's tumor, seminoma, ovarian tumor, leiomyomater tumor, cervical dysplasia, retinoblastoma, soft tissue sarcoma, malignant carcinoid, topical skin lesion, rhabdomyosarcoma, Kaposi's sarcoma, osteogenic sarcoma, malignant hypercalcemia, renal cell tumor, polycythermia vera, adenocarcinoma, anaplastic astrocytoma, glioblastoma multiforma, leukemia, and epidermoid carcinoma.

16. The method according to claim 15, wherein the cancer is lung cancer.

17. The method according to claim 1, wherein the oligonucleotide comprises a sequence selected from the group consisting of
(SEQ ID NO:47)
(5′-3′) CAATGTGACTGAGTCCG,
(SEQ ID NO:46)
(5′-3′) ATGTGACTGAGTCCGCC,
(SEQ ID NO:196)
(5′-3′) TGACTGAGTCCGCCATT, and
(SEQ ID NO:197)
(5′-3′) GTGACTGAGTCCGCCA.

18. The method according to claim 17, wherein the oligonucleotide comprises a sequence selected from the group consisting of
(SEQ ID NO:47)
(5′-3′) +C*+A*+A*T*G*T*G*A*C*T*G*A*G*T*+C*+C*+G,
(SEQ ID NO:47)
(5′-3′) +C*+A*+A*+T*G*T*G*A*C*T*G*A*G*T*+C*+C*+G,
(SEQ ID NO:47)
(5′-3′) +C*+A*+A*T*G*T*G*A*C*T*G*A*G*+T*+C*+C*+G,
(SEQ ID NO:46)
(5′-3′) +A*+T*+G*T*G*A*C*T*G*A*G*T*C*C*+G*+C*+C
(SEQ ID NO:196)
(5′-3′) +T*+G*+A*C*T*G*A*G*T*C*C*G*C*C*+A*+T*+T,
and
(SEQ ID NO:197)
(5′-3′) +T*+G*+T*G*A*C*T*G*A*G*T*C*C*G*+C*+C*+A,
wherein + indicates a locked nucleic acid (LNA) nucleotide and * indicates a phosphorothioate (PTO) linkage between the nucleotides.


SEQUENCE LISTING (252 sequences)

SEQ ID NO: 1 - DNA, 1905 bp, Homo sapiens
  source 1..1905: mol_type = other DNA
ataaaaaccgtcctcgggcgcggcggggagaagccgagctgagcggatcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgtcagggaaccgaggggtccaccgacctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccgagatggcccagccagttgacccggctcacaatgtcagccgcctgcaccggctgcccagggattgccaggagctgttccaggttggggagaggcagagtggactatttgaaatccagcctcaggggtctccgccatttttggtgaactgcaagatgacctcagatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcctagcgtcctggctgggcctggtcccaggcccacgaaagacggtgactcttggctctgcccgaggatgtggccgttccctgcctgggcaggggctccaaggaggggccatctggaaacttgtggacagagaagaagaccacgactggagaagccccctttctgagtgcaggggggctgcatgcgttgcctcctgagatcgaggctgcaggatatgctcagactctagaggcgtggaccaaggggcatggagcttcactccttgctggccagggagttggggactcagagggaccacttggggccagccagactggcctcaatggcggactcagtcacattgactgacggggaccagggcttgtgtgggtcgagagcgccctcatggtgctggtgctgttgtgtgtaggtcccctggggacacaagcaggcgccaatggtatctgggcggagctcacagagttcttggaataaaagcaacctcagaacactttg

SEQ ID NO: 2 - DNA, 10223 bp, Homo sapiens
  source 1..10223: mol_type = other DNA
ggggagaagccgagctgagcggatcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgtcagggaaccgaggggtccaccgacctcccgttagcccctgagagccgggtggaccctgaggtccttcacagcctgcaggtacgtgtccccagggctggttctccgcgcccctagtggctctcctggcttggaagggtatggacaggagtggggcgtgggggcggggtgcgcaactgtggctccctgggcttccctgcgtcaagggatgggctccccccttaggaagccgaggagggaggttcgctcaaggccaggaattcaagaccacctaaagcaaaatagcgagacccccgtctcttctacacacacacacacacacacacaaaataaataaaataaaataaaataaatataaaattaaaaaacgaccgggcgcagtggctcacggagcctccacctgctgtcgagactagcctggccaacatggagaaacctcgtctctactaaaaatacaaaaaattagcctggcatggtgtgcatgcctgtaatcccagctacttgggaggctgaggcaggagaatctcttgaacccgggaggcagaggttgcagtgagccgagatcgcagcactgcactccagcctgggcaacagagtgagactcctcaaaaaaataatagcgataaaataaaaataaagccaggtgcggtgactcacgcttgtagtcccagcactttgggagggcgaggccagtggatcacctgaggtcaggagttcaagaccagcctgaccaacacagtgaaaccccgtctctactaaaaatacaaaaattagctgggtgtggtggtgggcgcctgtaatcccagctactggggaggctgaggcaggagaacctcttgaacccgggaggcggaggttgcagtgagccgggatcatgccattgcactccagcctgggcgacagagctcgactccatctcaaaataataataataataaataaaataaaatacaaatacaaaaattagccaggtgtggtggcagctacttgggaggctgagacacaagaatcacttgagtctgggaggcagaggttgcagtgagccaagattgcgccactgcactccaacctgggtgacagagcgagactctgtctgaaaaaataaaaaataagccaggcatagagctgcatgactgtagtcccagctacacaggaggctgaaaacaggaggatcgcctgaccccaggagttggaggctgcagtgagctgtgacagcaccactgcactccagcctgggcgagagaacgagaccccgtcattgggaaaaaaaagaaaaaagaaagactcccttgcctggcctcagcggatggagatttggaaggatggatgagtggatgaattaagaggttggggtaggcaagctgggtcctcaccaaggttttcacccctccccagacacaactcaaggctcagaacagcaggatccagcaactcttccacaaggtggcccagcagcagcggcacctggagaagcagcacctgcgaattcagcatctgcaaagccaggtaaccctaggatcaagggagaaaaggtccctctgatagctggaccccaggttgagagggaggtggtgagaactggacgtgtggctggggacgtggggccaggcaggacctgacaccctcctcccgtcccatcctagtttggcctcctggaccacaagcacctagaccatgaggtggccaagcctgcccgaagaaagaggctgcccgagatggcccagccagttgacccggctcacaatgtcagccgcctgcaccgtgagtgtctgcccctcgatgctctccggtggccacccctaccccgccacttgccattgctggtcctctccttgtcaggtccaccttaaggagaagatgtcctggcctggagtccctgagggctcaccagtctctgggtcaaagctgatgggagcacctccttcctcagccctgacctggcccagccaggccacccaacccttctcctctgcaagccagtgggctgtccctgaagccctgctgatcactgattggaacagaggtggagaaagaggtcttgagggacttgagctgcaggggtgctggagaaggtagaaatggggttcggggtgccatcctgaaggttagaaccttctaggggagggtgtcatggaggagggggtgccaggtagtagtgtttgtagatttgggccctccacaatgtcttggggtatgtgggaattagggctgggaacccccagctcccagaccagaaacactcttgtatcttacaaatccaatgctccaggtccctgacccctcttcctccttcctctcttgttctcctcctccctccccgcccccgccggccccctccccaccgcagaaagtgggcttttgctgccaccacaagttgtaggtgctttattcccaaatctccgttcatctcgaaccacagcatgtccacgtgtgtccgatgcagactcgcggttctctaagttcacggccccacacggtttctctgtggtcctcatccttccctgcatctgtggctgtccacagccaactgggtgaaagtttggatccccctctcacaccctagggtcagtggcaggcttccagcactgtagacctgagggttctctcctccccaagctcccgctccttcccacctccgtgctgcccgcccccaaccccgccaggctagcatctcagcgtggtcagggtcctgtccaccctcccaaagccaccatcccaggatgaggggcttctggagggtgacgggggaaggcacaagtcctggctgggaaatgcggtggaagggggcaggggttctgtgggttcgggactcccagactcttggctcaggcccgccaagtaggagaaagttcagagctgggaaggcgaacagctggcattcatggaagccacactggtggtttggccgcgtgcccatccttactggatgggaggaaagtaggggaaaggggagatgcctgaggggccggaaagcgtcttcctggtcactctgggcccgcccccacccccaccgtgcagactcatttcgacctttcccctacttttccggctgggctgggggcggttcctcccagtctggagcgtctgagcctccagacgtgctcaacgccatcctcccctcctccctccctctttctttcctcaaccctgcctcctctccctctaggagctgggaccccaggcagagcctctgagatgctcctgctcagcacagttcttcccgttctggacctgctgcctcattcattcattcattcattcatttgtcaagactgttttgtttgctttttgagacggagtctcgctctgttgtccaggctggagtgcagtggcgcaatctcagctcactgcaacctctgcctcccactgggttcaagtgattctcctgcctcagcctctcaagtagatgggattataggcgcctaccgccaccacgcccagctaatttttattcatttatcaagtcttttttttttttgagacagagtctcactctgtcacccaggttggagtgcaatggttcaacctcagctgactgcaacctctgcctcccgggttcaagtgattctcctgcctctgcctcctaaggagctgggattactggcgtccaccatcacacccagctaatctttgtatgttttactagagatggggtttggccatgttgaccaggctggtctcaaactcctgaccgatccgcccgcctcccaaagtgctggattacaggcatgaaccactgcgcccggcccattcctcaagtctttattgagcagctgctatgcgctggggcctgcgtggatgctggtgccaggctgtgggcagagctgctccttgtccccagcctcatggagcctccattgagtcagaggagagaccattagatgacctcagtgtccaggagtgggaagtccttgctcaattcccatttggggtgttctggcctggtctgggggtgaggcaaggcagggtgtccttccagaagctgggacccagagagagaagtgaggaaagagagtcccgccgggcgcagtggttcacacctgtaatcccagcactttgggaggccaaggctggcggatcacgaggtcaggagatcgagaccagtctggccaacatggtgaaacccccgtctctacaaataatgcaaaaactagtcgggcgtggtggcacatgcctgtagtcccagctgctccggaggctgcggcagaattgcttgaacctgggaggcggaggttgcagtgagccaagattgcgccactgcactccagcctggcgacagagcgagactccgtatcaaaaaacaaaacaaaaaaagagagtcccaggcagggggaacagcatgtgcaaagtccctgtggcaggacggtgtgtggtacaagggtgggaagaaagcctgtgtgccagagagggtgtgggtacagcatggcaggaggaggatgggcacgggggctggtcctccaggtgccttgtggaccatgctgaggaaggacatagggagccatggaagaggttaggcagatggcagagaggtggtcatgagataagatttgccttttacttccctggtcatccactgttaagcccccagatatgcctggctcctgagaccccccccaggggctgccctcctgtttcaagtctccactttatctcccttcagggctgcccagggattgccaggagctgttccaggttggggagaggcagagtggactatttgaaatccagcctcaggggtctccgccatttttggtgaactgcaagatgacctcaggtagggtgtgttagtccaccaggggcccctctccccataggccctgttgtctttctttaaattgaaaacaaaacaaaacaaaaaaattaaaggcagggtcttgctatgttgcccaagctggtctttttttttttttttttttttgagatggagttttgctcttggagctggagtgcaatgccacgatctcagctcactgaaacctccgcctcccgggttcaagcaattctccagcgtcagcctccccagtagctgagactacaggtgcgagcaaccacacccagctaatttttgtatttttagtagagactgggtttcaccatgttggtctggctggtctcaaactcctgacctcaaatgatttgccgactttggcctcccaaaatgctgggattataggcattagccactgctcctggcctcttttctttaaaatgcagcccttgtctgggtgtagtggctcatatctgtaatcccgacactttgggaggcctatcatgaggatcacttgagcccagaagttcgagaccagcctggacaacatagtgagagctcatctttacaaaaaattttgtaaaagtaaaaaaaaaggccaagtgcagtggctcacacctgtaatcccagcactttgggaggttgaggcagacagatcacgaggtcaggagttcgagaccagcctgaccaacatggtgaaaccccgtctctactaaaaatacaaaaattagccgggcatggtggtgtgcacctgtaatcccagctactcaggtggctgaggcaggagaatctcttgaacctgggaggcagaggttgcagtgagctgagatgacgccaccgcactccagcctgggcgacagagcgagactccgtccccccatcaaaaaaataaaaaagagaaaataaaataaaataaaataaaattcagcccttgctgggcatggtggcacatgcctgtagtcccagctactcgggaggccaaggcaggagggtcacttcagcccaggagttaaaggctgccgtgagccgtgatcacacctgtgaatagccaccgcactccaacctgggcaacacaacaaaacctcgtttctaaaaaaaaacaaaggctgagcacggtggcttacacctgaaatgccagcactttgggaggctgaggcgggcaaatcacttgaggtcagaagttcaagaccagcctggccaacatggtgaaaccctatctctaccaacataaacattagccgggcgtgcctgtgattccagctgcttgtggggctgaagcacaagaatcgcttgagccccgaaggcagaggttgcagtgagctgagattgcggcactgcgatccagcctgggcaacagagtgagactccatctctaaaaaaaaaaaaaaaaaaaaagaaagcttttttttccactgagaagtcctttccattacaagaggttacaacacggggttttacccagttcagctggcccagagaggctttgcagatttccactgcctactccctcccacactcagtccctgctgggttcttgggacaaagatcttcccaaggccagcccataatattcctccctctgacccaccctcctcgagtcctccagggatgagtgagggagctgctgtcttcctgggtttggagggggtttggtgcttggcagccagatgagggagtggggtcgtctgtgaagagggacttcctggtgaccttgtacctttctgggcagatggaggctggacagtaattcagaggcgccacgatggctcagtggacttcaaccggccctgggaagcctacaaggcggggtttggggatccccacggtaggtgtttctagtggggacagaggcaggggaggaagagggaccctcagaagtggccctgcctcatggagtggcctctcccactccaggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggtgagcaggccctgccatgccacacccagccagcagcttccctccttatctttctgctgctctgtcctgccttcaaccccacattgcatctgtttcctgcccccacctcttccttacatgccgtgtgtgtgattgggccactaacttagcctatctggcctcagttttcccatcctgaaaagggtcttgaccgtctttacttttatttacttatgtgtttgtttatttatttatttatgtatttattttttgagacggagtctcactttgtcacccaggctggagtgctttgtggcacgatcttggctcactgcaagctccacctcctgagttcacaccattctcctgcctcagcctcccgagtagctgggactataggtgcccaccaccacgcctggctaatttttttgtatttttagtagagatggggtttcaccgtgttagccaggatggtctcgatctcctgacctcgtgatctgcctgcctcagcctcccaaagtgctgggattacaggcgtgagccaccgcgcccggcctacttatttattttttgagacagagtcccgctgtgtctcccaggctggagtgcaagtgacgtgatcttggctcactgcagcctccgcctcctgggttcaagtgattctcctgcctcagcctcctgagtagctgggattacaggttcccgccaccatgcacagataattgttttgtatttttagtagagacggggtttcaccatgttggccagggtggtcttgaactcctgacctcaagtgatctgcccacctcggcctcccaaagtgttgggattacaggcgtgagccacaatacccggccacaaacatctttataatggtgctccacaggattctttttttttttttttttttgaaacagggtctcactctgttgcctaagctggagtgcagtggtgcgatctcggctcactgcaacctccacctcccgggttcaagcaattctcaaaaaaaaaaaaaaattaggcacggtggctcacacttgcaatcccagcactttgggaggctgaagcgagtggatcacttgagcccaggagaccaatctgagcaacagggcgaaatcctgtctcaattaaaaatacaaaaaactagctgggcatggtggtgcctgcctgtgttcccatctacttgggaggctgagttgggaggatctcttgagcctaggagataaggctgcagtgagctgagactgcgccactgcactcaagcctgggtgacagagtgagacccctgcctcaaaagaaaaagaaaaaatgcaggcatggtggctcacacctgtggtcccagctacttgggaggcccaggtacaagaatcacttgagcccgtaaggttgacgctgcattgagccatcaccacaccactgcactccagcctgggcaatggagccaggccctgtctcaaaaaaaattgtttttaaacttaaaaataaggccgggtgtgggggctcacacctgtaatcccagcactttgggaggccgaggtgggtggatcacctgaggtcaggagttcaaaactagcctggccaacatggtgaaaccctgtctctactaaaaatacaaaaattaggccaggcgcagtgggtcatacctgtaatcccagcactttgggaggctgaggagggtggatcacatgaggtaaggagtttgagaccaacctggccaacatggtgaaaccccctcactactaaaaatacaaaaactagccaggcgtggtggcgggtgcctgtaatcccggctactcaggaggctgaggcatgagaatcccttgaacctgggaggcagaggtgcagtgagccgagattgtgccactgcactccagcctgggagatagagtgagactcagtctcaaaaaaaaagaccaaaaattagccaggtgtggtggcaggcgcctgtaatcccagctactcgggaggctgagggaggagaatcacttaaacctgagagacggaggttgcagtgagctgagatcgcaccactgcactccagcctgggtgacagagtaagactcaatctcaaaaaaaaaaaagtcaagtccaaagcccagcctggtccccaacctgcctcatcctcaaccctatccctatctcctttcagccccatcggtggctcaaagacctgaccatgttccctctcccctgaccccggcaggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcctagcgtcctggctgggcctggtcccaggcccacgaaagacggtgactcttggctctgcccgaggatgtggccgttccctgcctgggcaggggctccaaggaggggccatctggaaacttgtggacagagaagaagaccacgactggagaagccccctttctgagtgcaggggggctgcatgcgttgcctcctgagatcgaggctgcaggatatgctcagactctagaggcgtggaccaaggggcatggagcttcactccttgctggccagggagttggggactcagagggaccacttggggccagccagactggcctcaatggcggactcagtcacattgactgacggggaccagggcttgtgtgggtcgagagcgccctcatggtgctggtgctgttgtgtgtaggtcccctggggacacaagcaggcgccaatggtatctgggcggagctcacagagttcttggaataaaagcaacctcagaacactt

SEQ ID NO: 3 - DNA, 17 bp, synthetic construct
  misc_feature 1..17: note = antisense oligonucleotides hybridizing with human ANGPLT4
  source 1..17: mol_type = other DNA
aggctaagttagtggcc

SEQ ID NO: 4 - DNA, 17 bp, synthetic construct
  misc_feature 1..17: note = antisense oligonucleotides hybridizing with human ANGPLT4
  source 1..17: mol_type = other DNA
gccagataggctaagtt

SEQ ID NO: 5 - DNA, 17 bp, synthetic construct
  misc_feature 1..17: note = antisense oligonucleotides hybridizing with human ANGPLT4
  source 1..17: mol_type = other DNA
ctggaaagaatcggatc

SEQ ID NO: 6 - DNA, 17 bp, synthetic construct
  misc_feature 1..17: note = antisense oligonucleotides hybridizing with human ANGPLT4
  source 1..17: mol_type = other DNA
cgctggaaagaatcgga

SEQ ID NO: 7 - DNA, 17 bp, synthetic construct
  misc_feature 1..17: note = antisense oligonucleotides hybridizing with human ANGPLT4
  source 1..17: mol_type = other DNA
gctagtctcgacagcag

SEQ ID NO: 8 - DNA, 17 bp, synthetic construct
  misc_feature 1..17: note = antisense oligonucleotides hybridizing with human ANGPLT4
  source 1..17: mol_type = other DNA
gatcctcatgataggcc

SEQ ID NO: 9 - DNA, 17 bp, synthetic construct
  misc_feature 1..17: note = antisense oligonucleotides hybridizing with human ANGPLT4
  source 1..17: mol_type = other DNA
ggccgtcggagcaccgc

SEQ ID NO: 10 - DNA, 17 bp, synthetic construct
  misc_feature 1..17: note = antisense oligonucleotides hybridizing with human ANGPLT4
  source 1..17: mol_type = other DNA
catgctgtggttcgaga

SEQ ID NO: 11 - DNA, 17 bp, synthetic construct
  misc_feature 1..17: note = antisense oligonucleotides hybridizing with human ANGPLT4
  source 1..17: mol_type = other DNA
cgtgcgccaggacattc

SEQ ID NO: 12 - DNA, 16 bp, synthetic construct
  misc_feature 1..16: note = antisense oligonucleotides hybridizing with human ANGPLT4
  source 1..16: mol_type = other DNA
cgtgcgccaggacatt

SEQ ID NO: 13 - DNA, 17 bp, synthetic construct
  misc_feature 1..17: note = antisense oligonucleotides hybridizing with human ANGPLT4
  source 1..17: mol_type = other DNA
tccgtgcgccaggacat

SEQ ID NO: 14 - DNA, 17 bp, synthetic construct
  misc_feature 1..17: note = antisense oligonucleotides hybridizing with human ANGPLT4
  source 1..17: mol_type = other DNA
ggagtccgtgcgccagg

SEQ ID NO: 15 - DNA, 17 bp, synthetic construct
  misc_feature 1..17: note = antisense oligonucleotides hybridizing with human ANGPLT4
  source 1..17: mol_type = other DNA
tgcgctccgcgtgttcg

SEQ ID NO: 16 - DNA, 16 bp, synthetic construct
  misc_feature 1..16: note = antisense oligonucleotides hybridizing with human ANGPLT4
  source 1..16: mol_type = other DNA
tgcgctccgcgtgttc

SEQ ID NO: 17 - DNA, 16 bp, synthetic construct
  misc_feature 1..16: note = antisense oligonucleotides hybridizing with human ANGPLT4
  source 1..16: mol_type = other DNA
gtgcgctccgcgtgtt

SEQ ID NO: 18 - DNA, 17 bp, synthetic construct
  misc_feature 1..17: note = antisense oligonucleotides hybridizing with human ANGPLT4
  source 1..17: mol_type = other DNA
gtcgaaatgagtctgca

SEQ ID NO: 19 - DNA, 17 bp, synthetic construct
  misc_feature 1..17: note = antisense oligonucleotides hybridizing with human ANGPLT4
  source 1..17: mol_type = other DNA
gcacgtctggaggctca

SEQ ID NO: 20 - DNA, 17 bp, synthetic construct
  misc_feature 1..17: note = antisense oligonucleotides hybridizing with human ANGPLT4
  source 1..17: mol_type = other DNA
ttgagcacgtctggagg

SEQ ID NO: 21 - DNA, 17 bp, synthetic construct
  misc_feature 1..17: note = antisense oligonucleotides hybridizing with human ANGPLT4
  source 1..17: mol_type = other DNA
ggatcgcagtgccgcaa

SEQ ID NO: 22 - DNA, 14 bp, synthetic construct
  misc_feature 1..14: note = non sense artificical sequence
  source 1..14: mol_type = other DNA
aaaaaaaaaaaaaa

SEQ ID NO: 23 - DNA, 17 bp, synthetic construct
  misc_feature 1..17: note = antisense oligonucleotides hybridizing with human ANGPLT4
  source 1..17: mol_type = other DNA
gctgaattcgcaggtgc

SEQ ID NO: 24 - DNA, 16 bp, synthetic construct
  misc_feature 1..16: note = antisense oligonucleotides hybridizing with human ANGPLT4
  source 1..16: mol_type = other DNA
gatcgcagtgccgcaa

SEQ ID NO: 25 - DNA, 17 bp, synthetic construct
  misc_feature 1..17: note = antisense oligonucleotides hybridizing with human ANGPLT4
  source 1..17: mol_type = other DNA
gtgttgtaacctcttgt

SEQ ID NO: 26 - DNA, 17 bp, synthetic construct
  misc_feature 1..17: note = antisense oligonucleotides hybridizing with human ANGPLT4
  source 1..17: mol_type = other DNA
ccgtgttgtaacctctt

SEQ ID NO: 27 - DNA, 17 bp, synthetic construct
  misc_feature 1..17: note = antisense oligonucleotides hybridizing with human ANGPLT4
  source 1..17: mol_type = other DNA
cctcatggtctaggtgc

SEQ ID NO: 28 - DNA, 17 bp, synthetic construct
  misc_feature 1..17: note = antisense oligonucleotides hybridizing with human ANGPLT4
  source 1..17: mol_type = other DNA
cacctcatggtctaggt

SEQ ID NO: 29 - DNA, 17 bp, synthetic construct
  misc_feature 1..17: note = antisense oligonucleotides hybridizing with human ANGPLT4
  source 1..17: mol_type = other DNA
ggcgcctctgaattact

SEQ ID NO: 30 - DNA, 17 bp, synthetic construct
  misc_feature 1..17: note = antisense oligonucleotides hybridizing with human ANGPLT4
  source 1..17: mol_type = other DNA
cgtggcgcctctgaatt

SEQ ID NO: 31 - DNA, 17 bp, synthetic construct
  misc_feature 1..17: note = antisense oligonucleotides hybridizing with human ANGPLT4
  source 1..17: mol_type = other DNA
tcgtggcgcctctgaat

SEQ ID NO: 32 - DNA, 17 bp, synthetic construct
  misc_feature 1..17: note = antisense oligonucleotides hybridizing with human ANGPLT4
  source 1..17: mol_type = other DNA
ccgccttgtaggcttcc

SEQ ID NO: 33 - DNA, 17 bp, synthetic construct
  misc_feature 1..17: note = antisense oligonucleotides hybridizing with human ANGPLT4
  source 1..17: mol_type = other DNA
actcggcgttgccatcc

SEQ ID NO: 34 - DNA, 17 bp, synthetic construct
  misc_feature 1..17: note = antisense oligonucleotides hybridizing with human ANGPLT4
  source 1..17: mol_type = other DNA
gccgtgtcctcgccacc

SEQ ID NO: 35 - DNA, 17 bp, synthetic construct
  misc_feature 1..17: note = antisense oligonucleotides hybridizing with human ANGPLT4
  source 1..17: mol_type = other DNA
gaggtcgtgatcctggt

SEQ ID NO: 36 - DNA, 17 bp, synthetic construct
  misc_feature 1..17: note = antisense oligonucleotides hybridizing with human ANGPLT4
  source 1..17: mol_type = other DNA
gcggaggtcgtgatcct

SEQ ID NO: 37 - DNA, 17 bp, synthetic construct
  misc_feature 1..17: note = antisense oligonucleotides hybridizing with human ANGPLT4
  source 1..17: mol_type = other DNA
cctgcggaggtcgtgat

SEQ ID NO: 38 - DNA, 17 bp, synthetic construct
  misc_feature 1..17: note = antisense oligonucleotides hybridizing with human ANGPLT4
  source 1..17: mol_type = other DNA
ctcttggcgcagttctt

SEQ ID NO: 39 - DNA, 17 bp, synthetic construct
  misc_feature 1..17: note = antisense oligonucleotides hybridizing with human ANGPLT4
  source 1..17: mol_type = other DNA
gctcttggcgcagttct

SEQ ID NO: 40 - DNA, 17 bp, synthetic construct
  misc_feature 1..17: note = antisense oligonucleotides hybridizing with human ANGPLT4
  source 1..17: mol_type = other DNA
ggctcttggcgcagttc

SEQ ID NO: 41 - DNA, 17 bp, synthetic construct
  misc_feature 1..17: note = antisense oligonucleotides hybridizing with human ANGPLT4
  source 1..17: mol_type = other DNA
ggtgccaaaccaccagc

SEQ ID NO: 42 - DNA, 17 bp, synthetic construct
  misc_feature 1..17: note = antisense oligonucleotides hybridizing with human ANGPLT4
  source 1..17: mol_type = other DNA
ggagcggaagtactggc

SEQ ID NO: 43 - DNA, 17 bp, synthetic construct
  misc_feature 1..17: note = antisense oligonucleotides hybridizing with human ANGPLT4
  source 1..17: mol_type = other DNA
atggagcggaagtactg

SEQ ID NO: 44 - DNA, 17 bp, synthetic construct
  misc_feature 1..17: note = antisense oligonucleotides hybridizing with human ANGPLT4
  source 1..17: mol_type = other DNA
ggatggagcggaagtac

SEQ ID NO: 45 - DNA, 17 bp, synthetic construct
  misc_feature 1..17: note = antisense oligonucleotides hybridizing with human ANGPLT4
  source 1..17: mol_type = other DNA
ggctggatcaacatggt

SEQ ID NO: 46 - DNA, 17 bp, synthetic construct
  misc_feature 1..17: note = antisense oligonucleotides hybridizing with human ANGPLT4
  source 1..17: mol_type = other DNA
atgtgactgagtccgcc

SEQ ID NO: 47 - DNA, 17 bp, synthetic construct
  misc_feature 1..17: note = antisense oligonucleotides hybridizing with human ANGPLT4
  source 1..17: mol_type = other DNA
caatgtgactgagtccg

SEQ ID NO: 48 - DNA, 17 bp, synthetic construct
  misc_feature 1..17: note = antisense oligonucleotides hybridizing with human ANGPLT4
  source 1..17: mol_type = other DNA
gaactctgtgagctccg

SEQ ID NO: 49 - DNA, 17 bp, synthetic construct
  misc_feature 1..17: note = antisense oligonucleotides hybridizing with human ANGPLT4
  source 1..17: mol_type = other DNA
ctggtggactaacacac

SEQ ID NO: 50 - DNA, 17 bp, synthetic construct
  misc_feature 1..17: note = antisense oligonucleotides hybridizing with human ANGPLT4
  source 1..17: mol_type = other DNA
tggacctgacaaggaga

SEQ ID NO: 51 - DNA, 16 bp, synthetic construct
  misc_feature 1..16: note = antisense oligonucleotides hybridizing with human ANGPLT4
  source 1..16: mol_type = other DNA
cacacggcatgtaagg

SEQ ID NO: 52 - DNA, 17 bp, synthetic construct
  misc_feature 1..17: note = antisense oligonucleotides hybridizing with human ANGPLT4
  source 1..17: mol_type = other DNA
aggctagtctcgacagc

SEQ ID NO: 53 - DNA, 17 bp, synthetic construct
  misc_feature 1..17: note = antisense oligonucleotides hybridizing with human ANGPLT4
  source 1..17: mol_type = other DNA
gatcgcagtgccgcaat

SEQ ID NO: 54 - DNA, 17 bp, synthetic construct
  misc_feature 1..17: note = antisense oligonucleotides hybridizing with human ANGPLT4
  source 1..17: mol_type = other DNA
cgtgttgtaacctcttg

SEQ ID NO: 55 - DNA, 15 bp, synthetic construct
  misc_feature 1..15: note = antisense oligonucleotides hybridizing with human ANGPLT4
  source 1..15: mol_type = other DNA
gatcgcagtgccgca

SEQ ID NO: 56 - DNA, 15 bp, synthetic construct
  misc_feature 1..15: note = antisense oligonucleotides hybridizing with human ANGPLT4
  source 1..15: mol_type = other DNA
ggatcgcagtgccgc

SEQ ID NO: 57 - DNA, 18 bp, synthetic construct
  misc_feature 1..18: note = oligonucleotide negative control
  modified_base 1..17: mod_base = OTHER; note = nucleotide may be modified with phosphorothioate (PTO)
  modified_base 1..3: mod_base = OTHER; note = nucleotide may be modified as locked nucleic acid (LNA)
  modified_base 15..17: mod_base = OTHER; note = nucleotide may be modified as locked nucleic acid (LNA)
  source 1..18: mol_type = other DNA
cgtttaggctatgtactt

SEQ ID NO: 58 - DNA, 1916 bp, Mus musculus
  source 1..1916: mol_type = other DNA
gctttataaagtggggctttaggtgcaaccgtgaaacgcttatgagctacgggctccagatcttcttctgcaccagagcaagtctaagtctgagccggctcccccagaactccagctgctgggtcttgaactcctgcgttccggagtcctagcgttgctgcacccaaggccacccccagaatcatgcgctgcgctccgacagcaggcgctgccctggtgctatgcgcggctactgcggggcttttgagcgcgcaagggcgccctgcacagccagagccaccgcgctttgcatcctgggacgagatgaacttgctggctcacgggctgctacagctcggccatgggctgcgcgaacacgtggagcgcacccgtgggcagctgggcgcgctggagcgccgcatggctgcctgtggtaacgcttgtcaggggcccaagggaaaagatgcacccttcaaagactccgaggatagagtccctgaaggccagactcctgagactctgcagagtttgcagactcagctcaaggctcaaaacagcaagatccagcaattgttccagaaggtggcccagcagcagagatacctatcaaagcagaatctgagaatacagaatcttcagagccagatagacctcttggcccccacgcacctagacaatggagtagacaagacttcgaggggaaagaggcttcccaagatgacccagctcattggcttgactcccaacgccacccacttacacaggccgccccgggactgccaggaactcttccaagaaggggagcggcacagtggacttttccagatccagcctctggggtctccaccatttttggtcaactgtgagatgacttcagatggaggctggacagtgattcagagacgcctgaacggctctgtggacttcaaccagtcctgggaagcctacaaggatggcttcggagatccccaaggcgagttctggctgggcctggaaaagatgcacagcatcacagggaaccgaggaagccaattggctgtgcagctccaggactgggatggcaatgccaaattgctccaatttcccatccatttggggggtgaggacacagcctacagcctgcagctcactgagcccacggccaatgagctgggtgccaccaatgtttcccccaatggcctttccctgcccttctctacttgggaccaagaccatgacctccgtggggaccttaactgtgccaagagcctctctggtggctggtggtttggtacctgtagccattccaatctcaatggacaatacttccactctatcccacggcaacggcaggagcgtaaaaagggtatcttctggaaaacatggaagggccgctactatcctctgcaggctaccaccctgctgatccagcccatggaggctacagcagcctcttagcctcctcactggagcctggttccaggcctaagaagacagtgactttggttgtggccctgagatttggccattctctgctgggggcaggagctctaagtagggctatctgcgtcttgtggacaaagaagaagcccgtaactggagagactggaggaccccttttccgtgttggggtctgcaagcattgttgtctgaaacagtcagagcaacaggaaacaaatggcccagatccagaaaacatgggctcgaggggcactgaatatcacttctcgcctaccagagaagttggggatgcagagggaccactacagtccaactagctgggcccttaatggcggactcagtcatattgactgactggagacagggtgccaggagccctggatacactcatggtgctgttgtaggtgctgtggatgcacaggtgctaactgtggttcccaggcacagctcacagcattcttacaataaaaacaacctcagaacaaaaaaaaaaaaaa

SEQ ID NO: 59 - DNA, 7826 bp, Mus musculus
  source 1..7826: mol_type = other DNA
gctttataaagtggggctttaggtgcaaccgtgaaacgcttatgagctacgggctccagatcttcttctgcaccagagcaagtctaagtctgagccggctcccccagaactccagctgctgggtcttgaactcctgcgttccggagtcctagcgttgctgcacccaaggccacccccagaatcatgcgctgcgctccgacagcaggcgctgccctggtgctatgcgcggctactgcggggcttttgagcgcgcaagggcgccctgcacagccagagccaccgcgctttgcatcctgggacgagatgaacttgctggctcacgggctgctacagctcggccatgggctgcgcgaacacgtggagcgcacccgtgggcagctgggcgcgctggagcgccgcatggctgcctgtggtaacgcttgtcaggggcccaagggaaaagatgcacccttcaaagactccgaggatagagtccctgaaggccagactcctgagactctgcagagtttgcaggtaggcgcatctactaggactctgactctacttagactaacgagtctagtactccaggacctgggtggagaagggcctcagagcagactgtgaaagggtagaagggagggtgaataagaggaggttgccgacataaagccaggtcttcactagccttgcatatcttcagactcagctcaaggctcaaaacagcaagatccagcaattgttccagaaggtggcccagcagcagagatacctatcaaagcagaatctgagaatacagaatcttcagagccaggtaattaccccagcaattggcccaaatgggaaagaggaaacaaagtgggggtgaaatgtagtgatgaaatgtggctaagcagttgttatggcatgagtcaaaagatctaaccacccgtttcctgcccggtcttagatagacctcttggcccccacgcacctagacaatggagtagacaagacttcgaggggaaagaggcttcccaagatgacccagctcattggcttgactcccaacgccacccacttacacagtgagtatctctggtctttgctgtcatctggcaactccccaaccaccccacttgccattccaatccttagccttcttgtcaggtccaccttaataggaaagaaagagattctggccttgggttcctgtgggctcactaggctctcatcaagggtatttttatatattgcaccctcttcacttatgatcctgcttggccagaccaccccaccattctttcccagcaagaatcttcccaaaatcttattagccgttaaccagaacaagtagaggaaggcagatcataaggagctaaagtttcaggggttctgggagatggtggtgtagctcagggacatcatgcaaaggaatccagggctgtgtgtggatgtgtgtgcgttcccgaggtttggaaccctccaacatctcttgatgtaactataaaagggaaaaccagagtaagcacacccccaccccggctcctagactagacacatcctcttacatcttacagatacagatcctttgtcctgtatgcttccatccagtcacatacacatatatacataacagtttcgatcacaatttctaggtcttagattgtggatgtccatgcccaatactttgtttcaaattgaagtgtgtgtggagctacgcagtgcgggatgggcgggtgggggatggggggcacagagtcagaggctcccagttccaaccctgagttcatgcctattcgttcacccttcttgacatctgtggctctccacagccaactaggtgaaagtttggatatccccttcacaccctaaggctgcttgctggctttcagcccaatcctccttagacttaattcctcccctggcttttacttaggcaagtacctaggcatggcgagggtcatggatgagcttttccagggtcggcaggcataggttctggatgggtgatgtggtggaagagaagagctgagggggtggttttgtggctttggaactcccagactcagcctagccaagtagaggaaagttcagagctggagagacaaacagctggccctaatgggaaccacatgggtggtttggcagtgtgcctgtcccgattggatgagaggaaagtaggggaaagggagctagctgcccgagggattggaaagtatacttcccatctttctgggtctgcccccactcctggcagtgcagacggagctggctggcagaaagtgcctgcatgcccctcccccagtgacctttctcctactttccctggctgggctgggggtggttcttcctgaatttagaggacttgtcaaggccaagttctttctctcctcctctcctctttttcttttcttcctcttcccccttcttctggtaacagacatagccgtatcccacacacacacccagcctcagtctcccagtcccactgtcagcatcttctactaattcagagtacccctttgctcagtcagctcagctcttgccttaccccagatccaccccgtccattgtttgaagtcttcactgaaacactgactgtggggtaggctttaggtggttacaggtccctactgtggacaaacttcctcctcgtctctggcctcagtgggaggagtggggacatgcctccagatgactcttcatcagcgacaatacctctgttcacttttgggagtctaacagggcttgagagagtggaatagcatccttttagaagctgagacccagagaaaggaatgggaaaatggaatccctggcatcaggaccatgtgctgattgtcaataacaggaaggtgtgatacatgggaaaaaggctcagggagagaagcctttgtgacctgagagggtagacaagcagagtgggaagagtgaagattggtcaagggactgaccgttccttagtgtgccttgaggaccctactggagaatgctataatgaggcaggaacgagtttaggcaaatcaccctggggctcatcctctaccacccagccttcacactcaccagtctcctgaaatcactgggggcttctctcctgcttcactctctccctttgtctactttcagggccgccccgggactgccaggaactcttccaagaaggggagcggcacagtggacttttccagatccagcctctggggtctccaccatttttggtcaactgtgagatgacttcaggtaggatgtgctgacacccacccccccaaggagcctccttgccatgcacagccctgatgtcataaaataaagccatgtatagatcaataattacaagcacttacctagcttgtataagctcctgagatccttccacagtaacacacaccctattatgcagcttgtaggatgtagcctgtaggaggtatatagataatcttcttccctattgacaggcatcgcaagctcagtgataggtttctgtggttaaggtgtacagctgtccaggcctagtcatctacaaggtgagatcaaggccagcctgggtaacttagtataaccctgtcatgcaataacaaaatataagtgagctgggggcatagcttaatgggaaagtgcctcctacaatgtacaagaccctaaattcaatgcatagaaccacagttggatatggtgacttgtgcctatatttataatcccaatacttgggaggttgaggcaaaatgattgttgaaagtgcaaggccagccttatctcagtcttcatgggctgggaataaagctcagttggtaaagcgcttccattgaatgtatgaagccctggcttccatctccaacactgcataaaatgggtatgatagtgcaagtctgctacccaaaactctggaggtggtagcaggctgatgaggagtttgagatcatccacagttacatagtgagttgaaggacagcttgggctataggaaaaaggtagtctaaggtagagactctgtttcaaacaacaaaacacatatactcctggttcctgcaggctcctaggatagaagtctgttctgatcccaccccctgcggggactgatgagtgtgttgcccttcctgggattgtatgggagcatgatattaggcttgcaagctaggtagggctatcagtttccctgtgagaagggacttctggtgactttttgtctttctgggcagatggaggctggacagtgattcagagacgcctgaacggctctgtggacttcaaccagtcctgggaagcctacaaggatggcttcggagatccccaaggtaggtgtttctctggaggccatagtcatggaaggagcaaagggtgcgagagttgagcctggctcacatatgatttctcttgattcaggcgagttctggctgggcctggaaaagatgcacagcatcacagggaaccgaggaagccaattggctgtgcagctccaggactgggatggcaatgccaaattgctccaatttcccatccatttggggggtgaggacacagcctacagcctgcagctcactgagcccacggccaatgagctgggtgccaccaatgtttcccccaatggcctttccctgcccttctctacttgggaccaagaccatgacctccgtggggaccttaactgtgccaagagcctctctggtaagcaggttttattcccactgtagcctacattcagtcccaaagtgtgtatccatgccctgccccccttttctccttgcctcctgtgtgcctatgggccagtcattgaattaatctctgctgttttcagtttccctgtctttaagtgaacataatcatggtgcctgcttttgaaaattcaactgagaacatttgtgggtgtgtagctgccacagggcaaagccaagacaaacccttgtcttattcttttcaattctgaggtatagggtcttgtgccattctggcacagagctacaccctgacccctggtgttttgtttttgttgtctgtttgactcagggtcttatatagctctagctggccttgaacttgttatacagctaaagataagcttaaagggtcagcctcggagctggaaagatggctcagtggttaagagcactgactgctcttcccaggggtcctgagttcaattcccagcaactccatggtggctcacaaccacctccaatggggtctgatgccctcttctggtgtgtctgaagagagcaatggtgtattcatatacattaaataaataaaccatttaaaaagggaggggggcatcagcctctaactcctgagtgttggggtaacaggcatgtgccattgtgctcctgaggatcaatcccacaattccatgtctggtaggcaagcactgtaacaaatgagctacagtactagctctttttctgcttcacttattcagtcaggatcaaattataaagagcaggctggcctcaaacttcctatcatactgctgagcactgtgactacaaatttctacccacaaactgcttcatagtgtagctcatttggcagagaacctacctaacatgagtgaaggcctaggtttcattacccataccatataaaacacacatggttgtgcaatgtcaaccatagactcagaaagaggaggcaggtaaagcagacattttaaattgtctttggccacgtggagagtttgaggccagcccgataggagaccccgtcacaaaacaaaaataggacttatgagatggctcagccagtaacagtgtttgcctacaatcctgaagacctaagtttgatctccaagactcacatagtagaaagagatcactggctcgctcccacaagctgtcctctgaccttcatacccactgtggcatgtacacccacacacatatacacgggaacacaaagtaaataaatgtaataaattctaaaggtgtaaaatccaaagttcatcttgtctccaactgtgctctctctctctctctctcctctctctctctctctctctctcagtatggaaacccaagaccttgaccacatctcctgttctttaaccctataggtggctggtggtttggtacctgtagccattccaatctcaatggacaatacttccactctatcccacggcaacggcaggagcgtaaaaagggtatcttctggaaaacatggaagggccgctactatcctctgcaggctaccaccctgctgatccagcccatggaggctacagcagcctcttagcctcctcactggagcctggttccaggcctaagaagacagtgactttggttgtggccctgagatttggccattctctgctgggggcaggagctctaagtagggctatctgcgtcttgtggacaaagaagaagcccgtaactggagagactggaggaccccttttccgtgttggggtctgcaagcattgttgtctgaaacagtcagagcaacaggaaacaaatggcccagatccagaaaacatgggctcgaggggcactgaatatcacttctcgcctaccagagaagttggggatgcagagggaccactacagtccaactagctgggcccttaatggcggactcagtcatattgactgactggagacagggtgccaggagccctggatacactcatggtgctgttgtaggtgctgtggatgcacaggtgctaactgtggttcccaggcacagctcacagcattcttacaataaaaacaacctcagaacattttgttctctgttgtttcatttgatttttcaaagtgaatagtttcaaagttcaagtaaacatgttcctcggacatcgtattcccagatccgttaaggacagaactggttggctattccttcccagtgatttcagcttccttcttttagtatttcatgatgtgagcctctcttgtttatatttcaaaatctcaaaacaggtccagggagatggctcagtgattaggaacattcgttcttccagagggcctgggtttggttcccagcctttacatagcagctaacaatagcctataactctacttcaagggggtccaatgccgcctctgacctctactggctcctgtacaaatgtgataatatgcaaatatttaggcactcatatagaaaagaaagtattgtttaaaaaacagagttagggaaatggcttaatgattccaaacacttgctgtctaagcctggtgactgagttcaagcacccaaaaggtcaaaggacagaacagattccgcaaagttggcctttgaccaccacatacaagccatggcacttgggctgtcacacagatatatacacaataattgttttttttttaatttttaaaagttaaaaatcatataactcatgcagtccaatcattagggaagttggtgatagagacaagtaaaaagtctttctgatcagtactcggtggggtacacacagatctccagcccctgggagctggacataggagctttgcaaactctaagccagactaggcaatacaacaagatcctgtatcacaaacagggtgagtactgtagatgtagctcaactggacatacgaagcatgcacagaccctgggttctttcccagcagcactgcataaataaagtatagcgctagataacactgtaatgctcgtatttgggaggtggaggcatgaggattagaagttgaggtaacctgggctacaagaaagaaaaagaagagagaaaagcaaaatgagatgaagttaattttagaaatctaaacccgatgtcatattccatacttgtaatttcagcacttggggagctgaggcagggggaccagaattaccatgagtttgagtccaacttaggctatatagtaagaccttgactaataaaaacaaagacgttaaaaaag

SEQ ID NO: 60 - DNA, 15 bp, synthetic construct
  misc_feature 1..15: note = antisense oligonucleotides hybridizing with murine ANGPLT4
  source 1..15: mol_type = other DNA
gttgcacctaaagcc

SEQ ID NO: 61 - DNA, 17 bp, synthetic construct
  misc_feature 1..17: note = antisense oligonucleotides hybridizing with murine ANGPLT4
  source 1..17: mol_type = other DNA
acggttgcacctaaagc

SEQ ID NO: 62 - DNA, 16 bp, synthetic construct
  misc_feature 1..16: note = antisense oligonucleotides hybridizing with murine ANGPLT4
  source 1..16: mol_type = other DNA
acggttgcacctaaag

SEQ ID NO: 63 - DNA, 16 bp, synthetic construct
  misc_feature 1..16: note = antisense oligonucleotides hybridizing with murine ANGPLT4
  source 1..16: mol_type = other DNA
tcacggttgcacctaa

SEQ ID NO: 64 - DNA, 16 bp, synthetic construct
  misc_feature 1..16: note = antisense oligonucleotides hybridizing with murine ANGPLT4
  source 1..16: mol_type = other DNA
ttcacggttgcaccta

SEQ ID NO: 65 - DNA, 17 bp, synthetic construct
  misc_feature 1..17: note = antisense oligonucleotides hybridizing with murine ANGPLT4
  source 1..17: mol_type = other DNA
ataagcgtttcacggtt

SEQ ID NO: 66 - DNA, 17 bp, synthetic construct
  misc_feature 1..17: note = antisense oligonucleotides hybridizing with murine ANGPLT4
  source 1..17: mol_type = other DNA
cataagcgtttcacggt

SEQ ID NO: 67 - DNA, 17 bp, synthetic construct
  misc_feature 1..17: note = antisense oligonucleotides hybridizing with murine ANGPLT4
  source 1..17: mol_type = other DNA
cgtagctcataagcgtt

SEQ ID NO: 68 - DNA, 16 bp, synthetic construct
  misc_feature 1..16: note = antisense oligonucleotides hybridizing with murine ANGPLT4
  source 1..16: mol_type = other DNA
gctaggactccggaac

SEQ ID NO: 69 - DNA, 17 bp, synthetic construct
  misc_feature 1..17: note = antisense oligonucleotides hybridizing with murine ANGPLT4
  source 1..17: mol_type = other DNA
acgctaggactccggaa

SEQ ID NO: 70 - DNA, 15 bp, synthetic construct
  misc_feature 1..15: note = antisense oligonucleotides hybridizing with murine ANGPLT4
  source 1..15: mol_type = other DNA
aacgctaggactccg

SEQ ID NO: 71 - DNA, 15 bp, synthetic construct
  misc_feature 1..15: note = antisense oligonucleotides hybridizing with murine ANGPLT4
  source 1..15: mol_type = other DNA
agcgcatgattctgg

SEQ ID NO: 72 - DNA, 17 bp, synthetic construct
  misc_feature 1..17: note = antisense oligonucleotides hybridizing with murine ANGPLT4
  source 1..17: mol_type = other DNA
cggagcgcagcgcatga

SEQ ID NO: 73 - DNA, 17 bp, synthetic construct
  misc_feature 1..17: note = antisense oligonucleotides hybridizing with murine ANGPLT4
  source 1..17: mol_type = other DNA
agccgcgcatagcacca

SEQ ID NO: 74 - DNA, 16 bp, synthetic construct
  misc_feature 1..16: note = antisense oligonucleotides hybridizing with murine ANGPLT4
  source 1..16: mol_type = other DNA
tagccgcgcatagcac

SEQ ID NO: 75 - DNA, 17 bp, synthetic construct
  misc_feature 1..17: note = antisense oligonucleotides hybridizing with murine ANGPLT4
  source 1..17: mol_type = other DNA
agtagccgcgcatagca

SEQ ID NO: 76 - DNA, 17 bp, synthetic construct
  misc_feature 1..17: note = antisense oligonucleotides hybridizing with murine ANGPLT4
  source 1..17: mol_type = other DNA
cagtagccgcgcatagc

SEQ ID NO: 77 - DNA, 16 bp, synthetic construct
  misc_feature 1..16: note = antisense oligonucleotides hybridizing with murine ANGPLT4
  source 1..16: mol_type = other DNA
cagtagccgcgcatag

SEQ ID NO: 78 - DNA, 16 bp, synthetic construct
  misc_feature 1..16: note = antisense oligonucleotides hybridizing with murine ANGPLT4
  source 1..16: mol_type = other DNA
gcagtagccgcgcata

SEQ ID NO: 79 - DNA, 15 bp, synthetic construct
  misc_feature 1..15: note = antisense oligonucleotides hybridizing with murine ANGPLT4
  source 1..15: mol_type = other DNA
ccttgcgcgctcaaa

SEQ ID NO: 80 - DNA, 16 bp, synthetic construct
  misc_feature 1..16: note = antisense oligonucleotides hybridizing with murine ANGPLT4
  source 1..16: mol_type = other DNA
gatgcaaagcgcggtg

SEQ ID NO: 81 - DNA, 17 bp, synthetic construct
  misc_feature 1..17: note = antisense oligonucleotides hybridizing with murine ANGPLT4
  source 1..17: mol_type = other DNA
aggatgcaaagcgcggt

SEQ ID NO: 82 - DNA, 16 bp, synthetic construct
  misc_feature 1..16: note = antisense oligonucleotides hybridizing with murine ANGPLT4
  source 1..16: mol_type = other DNA
ccacgtgttcgcgcag

SEQ ID NO: 83 - DNA, 17 bp, synthetic construct
  misc_feature 1..17: note = antisense oligonucleotides hybridizing with murine ANGPLT4
  source 1..17: mol_type = other DNA
ctccacgtgttcgcgca

SEQ ID NO: 84 - DNA, 17 bp, synthetic construct
  misc_feature 1..17: note = antisense oligonucleotides hybridizing with murine ANGPLT4
  source 1..17: mol_type = other DNA
tgcgctccacgtgttcg

SEQ ID NO: 85 - DNA, 16 bp, synthetic construct
  misc_feature 1..16: note = antisense oligonucleotides hybridizing with murine ANGPLT4
  source 1..16: mol_type = other DNA
tgcgctccacgtgttc

SEQ ID NO: 86 - DNA, 15 bp, synthetic construct
  misc_feature 1..15: note = antisense oligonucleotides hybridizing with murine ANGPLT4
  source 1..15: mol_type = other DNA
tgcgctccacgtgtt

SEQ ID NO: 87 - DNA, 16 bp, synthetic construct
  misc_feature 1..16: note = antisense oligonucleotides hybridizing with murine ANGPLT4
  source 1..16: mol_type = other DNA
atgcggcgctccagcg

SEQ ID NO: 88 - DNA, 17 bp, synthetic construct
  misc_feature 1..17: note = antisense oligonucleotides hybridizing with murine ANGPLT4
  source 1..17: mol_type = other DNA
ctgacaagcgttaccac

SEQ ID NO: 89 - DNA, 17 bp, synthetic construct
  misc_feature 1..17: note = antisense oligonucleotides hybridizing with murine ANGPLT4
  source 1..17: mol_type = other DNA
cctgacaagcgttacca

SEQ ID NO: 90 - DNA, 15 bp, synthetic construct
  misc_feature 1..15: note = antisense oligonucleotides hybridizing with murine ANGPLT4
  source 1..15: mol_type = other DNA
cctgacaagcgttac

SEQ ID NO: 91 - DNA, 16 bp, synthetic construct
  misc_feature 1..16: note = antisense oligonucleotides hybridizing with murine ANGPLT4
  source 1..16: mol_type = other DNA
attgtctaggtgcgtg

SEQ ID NO: 92 - DNA, 17 bp, synthetic construct
  misc_feature 1..17: note = antisense oligonucleotides hybridizing with murine ANGPLT4
  source 1..17: mol_type = other DNA
ccattgtctaggtgcgt

SEQ ID NO: 93 - DNA, 17 bp, synthetic construct
  misc_feature 1..17: note = antisense oligonucleotides hybridizing with murine ANGPLT4
  source 1..17: mol_type = other DNA
cgttcaggcgtctctga

SEQ ID NO: 94 - DNA, 16 bp, synthetic construct
  misc_feature 1..16: note = antisense oligonucleotides hybridizing with murine ANGPLT4
  source 1..16: mol_type = other DNA
agagccgttcaggcgt

SEQ ID NO: 95 - DNA, 15 bp, synthetic construct
  misc_feature 1..15: note = antisense oligonucleotides hybridizing with murine ANGPLT4
  source 1..15: mol_type = other DNA
gctcattggccgtgg

SEQ ID NO: 96 - DNA, 17 bp, synthetic construct
  misc_feature 1..17: note = antisense oligonucleotides hybridizing with murine ANGPLT4
  source 1..17: mol_type = other DNA
agtggaagtattgtcca

SEQ ID NO: 97 - DNA, 17 bp, synthetic construct
  misc_feature 1..17: note = antisense oligonucleotides hybridizing with murine ANGPLT4
  source 1..17: mol_type = other DNA
tacgctcctgccgttgc

SEQ ID NO: 98 - DNA, 17 bp, synthetic construct
  misc_feature 1..17: note = antisense oligonucleotides hybridizing with murine ANGPLT4
  source 1..17: mol_type = other DNA
ttacgctcctgccgttg

SEQ ID NO: 99 - DNA, 17 bp, synthetic construct
  misc_feature 1..17: note = antisense oligonucleotides hybridizing with murine ANGPLT4
  source 1..17: mol_type = other DNA
tttttacgctcctgccg

SEQ ID NO: 100 - DNA, 15 bp, synthetic construct
  misc_feature 1..15: note = antisense oligonucleotides hybridizing with murine ANGPLT4
  source 1..15: mol_type = other DNA
ttttacgctcctgcc

SEQ ID NO: 101 - DNA, 17 bp, synthetic construct
  misc_feature 1..17: note = antisense oligonucleotides hybridizing with murine ANGPLT4
  source 1..17: mol_type = other DNA
agaggatagtagcggcc

SEQ ID NO: 102 - DNA, 17 bp, synthetic construct
  misc_feature 1..17: note = antisense oligonucleotides hybridizing with murine ANGPLT4
  source 1..17: mol_type = other DNA
acaagacgcagatagcc

SEQ ID NO: 103 - DNA, 17 bp, synthetic construct
  misc_feature 1..17: note = antisense oligonucleotides hybridizing with murine ANGPLT4
  source 1..17: mol_type = other DNA
ggcgagaagtgatattc

SEQ ID NO: 104 - DNA, 17 bp, synthetic construct
  misc_feature 1..17: note = antisense oligonucleotides hybridizing with murine ANGPLT4
  source 1..17: mol_type = other DNA
taggcgagaagtgatat

SEQ ID NO: 105 - DNA, 16 bp, synthetic construct
  misc_feature 1..16: note = antisense oligonucleotides hybridizing with murine ANGPLT4
  source 1..16: mol_type = other DNA
gtaggcgagaagtgat

SEQ ID NO: 106 - DNA, 16 bp, synthetic construct
  misc_feature 1..16: note = antisense oligonucleotides hybridizing with murine ANGPLT4
  source 1..16: mol_type = other DNA
gagtccgccattaagg

SEQ ID NO: 107 - DNA, 17 bp, synthetic construct
  misc_feature 1..17: note = antisense oligonucleotides hybridizing with murine ANGPLT4
  source 1..17: mol_type = other DNA
actgagtccgccattaa

SEQ ID NO: 108 - DNA, 17 bp, synthetic construct
  misc_feature 1..17: note = antisense oligonucleotides hybridizing with murine ANGPLT4
  source 1..17: mol_type = other DNA
atatgactgagtccgcc

SEQ ID NO: 109 - DNA, 17 bp, synthetic construct
  misc_feature 1..17: note = antisense oligonucleotides hybridizing with murine ANGPLT4
  source 1..17: mol_type = other DNA
aatatgactgagtccgc

SEQ ID NO: 110 - DNA, 16 bp, synthetic construct
  misc_feature 1..16: note = antisense oligonucleotides hybridizing with murine ANGPLT4
  source 1..16: mol_type = other DNA
cgctaggactccggaa

SEQ ID NO: 111 - DNA, 17 bp, synthetic construct
  misc_feature 1..17: note = antisense oligonucleotides hybridizing with murine ANGPLT4
  source 1..17: mol_type = other DNA
aacgctaggactccgga

SEQ ID NO: 112 - DNA, 16 bp, synthetic construct
  misc_feature 1..16: note = antisense oligonucleotides hybridizing with murine ANGPLT4
  source 1..16: mol_type = other DNA
aacgctaggactccgg

SEQ ID NO: 113 - DNA, 16 bp, synthetic construct
  misc_feature 1..16: note = antisense oligonucleotides hybridizing with murine ANGPLT4
  source 1..16: mol_type = other DNA
caacgctaggactccg

SEQ ID NO: 114 - DNA, 17 bp, synthetic construct
  misc_feature 1..17: note = antisense oligonucleotides hybridizing with murine ANGPLT4
  source 1..17: mol_type = other DNA
gcaacgctaggactccg

SEQ ID NO: 115 - DNA, 15 bp, synthetic construct
  misc_feature 1..15: note = antisense oligonucleotides hybridizing with murine ANGPLT4
  source 1..15: mol_type = other DNA
gtagccgcgcatagc

SEQ ID NO: 116 - DNA, 16 bp, synthetic construct
  misc_feature 1..16: note = antisense oligonucleotides hybridizing with murine ANGPLT4
  source 1..16: mol_type = other DNA
agtagccgcgcatagc

SEQ ID NO: 117 - DNA, 15 bp, synthetic construct
  misc_feature 1..15: note = antisense oligonucleotides hybridizing with murine ANGPLT4
  source 1..15: mol_type = other DNA
agtagccgcgcatag

SEQ ID NO: 118 - DNA, 17 bp, synthetic construct
  misc_feature 1..17: note = antisense oligonucleotides hybridizing with murine ANGPLT4
  source 1..17: mol_type = other DNA
gcagtagccgcgcatag

SEQ ID NO: 119 - DNA, 15 bp, synthetic construct
  misc_feature 1..15: note = antisense oligonucleotides hybridizing with murine ANGPLT4
  source 1..15: mol_type = other DNA
cagtagccgcgcata

SEQ ID NO: 120 - DNA, 17 bp, synthetic construct
  misc_feature 1..17: note = antisense oligonucleotides hybridizing with murine ANGPLT4
  source 1..17: mol_type = other DNA
ccgcagtagccgcgcat

SEQ ID NO: 121 - DNA, 16 bp, synthetic construct
  misc_feature 1..16: note = antisense oligonucleotides hybridizing with murine ANGPLT4
  source 1..16: mol_type = other DNA
ccttgcgcgctcaaaa

SEQ ID NO: 122 - DNA, 16 bp, synthetic construct
  misc_feature 1..16: note = antisense oligonucleotides hybridizing with murine ANGPLT4
  source 1..16: mol_type = other DNA
ccttgcgcgctcaaaa

SEQ ID NO: 123 - DNA, 16 bp, synthetic construct
  misc_feature 1..16: note = antisense oligonucleotides hybridizing with murine ANGPLT4
  source 1..16: mol_type = other DNA
atgcaaagcgcggtgg

SEQ ID NO: 124 - DNA, 15 bp, synthetic construct
  misc_feature 1..15: note = antisense oligonucleotides hybridizing with murine ANGPLT4
  source 1..15: mol_type = other DNA
atgcaaagcgcggtg

SEQ ID NO: 125 - DNA, 15 bp, synthetic construct
  misc_feature 1..15: note = antisense oligonucleotides hybridizing with murine ANGPLT4
  source 1..15: mol_type = other DNA
cgctccacgtgttcg

SEQ ID NO: 126 - DNA, 16 bp, synthetic construct
  misc_feature 1..16: note = antisense oligonucleotides hybridizing with murine ANGPLT4
  source 1..16: mol_type = other DNA
gcgctccacgtgttcg

SEQ ID NO: 127 - DNA, 17 bp, synthetic construct
  misc_feature 1..17: note = antisense oligonucleotides hybridizing with murine ANGPLT4
  source 1..17: mol_type = other DNA
tgagtccgccattaagg

SEQ ID NO: 128 - DNA, 16 bp, synthetic construct
  misc_feature 1..16: note = antisense oligonucleotides hybridizing with murine ANGPLT4
  source 1..16: mol_type = other DNA
atgactgagtccgcca

SEQ ID NO: 129 - DNA, 16 bp, synthetic construct
  misc_feature 1..16: note = antisense oligonucleotides hybridizing with murine ANGPLT4
  source 1..16: mol_type = other DNA
tatgactgagtccgcc

SEQ ID NO: 130 - DNA, 17 bp, synthetic construct
  misc_feature 1..17: note = antisense oligonucleotides hybridizing with murine ANGPLT4
  source 1..17: mol_type = other DNA
aatatgactgagtccgc

SEQ ID NO: 131 - DNA, 16 bp, synthetic construct
  misc_feature 1..16: note = antisense oligonucleotides hybridizing with murine ANGPLT4
  source 1..16: mol_type = other DNA
aatatgactgagtccg

SEQ ID NO: 132 - DNA, 17 bp, synthetic construct
  misc_feature 1..17: note = antisense oligonucleotides hybridizing with murine ANGPLT4
  source 1..17: mol_type = other DNA
cctagtagatgcgccta

SEQ ID NO: 133 - DNA, 17 bp, synthetic construct
  misc_feature 1..17: note = antisense oligonucleotides hybridizing with murine ANGPLT4
  source 1..17: mol_type = other DNA
tcgttagtctaagtaga

SEQ ID NO: 134 - DNA, 17 bp, synthetic construct
  misc_feature 1..17: note = antisense oligonucleotides hybridizing with murine ANGPLT4
  source 1..17: mol_type = other DNA
agtactagactcgttag

SEQ ID NO: 135 - DNA, 17 bp, synthetic construct
  misc_feature 1..17: note = antisense oligonucleotides hybridizing with murine ANGPLT4
  source 1..17: mol_type = other DNA
tcggcaacctcctctta

SEQ ID NO: 136 - DNA, 17 bp, synthetic construct
  misc_feature 1..17: note = antisense oligonucleotides hybridizing with murine ANGPLT4
  source 1..17: mol_type = other DNA
gctttatgtcggcaacc

SEQ ID NO: 137 - DNA, 17 bp, synthetic construct
  misc_feature 1..17: note = antisense oligonucleotides hybridizing with murine ANGPLT4
  source 1..17: mol_type = other DNA
ttgactcatgccataac

SEQ ID NO: 138 - DNA, 17 bp, synthetic construct
  misc_feature 1..17: note = antisense oligonucleotides hybridizing with murine ANGPLT4
  source 1..17: mol_type = other DNA
gttaacggctaataaga

SEQ ID NO: 139 - DNA, 17 bp, synthetic construct
  misc_feature 1..17: note = antisense oligonucleotides hybridizing with murine ANGPLT4
  source 1..17: mol_type = other DNA
tctggttaacggctaat

SEQ ID NO: 140 - DNA, 17 bp, synthetic construct
  misc_feature 1..17: note = antisense oligonucleotides hybridizing with murine ANGPLT4
  source 1..17: mol_type = other DNA
actttagctccttatga

SEQ ID NO: 141 - DNA, 17 bp, synthetic construct
  misc_feature 1..17: note = antisense oligonucleotides hybridizing with murine ANGPLT4
  source 1..17: mol_type = other DNA
gatcgaaactgttatgt

SEQ ID NO: 142 - DNA, 17 bp, synthetic construct
  misc_feature 1..17: note = antisense oligonucleotides hybridizing with murine ANGPLT4
  source 1..17: mol_type = other DNA
tcctctcatccaatcgg

SEQ ID NO: 143 - DNA, 17 bp, synthetic construct
  misc_feature 1..17: note = antisense oligonucleotides hybridizing with murine ANGPLT4
  source 1..17: mol_type = other DNA
acggctatgtctgttac

SEQ ID NO: 144 - DNA, 17 bp, synthetic construct
  misc_feature 1..17: note = antisense oligonucleotides hybridizing with murine ANGPLT4
  source 1..17: mol_type = other DNA
ggacctgtaaccaccta

SEQ ID NO: 145 - DNA, 17 bp, synthetic construct
  misc_feature 1..17: note = antisense oligonucleotides hybridizing with murine ANGPLT4
  source 1..17: mol_type = other DNA
gtattgtcgctgatgaa

SEQ ID NO: 146 - DNA, 17 bp, synthetic construct
  misc_feature 1..17: note = antisense oligonucleotides hybridizing with murine ANGPLT4
  source 1..17: mol_type = other DNA
gatttgcctaaactcgt

SEQ ID NO: 147 - DNA, 17 bp, synthetic construct
  misc_feature 1..17: note = antisense oligonucleotides hybridizing with murine ANGPLT4
  source 1..17: mol_type = other DNA
gagcttgcgatgcctgt

SEQ ID NO: 148 - DNA, 17 bp, synthetic construct
  misc_feature 1..17: note = antisense oligonucleotides hybridizing with murine ANGPLT4
  source 1..17: mol_type = other DNA
gtagatgactaggcctg

SEQ ID NO: 149 - DNA, 17 bp, synthetic construct
  misc_feature 1..17: note = antisense oligonucleotides hybridizing with murine ANGPLT4
  source 1..17: mol_type = other DNA
tcaatggaagcgcttta

SEQ ID NO: 150 - DNA, 17 bp, synthetic construct
  misc_feature 1..17: note = antisense oligonucleotides hybridizing with murine ANGPLT4
  source 1..17: mol_type = other DNA
gtagcagacttgcacta

SEQ ID NO: 151 - DNA, 17 bp, synthetic construct
  misc_feature 1..17: note = antisense oligonucleotides hybridizing with murine ANGPLT4
  source 1..17: mol_type = other DNA
ctcatgttaggtaggtt

SEQ ID NO: 152 - DNA, 17 bp, synthetic construct
  misc_feature 1..17: note = antisense oligonucleotides hybridizing with murine ANGPLT4
  source 1..17: mol_type = other DNA
acgtggccaaagacaat

SEQ ID NO: 153 - DNA, 24 bp, synthetic construct
  misc_feature 1..24: note = positive control CpG oligonucleotide
  source 1..24: mol_type = other DNA
tcgtcgttttgtcgttttgtcgtt

SEQ ID NO: 154 - DNA, 16 bp, synthetic construct
  misc_feature 1..16: note = antisense oligonucleotide
  source 1..16: mol_type = other DNA
caccggagagcatcga

SEQ ID NO: 155 - DNA, 17 bp, synthetic construct
  misc_feature 1..17: note = antisense oligonucleotide
  source 1..17: mol_type = other DNA
cacggcatgtaaggaag

SEQ ID NO: 156 - DNA, 17 bp, synthetic construct
  misc_feature 1..17: note = antisense oligonucleotide
  source 1..17: mol_type = other DNA
acacacggcatgtaagg

SEQ ID NO: 157 - DNA, 17 bp, synthetic construct
  misc_feature 1..17: note = antisense oligonucleotide
  source 1..17: mol_type = other DNA
tagtctcgacagcaggt

SEQ ID NO: 158 - DNA, 17 bp, synthetic construct
  misc_feature 1..17: note = antisense oligonucleotide
  source 1..17: mol_type = other DNA
ggttcgagatgaacgga

SEQ ID NO: 159 - DNA, 16 bp, synthetic construct
  misc_feature 1..16: note = antisense oligonucleotide
  source 1..16: mol_type = other DNA
tcgagatgaacggaga

SEQ ID NO: 160 - DNA, 17 bp, synthetic construct
  misc_feature 1..17: note = antisense oligonucleotide
  source 1..17: mol_type = other DNA
tctgcatcggacacacg

SEQ ID NO: 161 - DNA, 17 bp, synthetic construct
  misc_feature 1..17: note = antisense oligonucleotide
  source 1..17: mol_type = other DNA
aacttagagaaccgcga

SEQ ID NO: 162 - DNA, 17 bp, synthetic construct
  misc_feature 1..17: note = antisense oligonucleotide
  source 1..17: mol_type = other DNA
gccgtgaacttagagaa

SEQ ID NO: 163 - DNA, 16 bp, synthetic construct
  misc_feature 1..16: note = antisense oligonucleotide
  source 1..16: mol_type = other DNA
ttagagaaccgcgagt

SEQ ID NO: 164 - DNA, 17 bp, synthetic construct
  misc_feature 1..17: note = antisense oligonucleotide
  source 1..17: mol_type = other DNA
tgaccaggaagacgctt

SEQ ID NO: 165 - DNA, 17 bp, synthetic construct
  misc_feature 1..17: note = antisense oligonucleotide
  source 1..17: mol_type = other DNA
tcgaaatgagtctgcac

SEQ ID NO: 166 - DNA, 17 bp, synthetic construct
  misc_feature 1..17: note = antisense oligonucleotide
  source 1..17: mol_type = other DNA
cgttgagcacgtctgga

SEQ ID NO: 167 - DNA, 16 bp, synthetic construct
  misc_feature 1..16: note = antisense oligonucleotide
  source 1..16: mol_type = other DNA
cgttgagcacgtctgg

SEQ ID NO: 168 - DNA, 17 bp, synthetic construct
  misc_feature 1..17: note = antisense oligonucleotide
  source 1..17: mol_type = other DNA
cacacgtccagttctca

SEQ ID NO: 169 - DNA, 17 bp, synthetic construct
  misc_feature 1..17: note = antisense oligonucleotide
  source 1..17: mol_type = other DNA
gcggatcggtcaggagt

SEQ ID NO: 170 - DNA, 17 bp, synthetic construct
  misc_feature 1..17: note = antisense oligonucleotide
  source 1..17: mol_type = other DNA
taccacacaccgtcctg

SEQ ID NO: 171 - DNA, 17 bp, synthetic construct
  misc_feature 1..17: note = antisense oligonucleotide
  source 1..17: mol_type = other DNA
ccttgtaccacacaccg

SEQ ID NO: 172 - DNA, 16 bp, synthetic construct
  misc_feature 1..16: note = antisense oligonucleotide
  source 1..16: mol_type = other DNA
ttgtaccacacaccgt

SEQ ID NO: 173 - DNA, 17 bp, synthetic construct
  misc_feature 1..17: note = antisense oligonucleotide
  source 1..17: mol_type = other DNA
cttaacagtggatgacc

SEQ ID NO: 174 - DNA, 16 bp, synthetic construct
  misc_feature 1..16: note = antisense oligonucleotide
  source 1..16: mol_type = other DNA
cgaggacggtttttat

SEQ ID NO: 175 - DNA, 17 bp, synthetic construct
  misc_feature 1..17: note = antisense oligonucleotide
  source 1..17: mol_type = other DNA
aggatccgctcagctcg

SEQ ID NO: 176 - DNA, 17 bp, synthetic construct
  misc_feature 1..17: note = antisense oligonucleotide
  source 1..17: mol_type = other DNA
tcgtgtgaggatccgct

SEQ ID NO: 177 - DNA, 17 bp, synthetic construct
  misc_feature 1..17: note = antisense oligonucleotide
  source 1..17: mol_type = other DNA
aatcggatcacagtcgt

SEQ ID NO: 178 - DNA, 17 bp, synthetic construct
  misc_feature 1..17: note = antisense oligonucleotide
  source 1..17: mol_type = other DNA
tagcacggcggtggcgg

SEQ ID NO: 179 - DNA, 16 bp, synthetic construct
  misc_feature 1..16: note = antisense oligonucleotide
  source 1..16: mol_type = other DNA
tagcacggcggtggcg

SEQ ID NO: 180 - DNA, 16 bp, synthetic construct
  misc_feature 1..16: note = antisense oligonucleotide
  source 1..16: mol_type = other DNA
gcggcgacttggactg

SEQ ID NO: 181 - DNA, 16 bp, synthetic construct
  misc_feature 1..16: note = antisense oligonucleotide
  source 1..16: mol_type = other DNA
ggacgcaaagcgcggc

SEQ ID NO: 182 - DNA, 16 bp, synthetic construct
  misc_feature 1..16: note = antisense oligonucleotide
  source 1..16: mol_type = other DNA
aggacgcaaagcgcgg

SEQ ID NO: 183 - DNA, 17 bp, synthetic construct
  misc_feature 1..17: note = antisense oligonucleotide
  source 1..17: mol_type = other DNA
gcggctgacattgtgag

SEQ ID NO: 184 - DNA, 17 bp, synthetic construct
  misc_feature 1..17: note = antisense oligonucleotide
  source 1..17: mol_type = other DNA
ataggccgtgtcctcgc

SEQ ID NO: 185 - DNA, 17 bp, synthetic construct
  misc_feature 1..17: note = antisense oligonucleotide
  source 1..17: mol_type = other DNA
tataggccgtgtcctcg

SEQ ID NO: 186 - DNA, 17 bp, synthetic construct
  misc_feature 1..17: note = antisense oligonucleotide
  source 1..17: mol_type = other DNA
agtactggccgttgagg

SEQ ID NO: 187 - DNA, 17 bp, synthetic construct
  misc_feature 1..17: note = antisense oligonucleotide
  source 1..17: mol_type = other DNA
gaagtactggccgttga

SEQ ID NO: 188 - DNA, 17 bp, synthetic construct
  misc_feature 1..17: note = antisense oligonucleotide
  source 1..17: mol_type = other DNA
tcttaagcttctgccgc

SEQ ID NO: 189 - DNA, 17 bp, synthetic construct
  misc_feature 1..17: note = antisense oligonucleotide
  source 1..17: mol_type = other DNA
agtcaccgtctttcgtg

SEQ ID NO: 190 - DNA, 17 bp, synthetic construct
  misc_feature 1..17: note = antisense oligonucleotide
  source 1..17: mol_type = other DNA
cgccattgaggccagtc

SEQ ID NO: 191 - DNA, 17 bp, synthetic construct
  misc_feature 1..17: note = antisense oligonucleotide
  source 1..17: mol_type = other DNA
ctgagtccgccattgag

SEQ ID NO: 192 - DNA, 17 bp, synthetic construct
  misc_feature 1..17: note = antisense oligonucleotide
  source 1..17: mol_type = other DNA
attggcgcctgcttgtg

SEQ ID NO: 193 - DNA, 17 bp, synthetic construct
  misc_feature 1..17: note = antisense oligonucleotide
  source 1..17: mol_type = other DNA
ataccattggcgcctgc

SEQ ID NO: 194 - DNA, 17 bp, synthetic construct
  misc_feature 1..17: note = antisense oligonucleotide
  source 1..17: mol_type = other DNA
tgagtccgccattgagg

SEQ ID NO: 195 - DNA, 17 bp, synthetic construct
  misc_feature 1..17: note = antisense oligonucleotide
  source 1..17: mol_type = other DNA
actgagtccgccattga

SEQ ID NO: 196 - DNA, 17 bp, synthetic construct
  misc_feature 1..17: note = antisense oligonucleotide
  source 1..17: mol_type = other DNA
tgactgagtccgccatt

SEQ ID NO: 197 - DNA, 17 bp, synthetic construct
  misc_feature 1..17: note = antisense oligonucleotide
  source 1..17: mol_type = other DNA
tgtgactgagtccgcca

SEQ ID NO: 198 - DNA, 17 bp, synthetic construct
  misc_feature 1..17: note = antisense oligonucleotide
  source 1..17: mol_type = other DNA
ccccgtcagtcaatgtg

SEQ ID NO: 199 - DNA, 17 bp, synthetic construct
  misc_feature 1..17: note = antisense oligonucleotide
  source 1..17: mol_type = other DNA
ggtccccgtcagtcaat

SEQ ID NO: 200 - DNA, 17 bp, synthetic construct
  misc_feature 1..17: note = antisense oligonucleotide
  source 1..17: mol_type = other DNA
ttcacggttgcacctaa

SEQ ID NO: 201 - DNA, 16 bp, synthetic construct
  misc_feature 1..16: note = antisense oligonucleotide
  source 1..16: mol_type = other DNA
ataagcgtttcacggt

SEQ ID NO: 202 - DNA, 17 bp, synthetic construct
  misc_feature 1..17: note = antisense oligonucleotide
  source 1..17: mol_type = other DNA
gctaggactccggaacg

SEQ ID NO: 203 - DNA, 17 bp, synthetic construct
  misc_feature 1..17: note = antisense oligonucleotide
  source 1..17: mol_type = other DNA
cagcaacgctaggactc

SEQ ID NO: 204 - DNA, 17 bp, synthetic construct
  misc_feature 1..17: note = antisense oligonucleotide
  source 1..17: mol_type = other DNA
gtgcagcaacgctagga

SEQ ID NO: 205 - DNA, 17 bp, synthetic construct
  misc_feature 1..17: note = antisense oligonucleotide
  source 1..17: mol_type = other DNA
ccgcgcatagcaccagg

SEQ ID NO: 206 - DNA, 17 bp, synthetic construct
  misc_feature 1..17: note = antisense oligonucleotide
  source 1..17: mol_type = other DNA
atgcaaagcgcggtggc

SEQ ID NO: 207 - DNA, 17 bp, synthetic construct
  misc_feature 1..17: note = antisense oligonucleotide
  source 1..17: mol_type = other DNA
ccacgtgttcgcgcagc

SEQ ID NO: 208 - DNA, 17 bp, synthetic construct
  misc_feature 1..17: note = antisense oligonucleotide
  source 1..17: mol_type = other DNA
agcgttaccacaggcag

SEQ ID NO: 209 - DNA, 17 bp, synthetic construct
  misc_feature 1..17: note = antisense oligonucleotide
  source 1..17: mol_type = other DNA
tctatcctcggagtctt

SEQ ID NO: 210 - DNA, 17 bp, synthetic construct
  misc_feature 1..17: note = antisense oligonucleotide
  source 1..17: mol_type = other DNA
ctcgaagtcttgtctac

SEQ ID NO: 211 - DNA, 17 bp, synthetic construct
  misc_feature 1..17: note = antisense oligonucleotide
  source 1..17: mol_type = other DNA
gaagtattgtccattga

SEQ ID NO: 212 - DNA, 17 bp, synthetic construct
  misc_feature 1..17: note = antisense oligonucleotide
  source 1..17: mol_type = other DNA
cagaggatagtagcggc

SEQ ID NO: 213 - DNA, 17 bp, synthetic construct
  misc_feature 1..17: note = antisense oligonucleotide
  source 1..17: mol_type = other DNA
cagtctctccagttacg

SEQ ID NO: 214 - DNA, 16 bp, synthetic construct
  misc_feature 1..16: note = antisense oligonucleotide
  source 1..16: mol_type = other DNA
gcgagaagtgatattc

SEQ ID NO: 215 - DNA, 17 bp, synthetic construct
  misc_feature 1..17: note = antisense oligonucleotide
  source 1..17: mol_type = other DNA
tcaatatgactgagtcc

SEQ ID NO: 216 - DNA, 17 bp, synthetic construct
  misc_feature 1..17: note = antisense oligonucleotide
  source 1..17: mol_type = other DNA
tccagtcagtcaatatg

SEQ ID NO: 217 - DNA, 17 bp, synthetic construct
  misc_feature 1..17: note = antisense oligonucleotide
  source 1..17: mol_type = other DNA
ctccagtcagtcaatat

SEQ ID NO: 218 - DNA, 17 bp, synthetic construct
  misc_feature 1..17: note = antisense oligonucleotide
  source 1..17: mol_type = other DNA
gagtcctagtagatgcg

SEQ ID NO: 219 - DNA, 17 bp, synthetic construct
  misc_feature 1..17: note = antisense oligonucleotide
  source 1..17: mol_type = other DNA
gttagtctaagtagagt

SEQ ID NO: 220 - DNA, 16 bp, synthetic construct
  misc_feature 1..16: note = antisense oligonucleotide
  source 1..16: mol_type = other DNA
agtactagactcgtta

SEQ ID NO: 221 - DNA, 17 bp, synthetic construct
  misc_feature 1..17: note = antisense oligonucleotide
  source 1..17: mol_type = other DNA
gcaacctcctcttattc

SEQ ID NO: 222 - DNA, 17 bp, synthetic construct
  misc_feature 1..17: note = antisense oligonucleotide
  source 1..17: mol_type = other DNA
aagatatgcaaggctag

SEQ ID NO: 223 - DNA, 17 bp, synthetic construct
  misc_feature 1..17: note = antisense oligonucleotide
  source 1..17: mol_type = other DNA
gactcatgccataacaa

SEQ ID NO: 224 - DNA, 17 bp, synthetic construct
  misc_feature 1..17: note = antisense oligonucleotide
  source 1..17: mol_type = other DNA
gtggacctgacaagaag

SEQ ID NO: 225 - DNA, 17 bp, synthetic construct
  misc_feature 1..17: note = antisense oligonucleotide
  source 1..17: mol_type = other DNA
aacggctaataagattt

SEQ ID NO: 226 - DNA, 17 bp, synthetic construct
  misc_feature 1..17: note = antisense oligonucleotide
  source 1..17: mol_type = other DNA
gttctggttaacggcta

SEQ ID NO: 227 - DNA, 17 bp, synthetic construct
  misc_feature 1..17: note = antisense oligonucleotide
  source 1..17: mol_type = other DNA
ggtgtgcttactctggt

SEQ ID NO: 228 - DNA, 17 bp, synthetic construct
  misc_feature 1..17: note = antisense oligonucleotide
  source 1..17: mol_type = other DNA
cctagaaattgtgatcg

SEQ ID NO: 229 - DNA, 17 bp, synthetic construct
  misc_feature 1..17: note = antisense oligonucleotide
  source 1..17: mol_type = other DNA
aacgaataggcatgaac

SEQ ID NO: 230 - DNA, 17 bp, synthetic construct
  misc_feature 1..17: note = antisense oligonucleotide
  source 1..17: mol_type = other DNA
actttcacctagttggc

SEQ ID NO: 231 - DNA, 17 bp, synthetic construct
  misc_feature 1..17: note = antisense oligonucleotide
  source 1..17: mol_type = other DNA
ctctacttggctaggct

SEQ ID NO: 232 - DNA, 17 bp, synthetic construct
  misc_feature 1..17: note = antisense oligonucleotide
  source 1..17: mol_type = other DNA
ggtactctgaattagta

SEQ ID NO: 233 - DNA, 17 bp, synthetic construct
  misc_feature 1..17: note = antisense oligonucleotide
  source 1..17: mol_type = other DNA
tgtaaccacctaaagcc

SEQ ID NO: 234 - DNA, 17 bp, synthetic construct
  misc_feature 1..17: note = antisense oligonucleotide
  source 1..17: mol_type = other DNA
aggtattgtcgctgatg

SEQ ID NO: 235 - DNA, 17 bp, synthetic construct
  misc_feature 1..17: note = antisense oligonucleotide
  source 1..17: mol_type = other DNA
gaacagaggtattgtcg

SEQ ID NO: 236 - DNA, 17 bp, synthetic construct
  misc_feature 1..17: note = antisense oligonucleotide
  source 1..17: mol_type = other DNA
catgtatcacaccttcc

SEQ ID NO: 237 - DNA, 17 bp, synthetic construct
  misc_feature 1..17: note = antisense oligonucleotide
  source 1..17: mol_type = other DNA
taaactcgttcctgcct

SEQ ID NO: 238 - DNA, 17 bp, synthetic construct
  misc_feature 1..17: note = antisense oligonucleotide
  source 1..17: mol_type = other DNA
gcctaaactcgttcctg

SEQ ID NO: 239 - DNA, 17 bp, synthetic construct
  misc_feature 1..17: note = antisense oligonucleotide
  source 1..17: mol_type = other DNA
atctcaggagcttatac

SEQ ID NO: 240 - DNA, 17 bp, synthetic construct
  misc_feature 1..17: note = antisense oligonucleotide
  source 1..17: mol_type = other DNA
acaagctgcataatagg

SEQ ID NO: 241 - DNA, 17 bp, synthetic construct
  misc_feature 1..17: note = antisense oligonucleotide
  source 1..17: mol_type = other DNA
tatcactgagcttgcga

SEQ ID NO: 242 - DNA, 17 bp, synthetic construct
  misc_feature 1..17: note = antisense oligonucleotide
  source 1..17: mol_type = other DNA
aatggaagcgctttacc

SEQ ID NO: 243 - DNA, 17 bp, synthetic construct
  misc_feature 1..17: note = antisense oligonucleotide
  source 1..17: mol_type = other DNA
tagcagacttgcactat

SEQ ID NO: 244 - DNA, 17 bp, synthetic construct
  misc_feature 1..17: note = antisense oligonucleotide
  source 1..17: mol_type = other DNA
aggctcaactctcgcac

SEQ ID NO: 245 - DNA, 17 bp, synthetic construct
  misc_feature 1..17: note = antisense oligonucleotide
  source 1..17: mol_type = other DNA
aagagctagtactgtag

SEQ ID NO: 246 - DNA, 17 bp, synthetic construct
  misc_feature 1..17: note = antisense oligonucleotide
  source 1..17: mol_type = other DNA
tataatttgatcctgac

SEQ ID NO: 247 - DNA, 17 bp, synthetic construct
  misc_feature 1..17: note = antisense oligonucleotide
  source 1..17: mol_type = other DNA
ggttctctgccaaatga

SEQ ID NO: 248 - DNA, 17 bp, synthetic construct
  misc_feature 1..17: note = antisense oligonucleotide
  source 1..17: mol_type = other DNA
cgtggccaaagacaatt

SEQ ID NO: 249 - DNA, 16 bp, synthetic construct
  misc_feature 1..16: note = Negative control oligonucleotide
  source 1..16: mol_type = other DNA
tacgcgcggttgttta

SEQ ID NO: 250 - DNA, 16 bp, synthetic construct
  misc_feature 1..16: note = Negative control oligonucleotide
  source 1..16: mol_type = other DNA
ttagcgcgcgaatatg

SEQ ID NO: 251 - DNA, 17 bp, synthetic construct
  misc_feature 1..17: note = Negative control oligonucleotide
  source 1..17: mol_type = other DNA
cgaataaccgtcgtgtt

SEQ ID NO: 252 - DNA, 17 bp, synthetic construct
  misc_feature 1..17: note = Negative control oligonucleotide
  source 1..17: mol_type = other DNA
gactcgttaaaccgata